(19) United States
(12) Patent Application Publication
(10) Pub. No.: US 2026/0221264 A1
(43) Pub. Date: Jul. 30, 2026

(54) INTERACTIVE COMMUNICATION MANAGEMENT AND INFORMATION DELIVERY SYSTEMS AND METHODS

(71) Applicant: OpticSurg Inc., Wilmington, DE (US)

(72) Inventors: Tran T. Huynh, New York, NY (US); Carl Tönsgård, Heberg (SE); Kyle P. Chadwick, Northridge, CA (US); Michael Rizkalla, Staten Island, NY (US)

(73) Assignee: OpticSurg Inc., Wilmington, DE (US)

(21) Appl. No.: 17/473,825

(22) Filed: Sep. 13, 2021

(51) Int. Cl. G16H 30/40; G06F 3/14; G06T 11/00; G06V 20/20; G16H 10/60; G16H 30/20; G16Y 10/60; H04N 5/262; H04N 5/272

(52) U.S. Cl. CPC G16H 30/40; G06F 3/14; G06T 11/00; G06V 20/20; G16H 10/60; G16H 30/20; H04N 5/2628; H04N 5/272; G06T 2200/24; G06T 2210/41; G06V 2201/03; G16Y 10/60

(57) ABSTRACT

A communication management and information delivery system includes a wearable system configured to display virtual content in an AR environment, which includes a sensor assembly having a sensor configured to capture medical multimedia data in connection with a medical session and a display device to display virtual content in an AR environment. One or more processors are configured to interact with non-transitory memory to perform operations including: facilitating a secure communication between a remote computing device and the wearable system, wherein the remote computing device is configured to display the captured medical multimedia data on a display; generating feedback data associated with at least one of the captured medical multimedia data and electronic content displayed on the display, the feedback data generated based on feedback captured using one or more user input devices associated with the remote computing device; and, processing the feedback data with an annotation application.

### BACKGROUND

Medical procedures (such as surgery, pathology, dissection, autopsy, diagnosis, etc.) healthcare delivery (bedside care, elderly care, etc.), clinical care and collaboration, medical education, etc. (hereinafter sometimes collectively called “medical sessions” or the like), can be complex and multi-pronged processes. In order to successfully perform a medical session, a medical professional (such as a surgeon, nurse, caretaker, etc.), may need to access supplemental information, such as a patient's medical records, medical imaging (such as a Magnetic Resonance Imaging (MRI) scan), endoscopy medical literature, an expert's opinion, etc.

Accessing that supplemental information during a medical session is complicated. For instance, medical images are typically accessed on a computer using an input device, such as a mouse or keyboard. However, it is impossible for the medical professional to use the mouse or keyboard, as they are unsterile and pose a risk of infection. In such situations, the medical professional may request an assistant to operate the computer to retrieve the medical images or other information related to the patient. Such manual coordination of collecting patient data is a time-consuming process. In addition, current medical image viewing equipment utilized in the healthcare facility are cumbersome, ergonomically unfriendly and bulky.

As another example, the medical professional may need to consult with an expert in a remote location during a medical session. The medical professional may request an assistant to call a certain expert, however, such manual coordination is time consuming. Moreover, the remote expert cannot see what the medical professional is seeing unless a video conferencing system is used, which may require additional equipment and manual coordination to capture the medical professional's point of view.

Sharing information for education purposes during a medical session, such as with students, is cumbersome and time consuming.

As another example, the medical professional may need to view a live image of a targeted area of the patient, such as a zoomed-in view of the surgery site or an area of concern. The medical professional may again request an assistant to change an aspect of the view, however, such manual coordination is time-consuming and inefficient.

The success of the medical session may be compromised without timely supplemental information. Moreover, the healthcare facility resources, such as the operating room, medical equipment, medical staff, special surgeons, etc., stay underutilized because of such manual management of the medical sessions.

Accordingly, there is a need for improved communication management and information delivery system and method that can improve the access to supplemental information, the process for accessing the supplemental information, and the ability to collaborate with remote personnel for improving the overall quality and efficiency of medical sessions, or other similar procedures or applications.

### SUMMARY

In some aspects, the techniques described herein relate to a communication management and information delivery system, including: a wearable system configured to display virtual content in an AR environment, the wearable system including: a sensor assembly having at least one sensor configured to capture medical multimedia data in connection with a medical session; and a display device configured to display virtual content in an AR environment; one or more processors configured to interact with non-transitory memory to perform operations including: facilitating a secure communication between a remote computing device and the wearable system, wherein the remote computing device is configured to display the captured medical multimedia data on a display; generating feedback data associated with at least one of the captured medical multimedia data and electronic content displayed on the display of the remote computing device, the feedback data generated based on feedback captured using one or more user input devices associated with the remote computing device; processing the feedback data with an annotation application including at least one of: a freeze frame module configured to add an annotation to a frozen video feed frame displayed on the display of the remote client computing device, the annotation corresponding to a 2-D location of the one or more user input devices; a live annotation module configured to add an annotation to a live video feed frame displayed on the display of the remote client computing device, the annotation corresponding to a 2-D location of the one or more user input devices; an object type annotation module configured to add an annotation corresponding to a type of an object being annotated; and device specific annotation module configured to add an annotation corresponding to processing capabilities of the wearable system; receiving the feedback data from the remote device; and displaying on the display device of the wearable system the feedback data superimposed over a real-world view of at least one of the medical multimedia data, electronic content, and virtual content.

In some aspects, the techniques described herein relate to a communication management and information delivery system, including: a wearable system configured to display virtual content in an AR environment, the wearable system including: a sensor assembly having at least one sensor configured to capture medical multimedia data in connection with a medical session; and a display device configured to display virtual content in an AR environment; a remote computing device in secure communication with the wearable system, the remote computing device configured to display captured medical multimedia data on a display, the remote computing device having one or more user input devices configured to capture feedback data associated with at least one of the medical multimedia data and electronic content; one or more processors configured to interact with non-transitory memory configured to perform operations including at least one of: adding an annotation to at least one of the medical multimedia data and electronic content with an annotation application configured to process the feedback data; and processing one or more depth estimation signals captured by the sensor assembly to map an annotation to a depth of an object viewed by a wearer of the wearable system with a depth estimation application; wherein the display device of the wearable system is configured to display the feedback data superimposed over a real-world view of at least one of the medical multimedia data, electronic content, and virtual content.

In some aspects, the techniques described herein relate to a wearable system configured to display virtual content in an AR environment, the wearable system including: a medical multimedia data assembly having at least one sensor configured to capture medical multimedia data in connection with a medical session; a display device configured to display virtual content in an AR environment; and a depth estimation assembly having at least one sensor configured to output one or more depth estimation signals based on sensed visual input that can be processed by a processor having a depth estimation application configured to produce a depth map having pixel values corresponding to a distance between the wearable system and the nearest solid object at that position.

In some aspects, the techniques described herein relate to a method for facilitating a secure communication between a remote computing device and a wearable system, wherein the wearable system is configured to display virtual content in an AR environment and capture medical multimedia data in connection with a medical session, and wherein the remote computing device is configured to display the captured medical multimedia data on a display, the method including: generating feedback data associated with at least one of the captured medical multimedia data and electronic content displayed on the display of the remote computing device, the feedback data generated based on feedback captured using one or more user input devices associated with the remote computing device; processing the feedback data with an annotation application including at least one of: a freeze frame module configured to add an annotation to a frozen video feed frame displayed on the display of the remote client computing device, the annotation corresponding to a 2-D location of the one or more user input devices; a live annotation module configured to add an annotation to a live video feed frame displayed on the display of the remote client computing device, the annotation corresponding to a 2-D location of the one or more user input devices; an object type annotation module configured to add an annotation corresponding to a type of an object being annotated; and device specific annotation module configured to add an annotation corresponding to processing capabilities of the wearable system; receiving the feedback data from the remote device; and displaying on the display device of the wearable system the feedback data superimposed over a real-world view of at least one of the medical multimedia data, electronic content, and virtual content.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 shows an isometric view of an exemplary communication management and information delivery system (“system”) formed in accordance with the present disclosure.

FIG. 2 shows an isometric view of an exemplary wearable computing device for use with the exemplary system of FIG. 1.

FIGS. 3A and 3B show a block diagram of exemplary system architecture of a smartglasses computing device and smartglasses for use with the systems and methods described herein.

FIG. 4 shows a block diagram of exemplary system architecture of an application computing device for use with the systems and methods described herein.

FIGS. 5A-5K show block diagrams of exemplary applications for use with the application computing device of FIG. 4.

FIG. 6 shows a block diagram of exemplary system architecture of a remote client computing device for use with the systems and methods described herein.

FIG. 7 shows a block diagram of exemplary system architecture of a healthcare facility computing system for use with the systems and methods described herein.

FIG. 8 shows a swim diagram illustrating the various ways components of the system of FIG. 1 can interact.

FIG. 9 shows a flowchart illustrating a method for initiating a communication session for use with the system of FIG. 1.

FIGS. 10-31 show screen shots of exemplary GUI displays of a remote computing device for use with the systems and methods described herein, wherein the GUI displays of FIGS. 13-31 show a live video feed of a wearable computing device as a background.

FIGS. 32-45 show screen shots of exemplary GUI displays of a wearable computing device for use with the systems and methods described herein, wherein the GUI displays of FIGS. 32-45 show a real-world scene of a wearer of a wearable computing device as a background.

FIG. 46 shows an example neural network architecture, according to one aspect of the present disclosure.

FIG. 47 shows a block diagram illustrating an example of a computing device.

FIGS. 48-68 show color screen shots of exemplary GUI displays of a remote computing device for use with the systems and methods described herein, wherein the GUI displays of FIGS. 48-68 show a live video feed of a wearable computing device as a background.

FIGS. 69-86 show color screen shots of exemplary GUI displays of a wearable computing device for use with the systems and methods described herein, wherein the GUI displays of FIGS. 69-86 show a real-world scene of a wearer of a wearable computing device as a background.

The patent or application file contains at least one drawing executed in color. Copies of this patent or patent application publication with color drawing(s) will be provided by the Office upon request and payment of the necessary fee.

### DETAILED DESCRIPTION

The present disclosure provides exemplary embodiments of a communication management and information delivery system and method that that incorporates augmented reality (AR) or mixed reality (MR) technology (hereinafter collectively referred to as “AR technology” or “AR”) to optimize medical sessions or other similar procedures or applications. Potential uses for AR in the medical field include surgery, procedures (diagnostic procedure, therapeutic procedure, surgical procedure, emergency room procedure, interventional procedure, bedside procedure, etc.), healthcare delivery, education, simulation, and clinical care such as telemedicine (hereinafter sometimes collectively called “medical sessions”). The medical session may also include capturing medical images, video, chart information, diagnostic readings, and the like.

Accordingly, although exemplary embodiments of a communication management and information delivery system and method will be hereinafter described with reference to a medical procedure, such as a surgery, it should be appreciated that the systems and methods described herein may also be used for any suitable medical session. Moreover, the systems and methods described herein may be adapted for use with other procedures or applications outside of the medical field.

Technology that offers an AR experience overlays a virtual world on top of a user's existing (“real”) surroundings. AR technology differs from devices for virtual reality (VR) that completely immerse a user within a virtual experience. Augmented reality has potential use in many different medical applications.

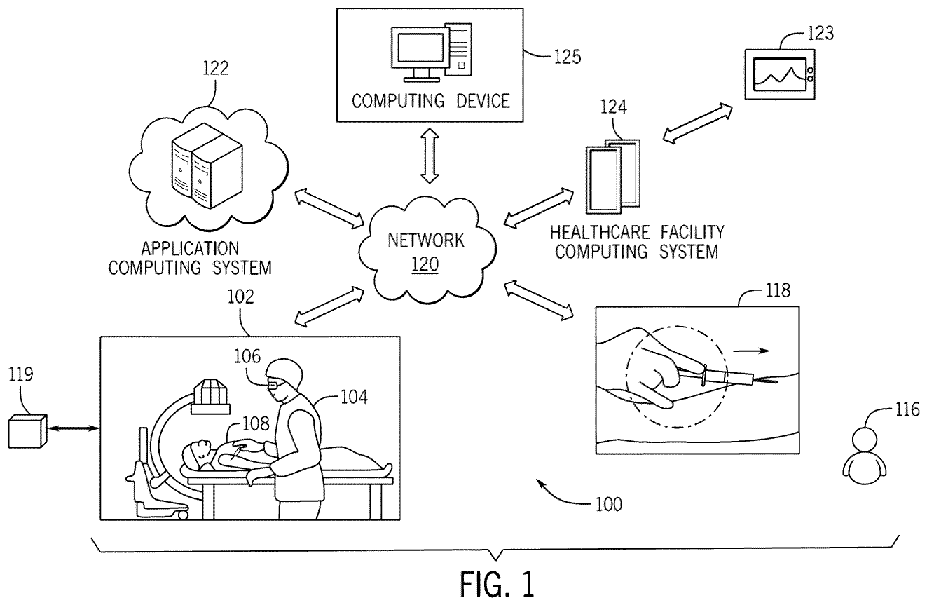

FIG. 1 depicts an exemplary embodiment of a communication management and information delivery system 100 that incorporates AR technology to enable a medical professional 104 located at a healthcare facility 102 to collaborate with at least one remote user 116 associated with a remote client computing device 118 during a medical session. In the exemplary embodiment depicted in FIG. 1, each medical professional 104 dons a wearable AR device 106 (e.g., a pair of smartglasses) while engaging in a medical session with a patient 108. However, it should be appreciated that the medical professional 104 may instead use a handheld device such as a smart phone or tablet.

The medical professional 104 may be any person suited to use the wearable AR device 106 (hereinafter alternatively referred to as “smartglasses 106”), such as a surgeon, a nurse, a caretaker, a professor, a medical assistant, a medical staff representative, an anesthesiologist, an MRI or an X-Ray technician, etc. In that regard, the medical professional 104 may instead be called a smartglasses wearer 104 or an AR user. Moreover, the communication management and information delivery system 100 may support more than one smartglasses wearer or AR user for a given session or interaction with remote user(s).

In general, the communication management and information delivery system 100 is configured such that images captured via the smartglasses 106 may be provided to one or more remote client computing devices 118. Although only one remote client computing device 118 is shown and referenced, it should be appreciated that the system 100 may support a plurality of remote client computing devices 118.

Information from each remote client computing device 118, such as annotations, audio or video instructions, and the like associated with the captured information or data may be transmitted or pushed from the remote client computing device 118 to the smartglasses 106 to instruct or guide the medical practitioner on the care of the patient. The smartglasses 106 include a smartglasses computing device 119 in wired or wireless communication with the smartglasses 106 other otherwise integrated into the smartglasses 106 for carrying out the various required functions. In that regard, it should be appreciated that any reference to a transfer of data to/from the smartglasses 106 may be understood to first be processed by the smartglasses computing device 119.

The communication management and information delivery system 100 includes a communication network 120 that connects the smartglasses 106 to at least one computing system and at least one remote user associated with a remote client computing device. The network 120 may be a centralized network or may include a plurality of sub-networks that may offer a direct or indirect communication between the entities. The network 120 can be a large computer network, such as a local area network (LAN), wide area network (WAN), the Internet, a cellular network, or a combination thereof connecting any number of mobile clients, fixed clients, and servers. In some implementations, each computing system or device can communicate with servers via a virtual private network (VPN), Secure Shell (SSH) tunnel, or other secure network connection. In some implementations, the network 120 can further include a corporate network (e.g., intranet) and one or more wireless access points.

The network 120 may include wired networks, wireless networks and combinations thereof. Some non-limiting examples of the wired networks may include Ethernet, LANs, fiber-optic networks, and the like. Some non-limiting examples of the wireless networks may include cellular networks like GSM/3G/4G/5G/LTE/CDMA networks, wireless LANs, Bluetooth, Wi-Fi or ZigBee networks, and the like. An example of the combination of wired and wireless networks may include the Internet.

In the depicted exemplary embodiment, the system 100 includes an application computing system 122 that may be defined by one or more computing devices (e.g., one or more servers) and one or more computer-readable storage devices (e.g., one or more databases). The application computing system 122 may run one or more platforms, applications, and/or modules configured to support the use of and the communication and interaction between the smartglasses 106 and the remote client computing device 118. The application computing system 122 may be in communication with a secure access console or computing device 125 for allowing an authorized person to interact with the application computing system 122.

The system 100 may further include a healthcare facility computing system 124 that may be defined by one or more computing devices (e.g., one or more servers) and one or more computer-readable storage devices (e.g., one or more databases). The healthcare facility computing system 124 may run one or more platforms, applications, and/or modules configured to facilitate a private network for the healthcare facility 102. Moreover, the healthcare facility computing system 124 may run one or more platforms, applications, and/or modules configured to support a request by a healthcare user (such as the medical professional 104 or other staff) using a computing device (such as the smartglasses 106, medical equipment 123, or a personal computing device such as a cell phone, tablet, laptop, etc.) to communicate with another device in the system 100.

Each of the computing devices and computing systems (e.g., the smartglasses computing device 119, the remote client computing device 118, the application computing system 122, and the healthcare facility computing system 124) can represent various forms of processing devices. Example processing devices can include a desktop computer, a laptop computer, a handheld computer, a tablet computer, a personal digital assistant (PDA), a cellular telephone, a network appliance, a camera, a smart phone, an enhanced general packet radio service (EGPRS) mobile phone, a media player, a navigation device, an email device, a game console, or a combination of any these data processing devices or other data processing devices. The computing devices and computing systems can be provided access to and/or receive application software executed and/or stored on any of the other computing devices and computing systems. The servers of the computing systems 122 and 124 can represent various forms of servers including, but not limited to a web server, an application server, a proxy server, a network server, or a server farm. In some examples, the smartglasses computing device 119 and/or the remote client computing device 118 perform functions of a social network server.

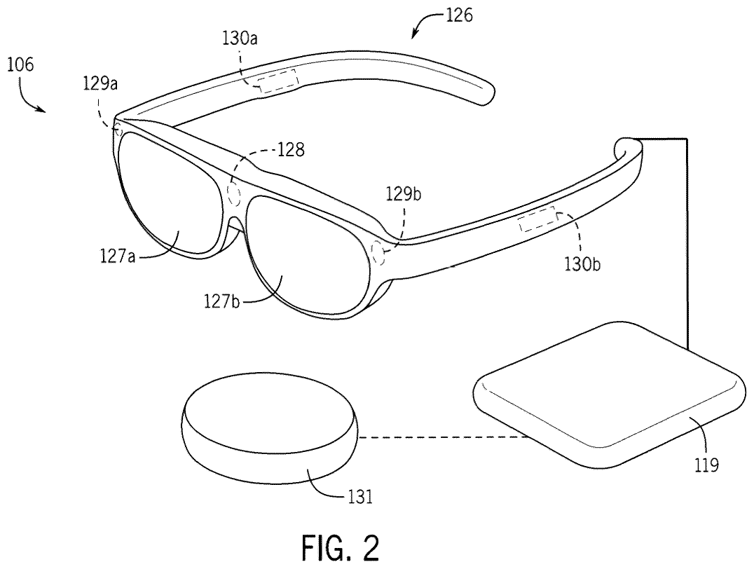

Referring to FIG. 2, an exemplary embodiment of a wearable device will now be described. As noted above, however, the AR user may instead use a handheld device such as a smart phone or tablet. In that regard, it should be appreciated that the term “wearable device” should be broadly interpreted to include any suitable AR device.

Most AR “wearable devices” include an ergonomically assembled head-mounted display that provides a viewable display for human-computer interaction. The wearable device projects a visible image in the user's point of view. It superimposes an image (e.g., a hologram) on the wearer's surrounding real-life environment to create a mixed-reality experience. In the exemplary embodiments described herein, the wearable device is a pair of smartglasses 106. In general, the smartglasses 106 may be any suitable AR wearable computing device capable of supporting at least the AR function of the communication management and information delivery system 100 described herein. In that regard, the smartglasses 106 may be a version of a wearable device now known, such as AR glasses available from Nreal, Microsoft, Google, Occulus, Vuzix, Lenovo, Magic Leap, Apple, etc., or a wearable device later developed. It should be appreciated that the smartglasses 106 shown in FIG. 2 are exemplary only, and any other configuration may instead be used.

In the depicted exemplary embodiment, the smartglasses 106 includes a frame 126 configured to fit a wearer like glasses. Other wearable configurations may instead be used, such a headband, a headset, etc. First and second display screens 127a and 127b are received within openings in the frame 126 through which the wearer may view the AR scene, although it should be appreciated that the first and send display screens 127a and 127b may instead be combined into a single display screen (or simply “display”).

In an example embodiment, the display screens 127a and 127b may be transparent 3-D (three-dimensional) lenses. The lenses may be formed of any material that can suitably display a projected image or graphic. The lenses may facilitate an augmented reality or heads-up display where a projected image or graphic is superimposed over a real-world view as perceived by the wearer through the lenses. In another example embodiment, the images and other information may be projected on the user's retina or displayed on the user's contact lenses.

The smartglasses 106 include a plurality of sensors configured to detect various inputs for supporting the AR experience. For instance, the smartglasses 106 include a plurality of sensors configured to detect various inputs from the real world environment viewed by the wearer (e.g., objects, patient anatomy, medical equipment, environment (such as temperature, sound, light, etc.), etc.), virtual input by the wearer (wearer movements, position tracking, touch free commands via motion, eye gaze, etc.), audible or touch input by the wearer, or other aspects for supporting the AR experience. For instance, the smartglasses 106 may include one or more cameras, proximity sensors, light sensors, inertial measurement units (IMUs), gyroscopes, accelerometers, compasses, and the like to detect the real-world environment and/or wearer movements. Additionally, the smartglasses 106 may include a speaker/microphone, a finger-operated touchpad, etc., to receive audible or touch input by the wearer.

FIG. 2 shows the smartglasses 106 having a first centered forward facing sensor assembly 128, which may include proximity and ambient light sensors and/or a red-green-blue (RGB) camera. The smartglasses 106 are also shown having a first lateral forward facing sensor assembly 129a that may include a first grayscale camera, and a second lateral forward facing sensor assembly 129b that may include a second grayscale camera. The first or second lateral forward facing sensor assembly 129a or 129b may also include an IMU. Further, the forward-facing sensor assemblies 128, 129a or 129b may also include a color camera, spatial computing cameras, LIDAR, radar and/or time of flight cameras, monocular cameras, etc.

The smartglasses 106 are also shown having first and second speaker/microphones 130a and 130b situated on first and second temple portions (not labeled) of the frame 126. Although not shown, the smartglasses 106 may further include an earphone/speaker connector port, an I/O port (such as a USB port) and/or a power charging port. The smartglasses 106 may further include a built-in processing unit and batteries for processing power, or the smartglasses 106 may instead be in wired or wireless connection with the smartglasses computing device 119 optionally controlled through a handheld controller 131.

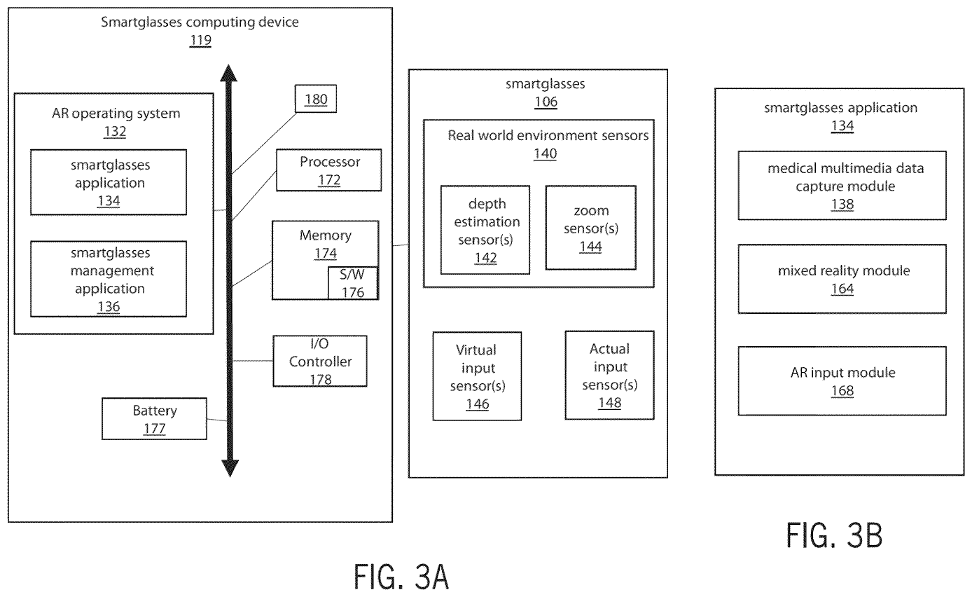

FIG. 3A depicts an exemplary block diagram of the smartglasses computing device 119. The smartglasses computing device 119 is generally configured to provide processing capabilities to the smartglasses 106 and for communicating with the remote client computing device 118 or other computing systems coupled to the network 120 (“network devices”). In other words, the smartglasses computing device 119 is generally configured to provide AR function to the smartglasses 106 and receive, process, and send data between the smartglasses 106 and the network devices. In that regard, any description provided herein of a signal(s) being sent to/from the smartglasses 106 and the network devices may first be processed and repackaged by the smartglasses computing device 119. However, in some embodiments, some or all of the components of the smartglasses computing device 119 are integrated into the smartglasses 106.

The smartglasses computing device 119 may include an AR platform or AR operating system 132 that supports a smartglasses application 134 and a smartglasses management application 136. The smartglasses application 134 is configured to support the AR function of the smartglasses 106 as well as enable the smartglasses 106 to communicate with other network devices, such as the remote client computing device 118.

FIG. 3B depicts an exemplary block diagram of the smartglasses application 134. The smartglasses application 134 may include a medical multimedia data capture module 138 configured to capture medical multimedia data (hereinafter sometimes referred to as data/patient data) associated with the patient 108, medical equipment, etc., during the on-going medical procedure or during medical care of the patient. The captured data is sent to one or more network devices over the network 120, such as the application computing system 122, which may process and send the data to the remote client computing device 118 or other network devices for viewing by the remote user 116.

Referring to both FIGS. 3A and 3B, the medical multimedia data capture module 138 is in communication with the various sensor assemblies of the smartglasses 106, which are configured to capture various types of medical multimedia data. For instance, the medical multimedia data capture module 138 may communicate with one or more real world environment sensors 140 configured to detect various inputs from the real world environment viewed by the wearer, such as objects, patient anatomy, medical equipment, environment (such as temperature, sound, light, etc.). The real-world environment sensors 140 may include one or more of the sensors described above, such one or more cameras, proximity sensors, light sensors, inertial measurement units (IMUs), gyroscopes, accelerometers, compasses, and the like.

In the depicted embodiment, the real world environment sensors 140 may include depth estimation sensors 142 configured to capture depth estimation data used to estimate the depth of objects (e.g., the patient's anatomy, medical equipment, etc.) viewed by the smartglasses wearer 104. The depth estimation sensors 142 may include one or more cameras, such as a grayscale camera(s), a color camera, an RGB camera, spatial computing cameras, LIDAR, radar and/or time of flight cameras, monocular cameras, proximity and ambient light sensors, an IMU, etc. The depth estimation sensors 142 may be integrated into or attached to the smartglasses 106, such as in the first centered forward-facing sensor assembly 128 and/or the first and/or second lateral forward facing sensor assembly 129a and 129b.

The depth estimation sensors 142 may output one or more signals, which may be processed by the medical multimedia data capture module 138 and sent to one or more network devices over the network 120. For instance, the medical multimedia data capture module 138 may send the processed depth estimation sensor signals to the application computing system 122, which may process and the depth estimation data to facilitate a real time visual interaction between the smartglasses wearer 104 and the remote user 116.

The real world environment sensors 140 may further include one or more zoom sensors 144 configured to capture visual zoom data used to produce a zoomed-in image data file for viewing on a display of a computing device, such as the display screen of the smartglasses 106 or the remote client computing device 118. The zoom sensors 144 may include one or more optical sensors, such as cameras, such as a grayscale camera(s), a color camera, an RGB camera, spatial computing cameras, proximity and ambient light sensors, an IMU, etc., which may be the same or different cameras/sensors used as the depth estimation sensors 142. In that regard, the zoom sensors 144 may be integrated into or attached to the smartglasses 106, such as in the first centered forward facing sensor assembly 128 and/or the first and/or second lateral forward facing sensor assembly 129a and 129b.

The zoom sensors 144 may output one or more signals, which may be processed by the smartglasses application 134 for displaying a zoomed-in image on a display screen of the smartglasses 106. In addition or in the alternative, the visual zoom sensor data may be sent to one or more network devices over the network 120, such as the application computing system 122, which may process and send the visual zoom sensor data to the smartglasses 106 or the remote client computing device 118 for viewing by a user or for use by an AI platform for analyzing the captured images.

The medical multimedia data capture module 138 may further communicate with one or more virtual input sensors 142 of the smartglasses 106, which are configured to detect various virtual inputs from the wearer, such as wearer movements, position tracking, touch free commands via motion, eye gaze, etc. The virtual input sensors 142 may include one or more cameras, such as a grayscale camera(s), a color camera, an RGB camera, spatial computing cameras, proximity and ambient light sensors, an IMU, etc., which may be the same or different cameras/sensors used as the depth estimation sensors 142 and/or the zoom sensors 144. In that regard, the virtual input sensors 142 may be integrated into or attached to the smartglasses 106, such as in the first centered forward facing sensor assembly 128 and/or the first and/or second lateral forward facing sensor assembly 129a and 129b.

The virtual input sensors 142 may output one or more signals, which may be processed by the smartglasses application 134 for carrying out one or more commands for interacting with a virtual display. In addition or in the alternative, the virtual input sensor data may be sent to one or more network devices over the network 120, such as the application computing system 122, which may process the virtual input sensor data and carry out one or more commands or functions to support the interactive AR experience.

For instance, the smartglasses wearer 104 can move, pinch and rotate his or her fingers similar to the movement used to operate a smartphone or tablet, and such gestures can be interpreted by the virtual input sensors 142. Further, programmed eye-movements may be used to perform dedicated tasks. For example, the smartglasses 106 may capture an image based on receiving an input command of a wink. As another example, the smartglasses 106 may turn on the display screens 127a and 127b when the wearer's eyes are focused on the display screens for a pre-defined time.

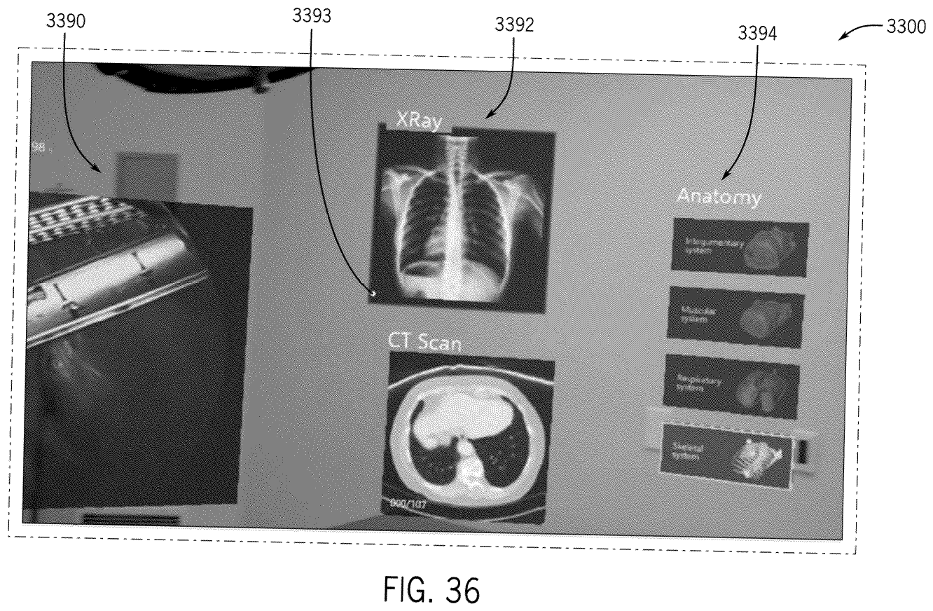

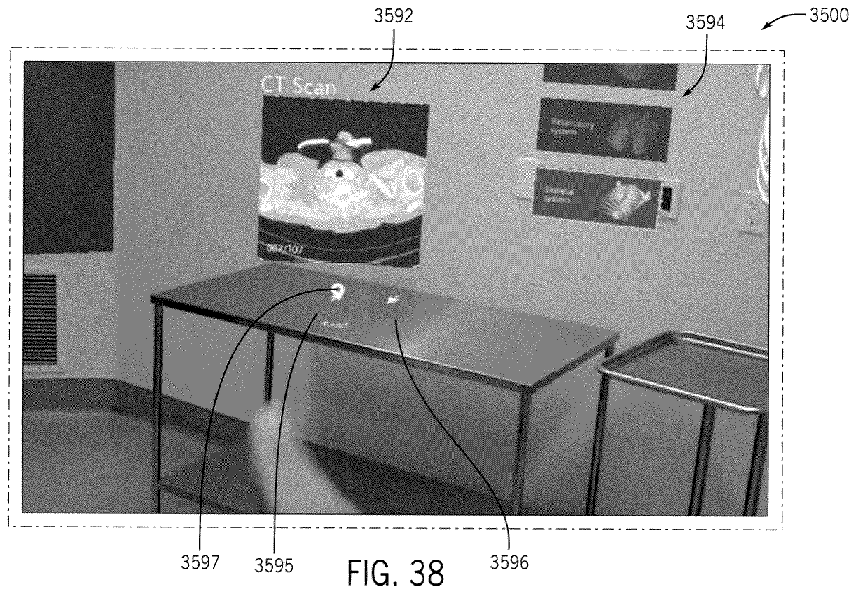

In further examples, the eye gaze of the smartglasses wearer 104 can be used to move a virtual mouse icon for selecting certain virtual objects (see the laser pointer icon 3393 in the screen shot 3300 of FIG. 36 or the toggle icon 3597 in the screen shot 3500 of FIG. 38). Once the virtual icon is aligned with a virtual object, the smartglasses wearer 104 can manipulate the virtual object by hand gestures, voice command, etc. For instance, FIG. 38 illustrates a screen shot 3500 of an exemplary smartglasses GUI display showing manipulation of a CT scan virtual object 3592 through hand gestures.

In further instances, the medical multimedia data capture module 138 may communicate with one or more actual input sensors 148 of the smartglasses 106, which are configured to detect various actual inputs from the wearer, such as audible or touch input by the wearer. The actual input sensors 148 may include one or more speakers/microphones, finger-operated touchpads, etc., to receive audible or touch input or other actual inputs from the wearer. In that regard, the wearer may control certain aspects of the AR experience by voice command.

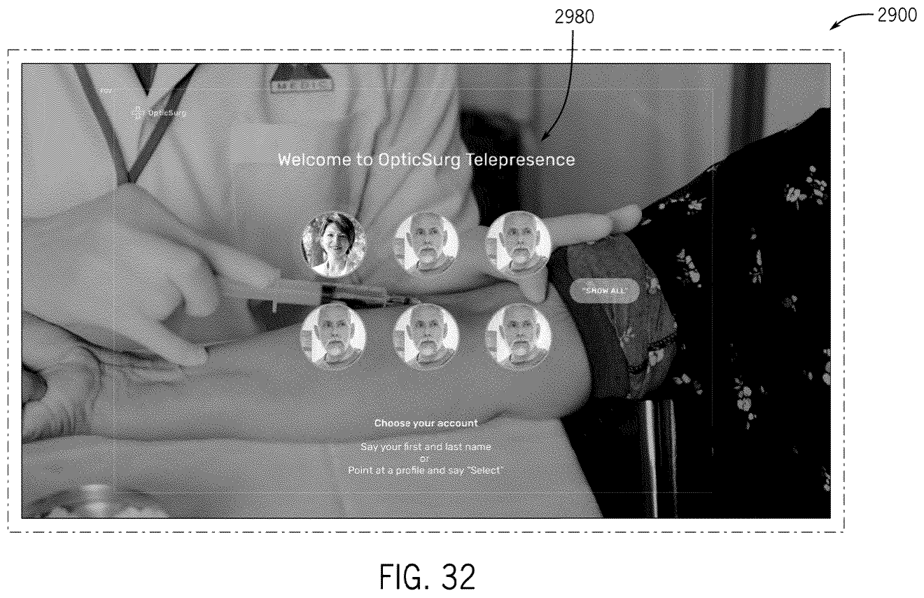

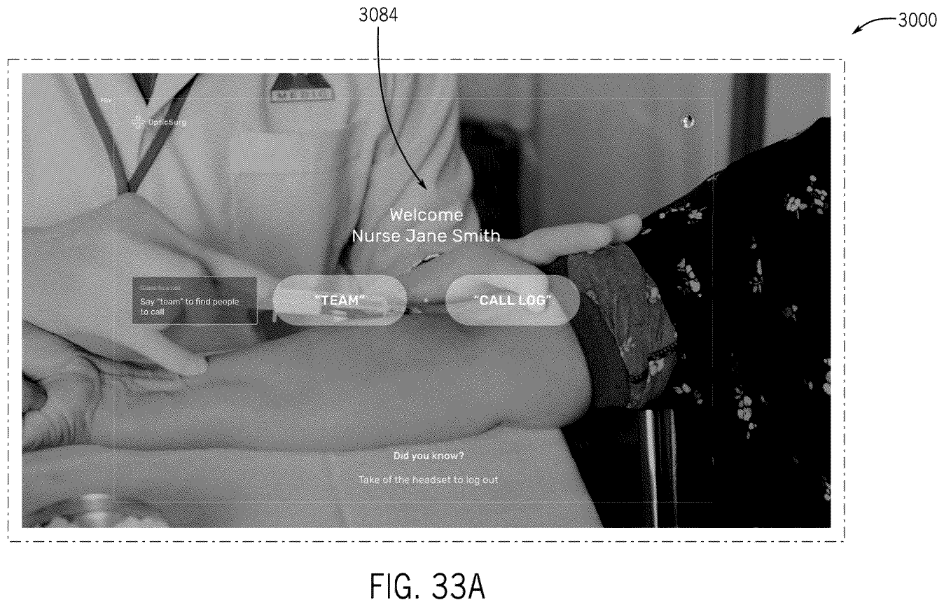

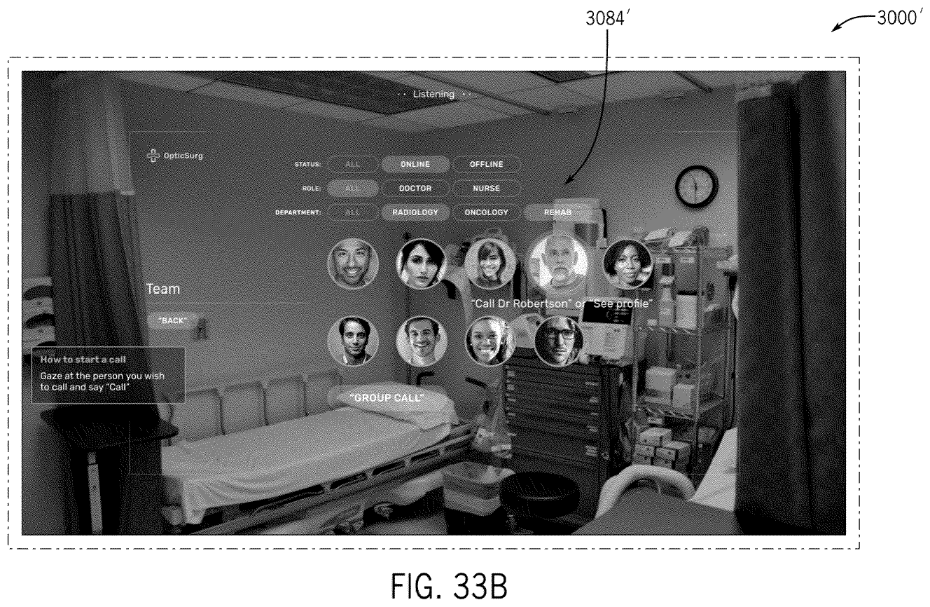

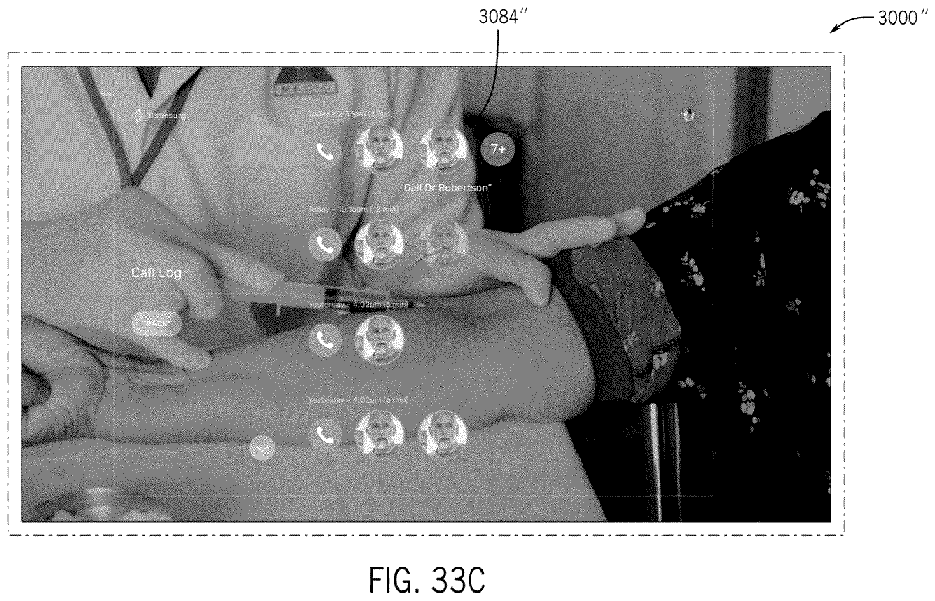

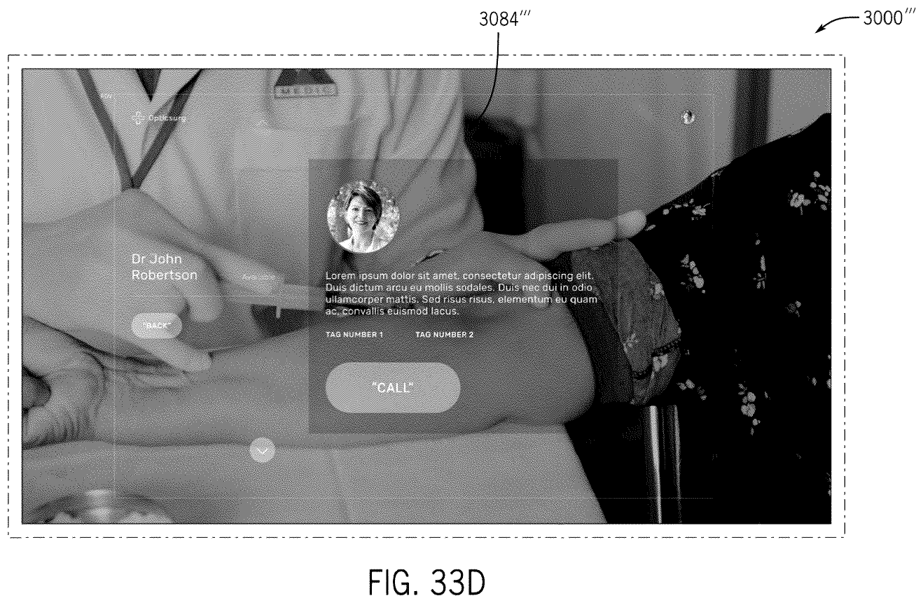

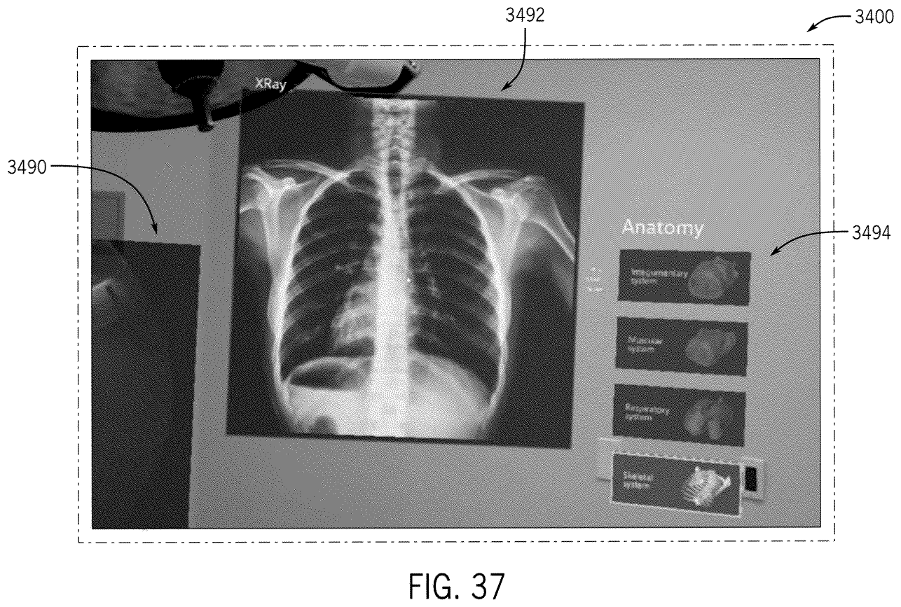

As an example, FIG. 32 illustrates a screen shot 2900 of an exemplary GUI display of the smartglasses 106 showing a menu 2980 of possible authorized participants with the option to select an account and login by gazing at the desired participant and using the voice command “Select” or similar. As yet another example, FIG. 33A illustrates a screen shot 3000 of an exemplary welcome GUI display of the smartglasses 106 showing an interface 3084 with an option to find people to call or to show a history of calls by gazing at either “Team” or “Call Log” and saying “Team” or “Call Log”. As yet another example, FIG. 33B illustrates a screen shot 3000′ of an exemplary GUI display of the smartglasses 106 showing an interface 3084′ with a list of people to call and/or view their profiles by gazing at the desired person and saying, for instance, “Call” or “See Profile.” As yet another example, FIG. 37 illustrates a screen shot 3400 of an exemplary smartglasses GUI display showing enlargement of an X-ray virtual object 3492 through, for instance, a voice command.

In any instance, the smartglasses wearer can say the command “Back”, “Home”, etc. to exit to another menu or display. Moreover, in one example, the smartglasses wearer can log out of the system by simply removing the smartglasses (detected, for instance, by one or more of the real-world environment sensors 140).

As can be appreciated, the virtual and actual input sensors 142 and 148 (as well as the real-world environment sensors 140 is some instances) allow for touch-free use of the smartglasses 106 that is not only convenient and efficient but also maintains sterility in an operating room of the healthcare facility 102.

As shown in FIG. 3B, the smartglasses application 134 may further include a mixed reality module 164 configured to cause display of superimposed images on a display screen of the smartglasses 106 to create a mixed-reality experience for the user. For instance, the mixed reality module 164 may cause display of images representing a navigation interface for the smartglasses 106, such as a main menu, account information (such as login information, settings, etc.), a call center (for connecting to remote users), a medical document/image library interface, etc. The mixed reality module 164 may cause display of feedback data sent from a remote user 116 (through the remote client computing device 118) to the smartglasses wearer 104, such as annotations, notes, medical images, etc. The mixed reality module 164 may also cause display of data requested by the smartglasses wearer 104, such as medical records, medical images, 3-D anatomy models, etc.

The smartglasses application 134 may further include an AR input module 168 configured to capture the virtual or actual sensor input data initiated by the smartglasses wearer 104, such as audio or touch input. In that regard, the AR input module 168 may capture virtual or actual sensor input data as audio files (e.g., dictation or commands), annotations of images displayed on the display screen of the smartglasses 106, user authentication data, remote user communication selection, gesture inputs, etc. For instance, the AR input module 168 may be in communication with the first and second speaker/microphones 130a and 130b on the smartglasses 106 to capture dictation files, which may be sent to a network device and uploaded to a patient's medical file or electronic health record (EHR). The AR input module 168 may also process speech commands to allow the smartglasses wearer 104 to navigate through various prompts, tools, menus, etc., displayed on the display screen of the smartglasses 106. For instance, the smartglasses wearer 104 may use the command “enlarge” to enlarge a virtual object when looking at that object (see the enlarged X-ray 3492 shown in the exemplary screen shot 3400 of FIG. 37).

As noted above, the AR operating system 132 further includes a smartglasses management application 136. The smartglasses management application 136 is configured to update the AR operating system 132 and/or the smartglasses application 134 (or other software or firmware of the smartglasses computing device 119) either automatically or in response to an input, push notification, etc. The smartglasses management application 136 may communicate with a device management application 216 on the application computing system 122 or another network device for receiving device updates. The smartglasses management application 136 may also send diagnostic information to the device management application 216 for monitoring the smartglasses computing device 119. In that regard, the smartglasses management application 136 may automatically receive updates from the device management application 216 in response to the sent diagnostic information.

The smartglasses computing device 119 may further include a processor 172 and memory 174 (including software/firmware code (SW) 176), an optional battery 177, and an input/output (I/O) controller 178. The input/output (I/O) controller 178 may be configured to connect the smartglasses computing device 119 to one or more devices, such as a handheld controller 131, a wireless keyboard/mouse, etc. All of the components of the smartglasses computing device 119 may communicate, directly or indirectly, with one another via one or more buses 180.

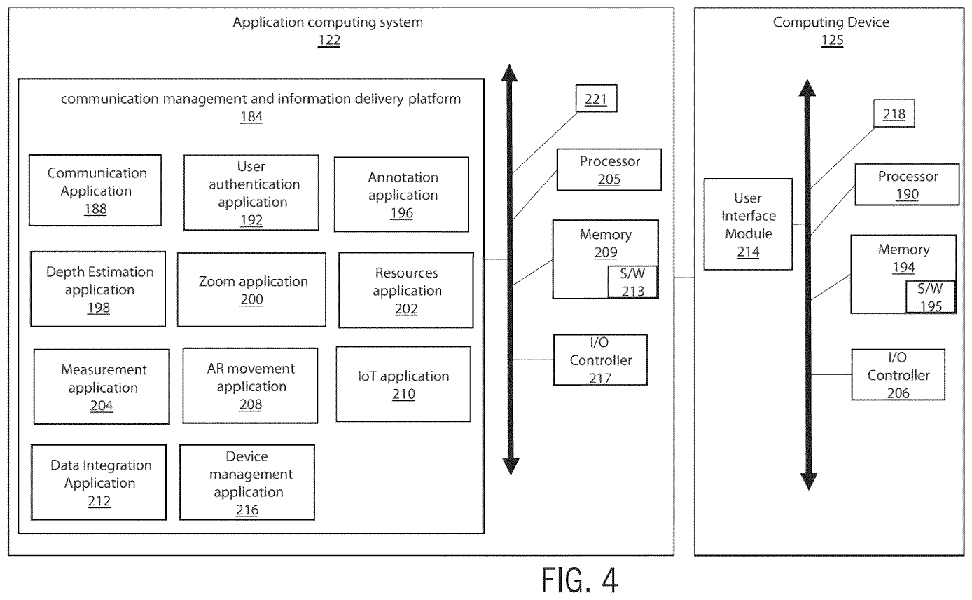

FIG. 4 depicts an exemplary block diagram of the application computing system 122 referenced above with respect to FIG. 1. In general, the application computing system 122 includes a communication management and information delivery platform 184 configured to support the communication and interaction between the smartglasses 106 and the remote client computing device 118 or any other computing devices on the network 120. In that regard, the communication management and information delivery platform 184 may support a communication application 188, a user authentication application 192, an annotation application 196, a depth estimation application 198, a zoom application 200, a resources application 202, a measurement application 204, an AR movement application 208, an IoT application 210, a data integration application 212, and a device management application 216.

The application computing system 122 may further include a processor 205 and memory 209 (including software/firmware code (SW) 213) and an input/output (I/O) controller 217. The input/output (I/O) controller 217 may be configured to connect the application computing system 122 to one or more devices, such as the computing device 125 (also shown in FIG. 4). All of the components of the application computing system 122 may communicate, directly or indirectly, with one another via one or more buses 221. The application computing system 122 may be made up of multiple servers, each running one or more of the applications noted above.

The applications of the application computing system 122 may be Web-based platforms (for example, cloud platforms) capable of being accessed over the network 120. In other example embodiments, a remote plug-in that uses cloud-based APIs (Application Program Interfaces) may be utilized to connect and extract the information from the application computing system 122. In any event, the cloud platforms are individualized to a specific group of users. For instance, a new or separate cloud platform is used for each hospital, medical group, etc., to ensure patient privacy and compliance with HIPAA requirements.

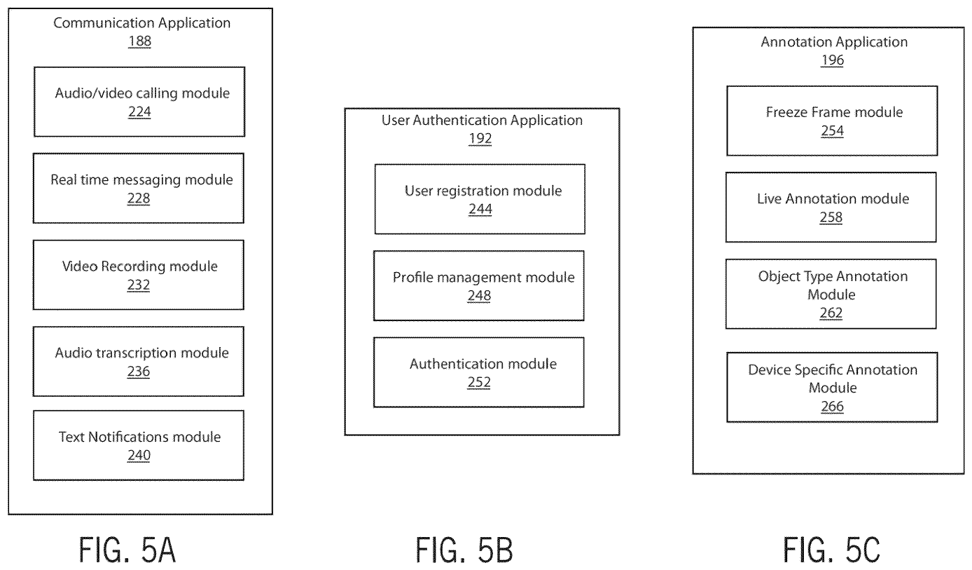

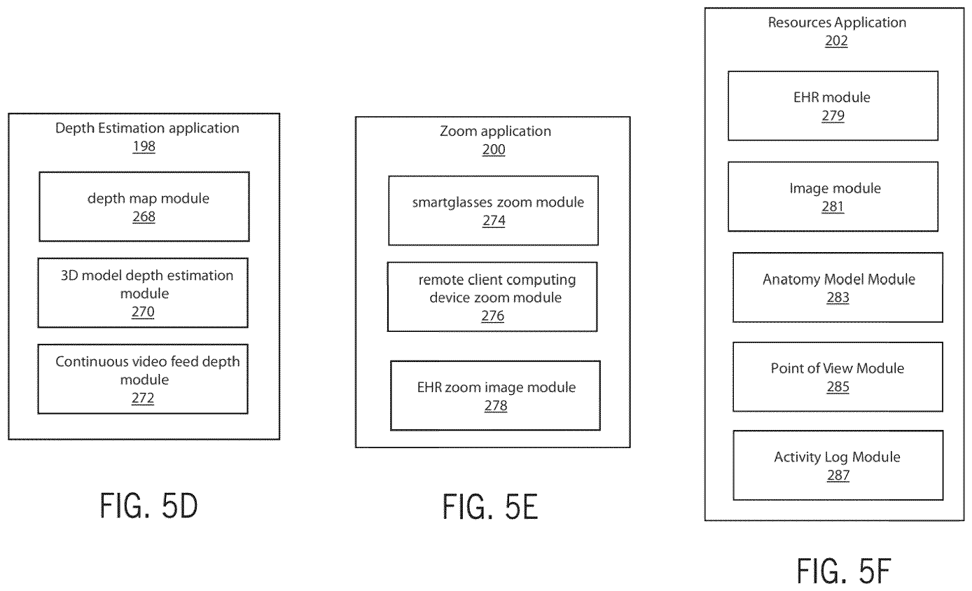

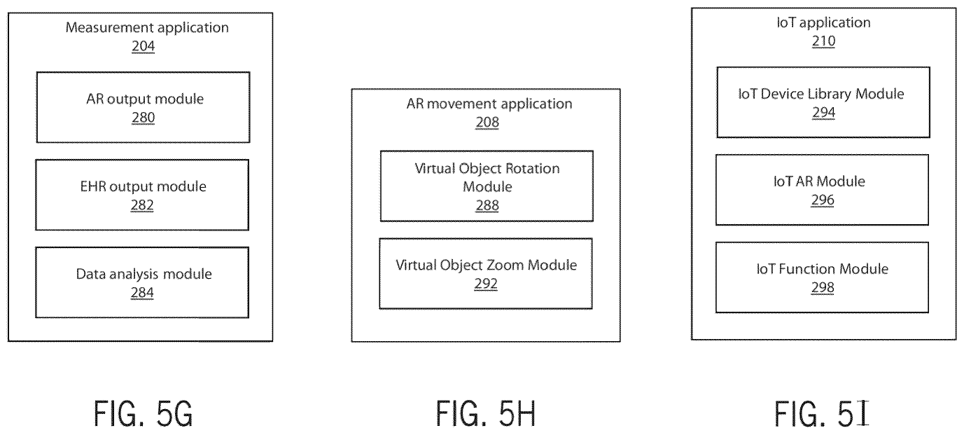

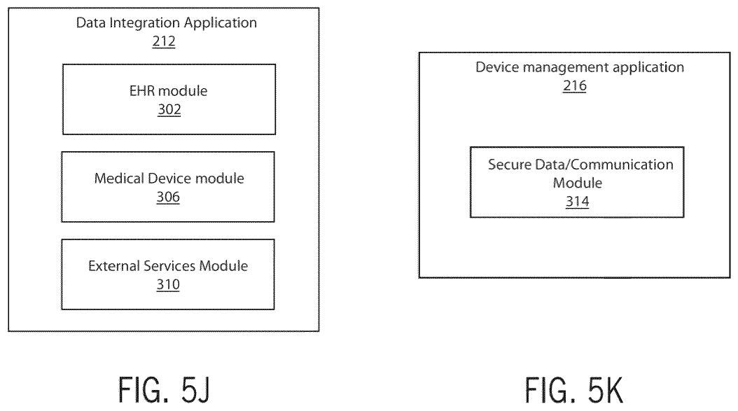

Referring additionally to FIGS. 5A-5K, exemplary embodiments of the applications of the application computing system 122 will now be described.

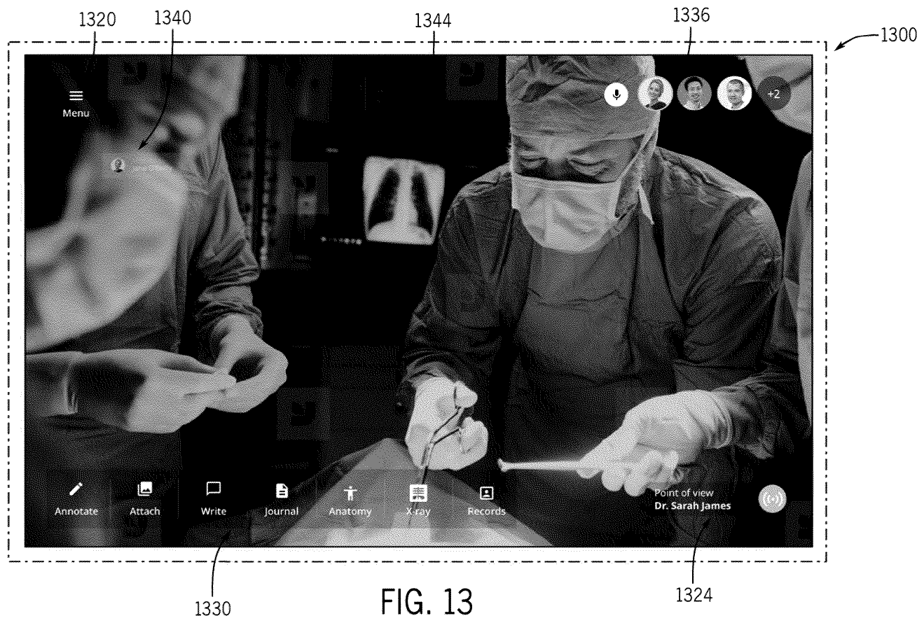

Referring first to FIG. 5A, the communication application 188 will first be described. The communication application 188 is generally configured to enable audio, video, and/or text communications between the smartglasses 106 and the remote client computing device 118 or any other computing devices on the network 120. In that regard, the communication application 188 includes an audio/video calling module 224 configured to allow two-way audio communication between the smartglasses 106 and the remote client computing device 118 and one-way video communication between the smartglasses 106 and the remote client computing device 118. Audio communication may optionally be initiated by the remote user 116 using a command, such as clicking a microphone button (see the microphone button in the screen shot 1300 of FIG. 13).

Regarding the one-way video communication, the audio/video calling module 224 is configured to send video captured from the medical multimedia data capture module 138 of the smartglasses application 134 to the remote client computing device 118. In that regard, the smartglasses wearer 104 broadcasts video and audio to an interface of the remote client computing device 118, and the user of the remote client computing device 118 broadcasts audio to the smartglasses wearer 104. The audio/video calling module 224 may be any suitable streaming service that enables secure, HIPAA-compliant communications, such as Agora.io RTC.

The communication application 188 further includes a real time messaging module 228 configured to allow two-way messaging communications between the smartglasses 106 and the remote client computing device 118. For instance, the real time messaging module 228 may be configured to support participant presence information (e.g., online or offline), invite participants to a video/audio call, reject video/audio calls, send annotation data, etc. The real time messaging module 228 may be any suitable streaming service that enables secure, HIPAA-compliant communications, such as Agora.io RTM.

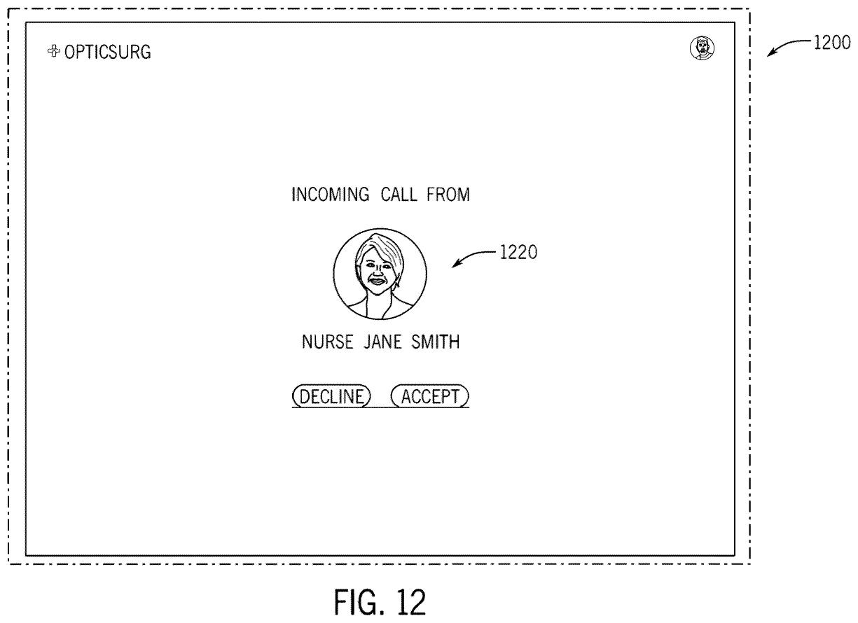

The audio/video calling module 224 and real time messaging module 228 of the communication application 188 may be implemented on the smartglasses 106 and remote client computing device 118 in any suitable manner. As non-limiting examples, FIG. 12 illustrates a screen shot 1200 of an exemplary GUI display of the remote client computing device 118 showing an incoming call interface 1220. FIG. 33A illustrates a screen shot 3000 of an exemplary welcome GUI display of the smartglasses 106 showing an interface 3084 with an option to select “Team” to find people to call and an option to select “Call Log” to show a history of calls. FIG. 33B illustrates a screen shot 3000′ of an exemplary GUI display of the smartglasses 106 showing an interface 3084′ with a list of people to call and/or view their profiles after selecting “Team” (see FIG. 33A), where the available people to call/view may be sorted by a category. As a non-limiting example, the people to call/view may be sorted by status (e.g., all, online, offline), role (e.g., all, doctor, nurse), and/or department (e.g., all, radiology, oncology, rehab). As another option, a group of people may be called by selecting “Group Call.” FIG. 33C illustrates a screen shot 3000″ of an exemplary GUI display of the smartglasses 106 showing a call log interface 3084″ for the smartglasses 106 after selecting “Call Log” (see FIG. 33A). FIG. 33D illustrates a screen shot 3000′″ of an exemplary GUI display of the smartglasses 106 showing an interface 3084″′ for calling a selected participant.

Other aspects of the communication application 188, including the audio/video calling module 224 and real time messaging module 228, may be implemented in any other suitable manner for supporting the communication session between the smartglasses 106 and the remote client computing device 118 or any other computing devices on the network 120.

The audio/video calling module 224 and the real time messaging module 228 may send data to a cloud-based analytics platform that is configured to provide a history of the audio/video communication, including ongoing calls. For instance, the cloud-based analytics platform may provide information about the user id, call quality and network conditions, etc. Information generated by the cloud-based analytics platform may be accessed through a web application interface supported by the provider(s) of the audio/video calling module 224 and the real time messaging module 228.

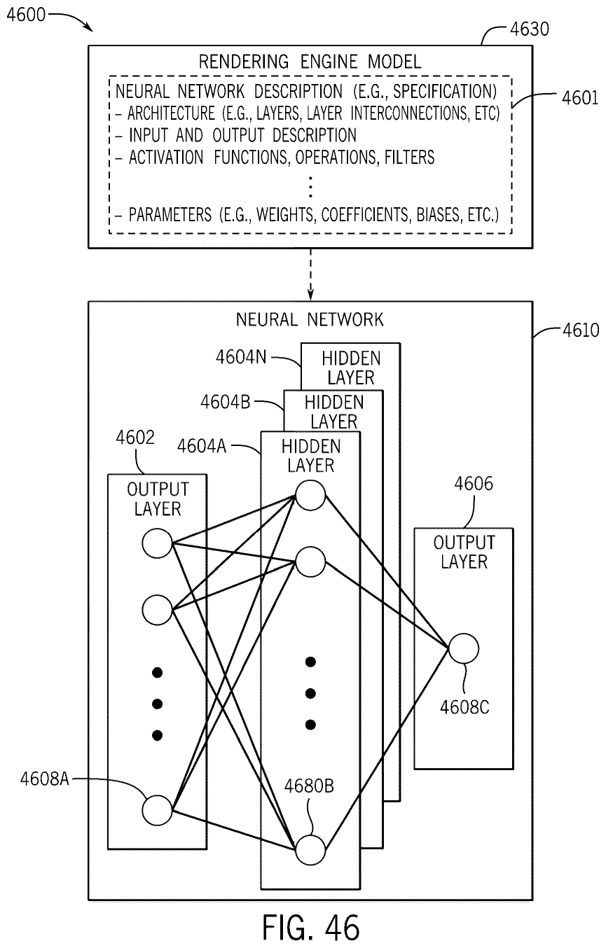

The communication application 188 may further include a video recording module 232 configured to capture video input of the smartglasses 106 (through the one or more cameras) for use in a downstream application or service. For instance, video input captured by the smartglasses 106 may be used by remote users after the medical procedure (i.e., not in real time) for training purposes. In another instance, video input captured by the smartglasses 106 may be used by cloud-based artificial intelligence (AI) platforms for processing certain aspects of the AR or remote user experience, such as identifying objects and/or calculating the depth of objects viewed by the smartglasses user, for anomaly detection, etc. For instance, the AI platform(s) described with respect to FIG. 46 may be used for processing certain aspects of the AR or remote user experience.

The communication application 188 may further include an audio transcription module 236 configured to process audio captured by the medical multimedia data capture module 138 for sending to the remote client computing device 118 and/or the healthcare facility computing system 124 as text. In that regard, the audio transcription module 236 is configured to receive the audio data and process the data (i.e., transcribe to text) for sending as text data. For instance, the audio transcription module 236 may package and send the audio-to-text data to the remote client computing device 118 such that a user can see, in real-time, the text data (e.g., an annotation on the video, a note made to a file, etc.).

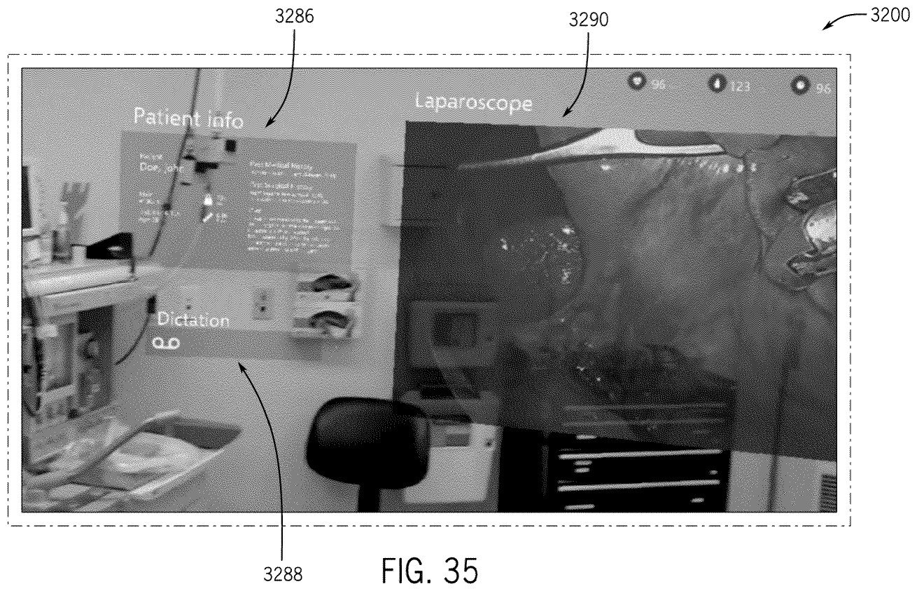

In another example, the audio transcription module 236 may package and send the audio-to-text data to the healthcare facility computing system 124 for updating an electronic health record (EHR) of the patient. As a non-limiting example, FIG. 35 illustrates a screen shot 3200 of an exemplary GUI display of the smartglasses 106 showing a dictation button represented as a virtual image 3288. The smartglasses wearer 104 may select the dictation button to send for instance, an audio-to-text data to the healthcare facility computing system 124 for updating an electronic health record (EHR) of the patient.

The communication application 188 may further include a text notifications module 240 configured to, for instance, send text notifications or messages to offline users. The text notifications module 240 may be used to indicate an incoming call to an offline user through a text notification/message. The text notifications module 240 may be any suitable messaging service that enables secure, HIPAA-compliant communications, such as Twilio programmable text service.

Referring to FIG. 5B, the user authentication application 192 of the communication management and information delivery platform 184 will now be described. The user authentication application 192 may include a user registration module 244 configured to, for instance, facilitate registration of the smartglasses wearer 104 and the remote users (such as remote user 116 of the remote client computing device 118). In one example, the user registration module 244 may enable a new smartglasses wearer 104 or remote user 116 to register a profile, including a login id, a password, a profile picture, a cellular number (e.g., for text notifications), etc. The user authentication application 192 may further include a profile management module 248 configured to manage and store the profile of the registered users, reset passwords, etc., and an authentication module 252 configured to facilitate login of the smartglasses wearer 104 and the remote users 116.

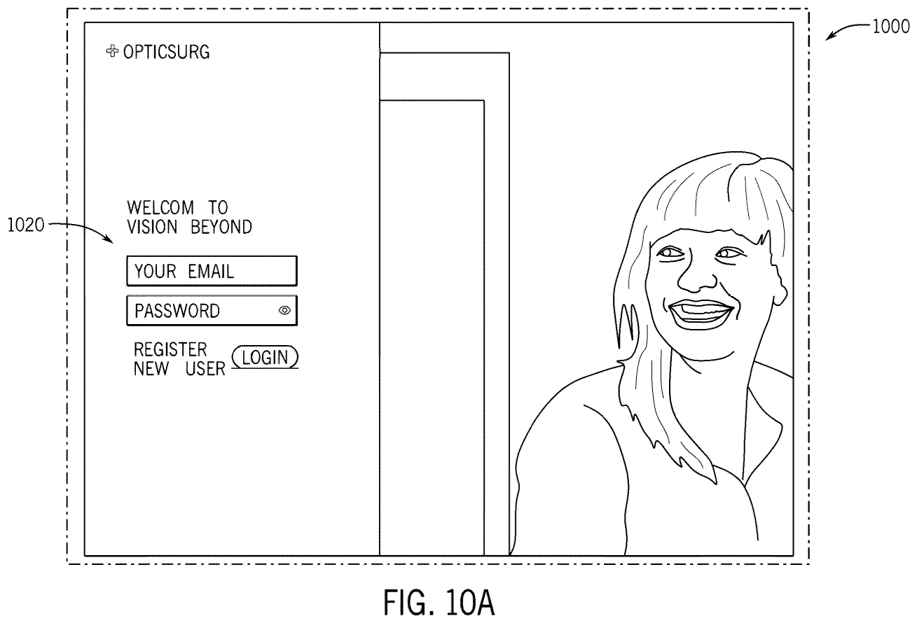

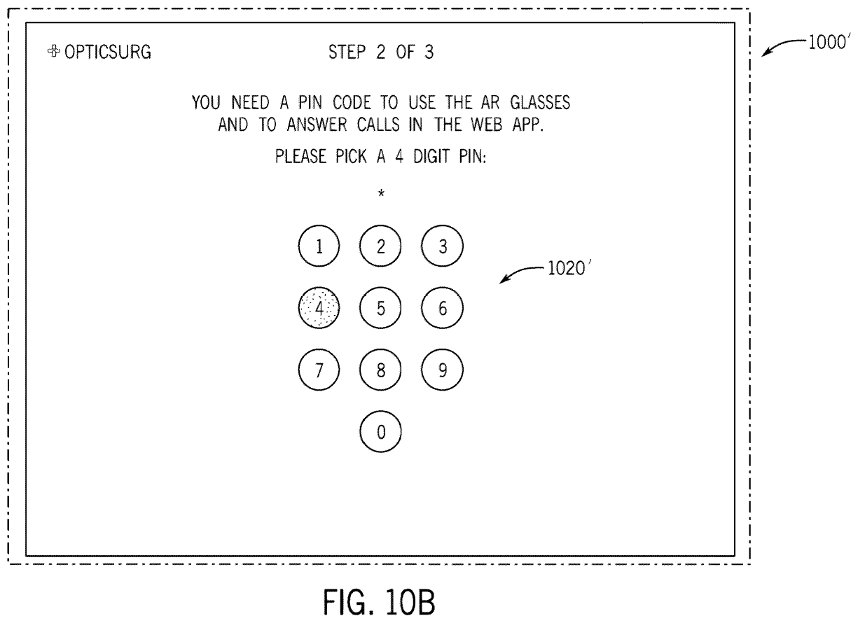

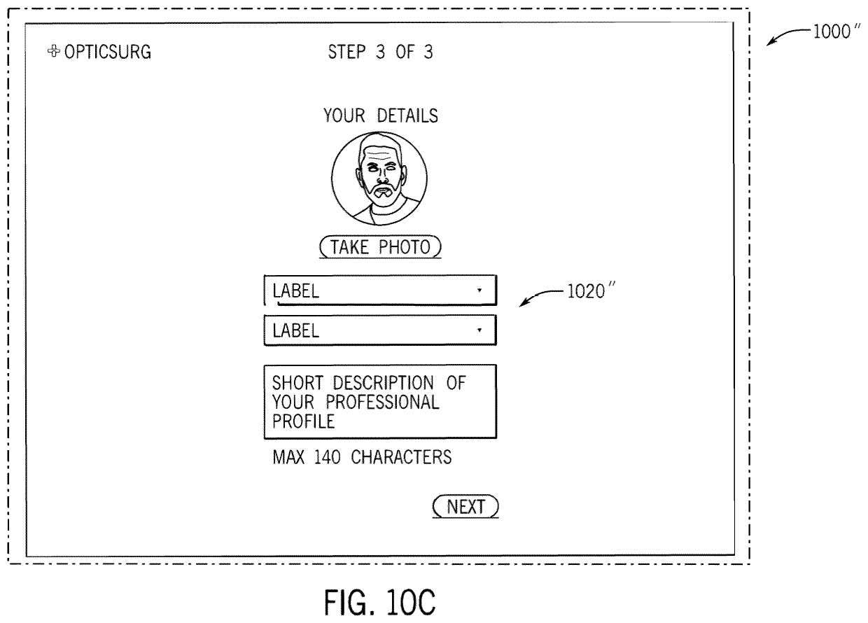

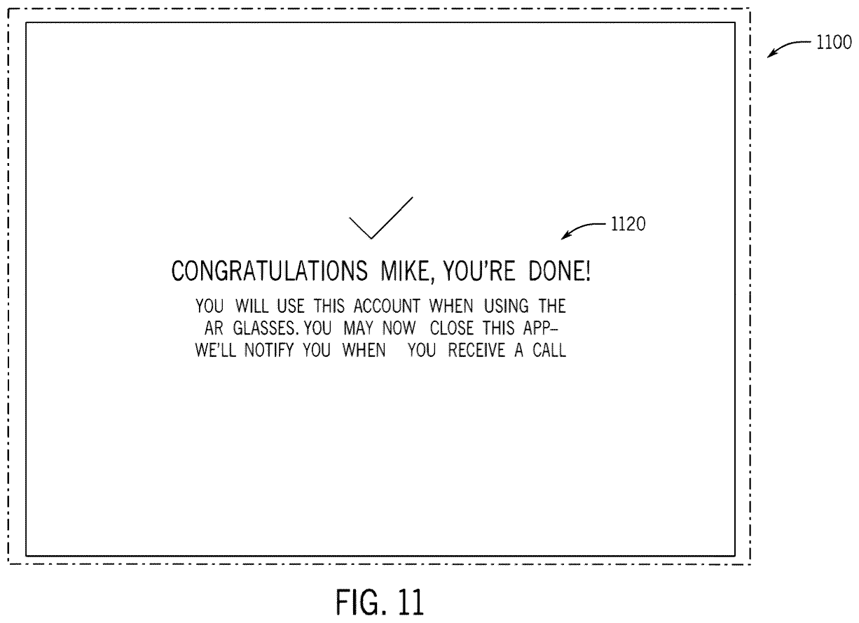

As non-limiting examples, FIGS. 10A-10C illustrates screen shots of an exemplary GUI display of the remote client computing device 118 showing a login/registration interface 1020. For instance, FIG. 10A illustrates a screen shot 1000 of an exemplary GUI display of the remote client computing device 118 showing a login/registration interface 1020, where a user may register an account with an email and password and then login to the account to use the system. FIG. 10B illustrates a screen shot 1000′ of an exemplary GUI display of the remote client computing device 118 showing a pin code registration interface 1020′, where a user may create a pin code for using the smartglasses 106 and answering calls from a smartglasses wearer in a web application on the remote client computing device 118. FIG. 10C illustrates a screen shot 1000″ of an exemplary GUI display of the remote client computing device 118 showing an interface 1020″ for creating a user profile, where a user may provide information about themselves that will be available/visible to other users of the system. FIG. 11 illustrates a screen shot 1100 of an exemplary GUI display of the remote client computing device 118 showing an interface 1120 indicating completion of user profile registration.

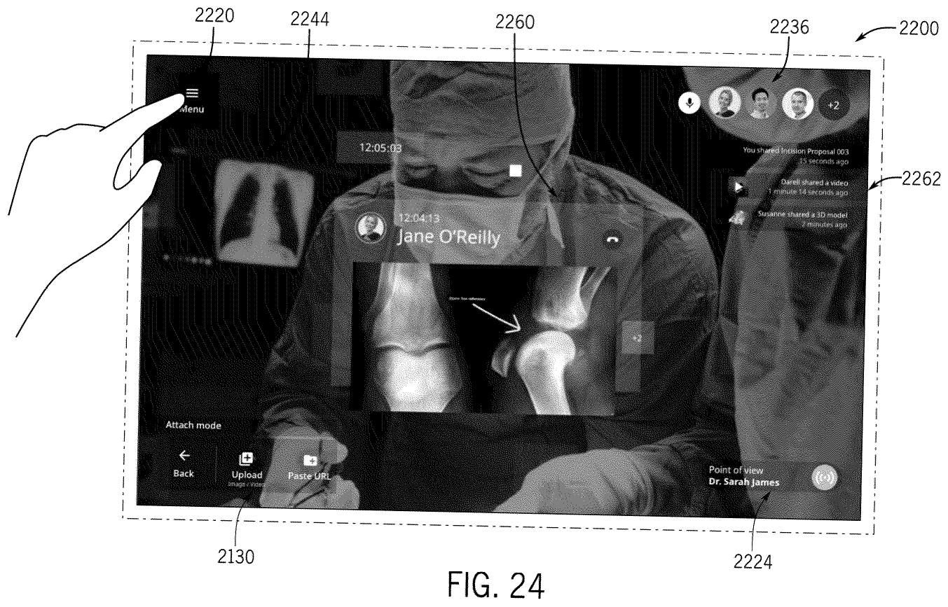

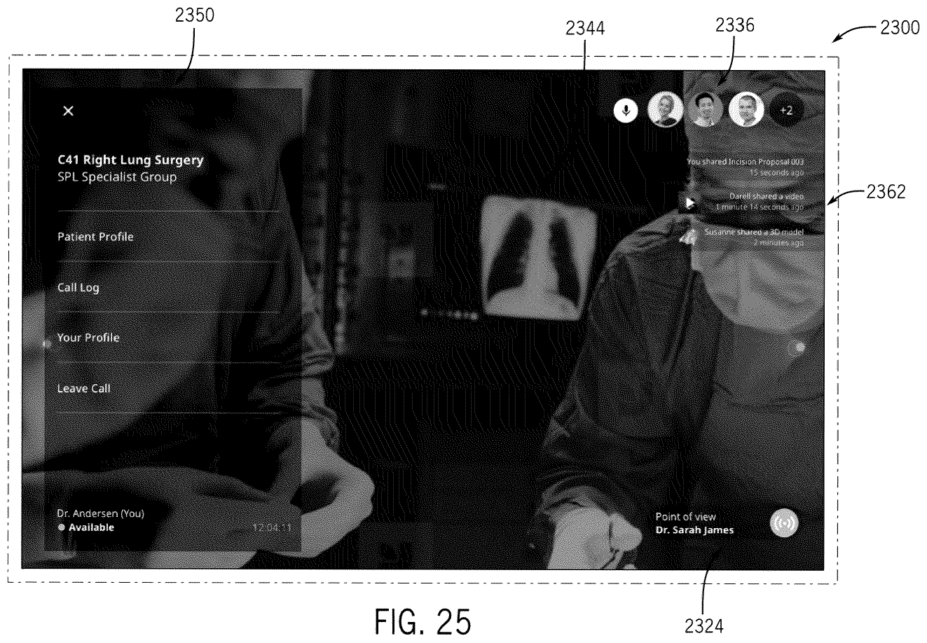

FIG. 25 illustrates a screen shot 2300 of an exemplary GUI display of the remote client computing device 118 showing a drop-down menu providing access to profile information represented as an image 2350. The drop-down menu 2350 may be initiated from a top-level menu. For instance, FIG. 24 illustrates a screen shot 2200 of an exemplary GUI display of the remote client computing device 118 showing a top-level menu represented as an image 2220.

In further aspects, FIG. 32 illustrates a screen shot 2900 of an exemplary GUI display of the smartglasses 106 showing a menu 2980 of possible authorized participants with the option to select an account and login. Moreover, FIG. 13 illustrates a screen shot 1300 of an exemplary GUI display of the remote client computing device 118 showing profile picture icons 1336 of all participants in the communication session. Other aspects of the user authentication application 192 may be implemented in any other suitable manner for supporting the registration, management, and login of smartglasses wearers 104 and/or remote users 116.

In another aspect, the authentication module 252 may facilitate login when the smartglasses wearer 104 scans a QR code with a camera on the smartglasses 106 shown, for instance, on a secondary device, such as a smartphone or tablet. In this regard, the smartglasses wearer 104 can login without excessive use of voice commands or gestures, which can sometimes be cumbersome and time-consuming.

The user authentication application 192 may be any suitable profile management and authentication service that enables secure, HIPAA-compliant data management for each group of users (i.e., a new or separate service is used for each hospital, medical group, etc., to ensure patient privacy), such as Google Cloud Identity, Google Firebase (and specifically, such as Google Firebase Authentication, Google Firebase Cloud Store, Google Firebase Storage, and Google Firebase Cloud Functions).

Referring to FIG. 5C, the annotation application 196 of the communication management and information delivery platform 184 will now be described. The annotation application 196 is generally configured to capture annotation information, indicators, data, or another AR object inputted by the remote user 116 on the remote client computing device 118 and transmit or push that data from the remote client computing device 118 to the smartglasses 106, as generally described in U.S. Patent Application Publication No. 2020/0234809, entitled “Method and system for optimizing healthcare delivery,” the entire disclosure of which is hereby incorporated by reference herein.

The annotation data may be used, for instance, to instruct or guide the medical practitioner on care of the patient associated with the general care of the patient. More specifically, the remote user 116 of the remote client computing device 118, when viewing medical multimedia data captured by the medical multimedia data capture module 138, may input annotations or other indicators that are pushed to the smartglasses 106 via the network 120 for display within the display device of the smartglasses 106. In this manner, the remote user 116 of the remote client computing device 118 may provide instructions and/or feedback in the form of annotations, indicators, etc., that may guide the smartglasses wearer 104 through one or more medical procedures.

The remote user 116 may initiate an annotation by selecting an option in a menu displayed on the display of the remote client computing device 118. For instance, FIG. 13 illustrates a screen shot 1300 of an exemplary GUI display of the remote client computing device 118 showing a menu represented as an image 1330. The remote user 116 may select an option from the menu 1330 (e.g., annotate, attach, write, journal, anatomy, X-ray, or records) to annotate one of the available resources.

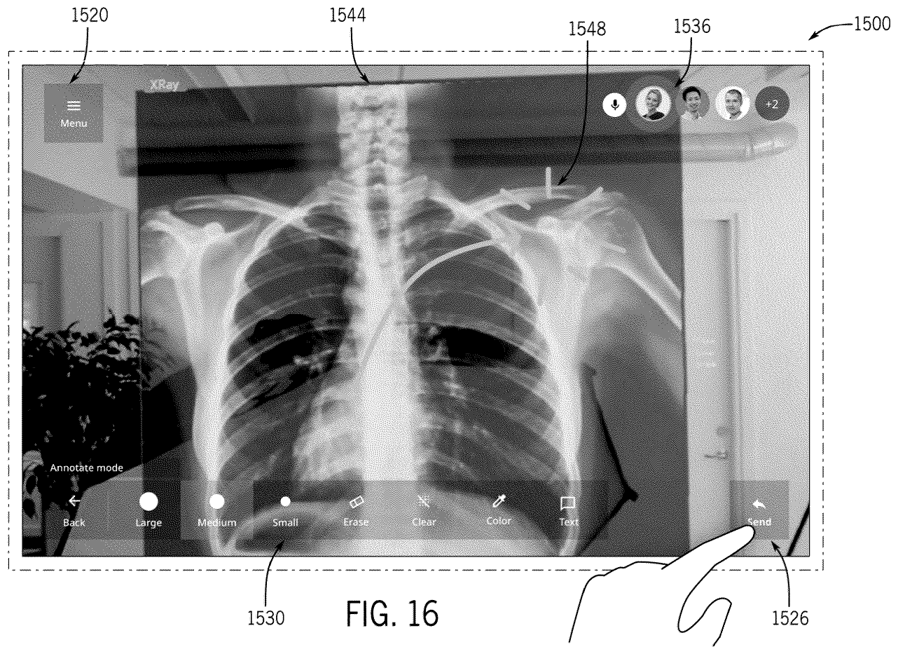

Once an annotation action is initiated, the remote user 116 can create a desired annotation using an annotation menu. For instance, FIG. 16 illustrates a screen shot 1500 of an exemplary GUI display of the remote client computing device 118 showing an annotation menu represented as an image 1530. The remote user 116 may select an option from the menu 1530 (e.g., large, medium, small, erase, clear, rotate, color, text) to create a desired annotation. Similar menus After making the annotation, the remote user 116 may click on a “send” button to send the annotated image to, for instance, the smartglasses wearer 104. For instance, FIG. 16 shows a send button represented as an image 1526.

The annotations may be configured to support the specific application of the communication management and information delivery system 100. For instance, in the exemplary embodiments described herein, different annotation types may be used for efficient communication between the remote user 116 and the smartglasses wearer 104, such as arrows, hotspots, and freehand drawings. Annotations can be sticky and stay visible until removed by the user or ephemeral and only shown for a certain time or until the user looks at them. Moreover, each remote user participant (i.e., each remote user using a unique remote client computing device 118) may be assigned a unique color that is used for annotations. The uniquely assigned color may be presented together with the name and photo of the remote user (see the profile picture icons 1336 shown in FIG. 13) such that when the annotations are presented in the color of the creator, everyone on the call will know who added each annotation. The uniquely assigned color tied to the name and photo of the remote user may be assigned by the user authentication application 192, which may communicate with the annotation application 196 to assign the same color for the annotations of that user.

It can be appreciated that clinicians who are working in a fast-paced environment are moving quickly and making fast decisions. Accordingly, annotations must quickly appear on the display of the smartglasses 106 to be effective. Prior art systems typically use a “freeze frame” method, where the frame of the video feed (i.e., the real-time medical multimedia data feed) is frozen and the remote user annotates that frame. In other words, the display of the remote client computing device 118 would provide the remote user 116 with a snapshot of what the smartglasses camera records, and in that fixed frame the remote user 116 adds their annotations. These annotations are submitted back to the glasses user and displayed as graphics at the 3-D location inferred by the remote user's annotation. This “freeze frame” method can cause a significant lag (e.g. 5-7 seconds) to show up on the display of the smartglasses 106. In such an instance, the annotations are typically not used, and the remote user will instead use their voice.

The annotation application 196 is configured to quickly deliver the remote user annotations on the display of the smartglasses 106 using at least one of various modules. As can be seen in FIG. 5C, the annotation application 196 may include a freeze frame module 254, a live annotation module 258, an object type annotation module 262, and/or a device specific annotation module 266.

The freeze frame module 254 may be configured to add or deliver annotations on the display of the smartglasses 106 by freezing a video feed frame displayed on the on the display of the remote client computing device 118 of the remote user 116. Once frozen, the remote user annotates the frame, and the annotation is sent back to the smartglasses 106 and displayed as graphics on the display. The freeze frame module 254 improves over prior art “freeze frame” methods by decreasing the latency between the annotation and the delivery of the graphics on the smartglasses display.

In order to decrease the latency or create a “fast annotation” system, the freeze frame module 254 may use simple annotations that can be quickly and easily displayed on the smartglasses display. For instance, the annotations may be 2-D graphics that are not mapped to any depth of an object viewed by the smartglasses wearer 104. Rather, the annotation may simply correspond to x-and y-coordinates of a mouse click or touch screen input on the frozen video frame of the remote user. In that regard, the annotation may present as a “firework”, a pulsing dot, a circle, or any other suitable virtual marker in the glasses field of view that corresponds to the 2-D location of the remote user's click/touch. In another example, the annotation may present as a peripheral arrow to nudge the smartglasses wearer 104 to look in a certain direction (e.g., “look down”, “look left”, etc.). To create such a directional annotation, the remote user may (optionally after selecting the directional annotation from a menu of possible annotation types) click/touch on a certain region of the frozen video frame. In the alternative, the directional annotation may be created by using arrow keys on a keyboard or through voice commands.

In another instance, the freeze frame module 254 may use simple annotations that can be quickly and easily displayed on the smartglasses display, but may also use the processing capabilities of the depth estimation application 198 to display the annotation at the 3-D location inferred by the remote user's click/touch. In that regard, the 3-D graphics may be mapped to a depth of an object viewed by the smartglasses wearer 104. The 3-D graphics may present as a simple annotation described above (e.g., a firework, laser pointer, arrow, etc.), or may instead present as a freehand symbol, indication, mark, or drawing. For instance, a remote user may circle an object on the frozen screen, such as equipment, a body part/area, etc., together with an arrow or other marking to indicate a recommended movement or action for the smartglasses wearer 104.

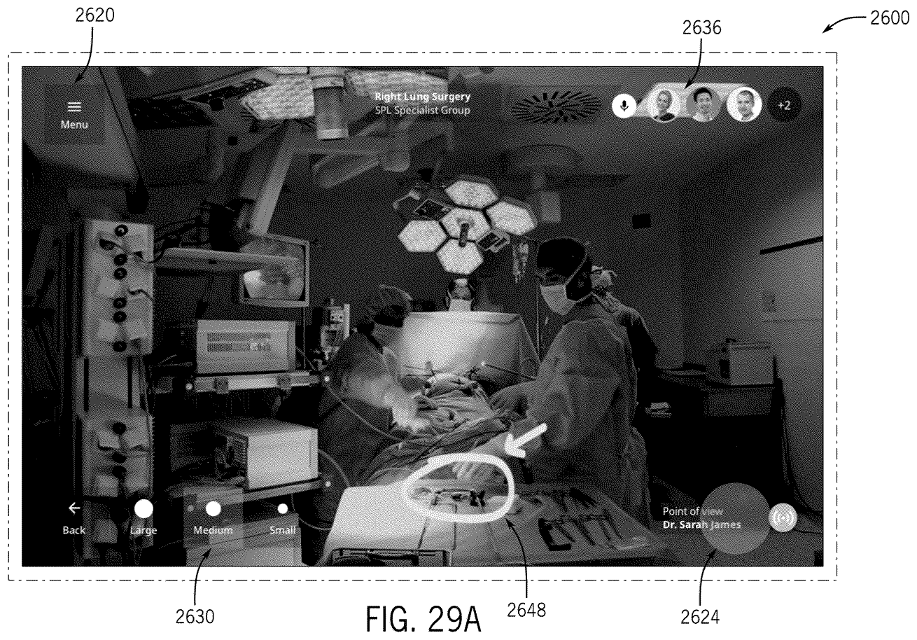

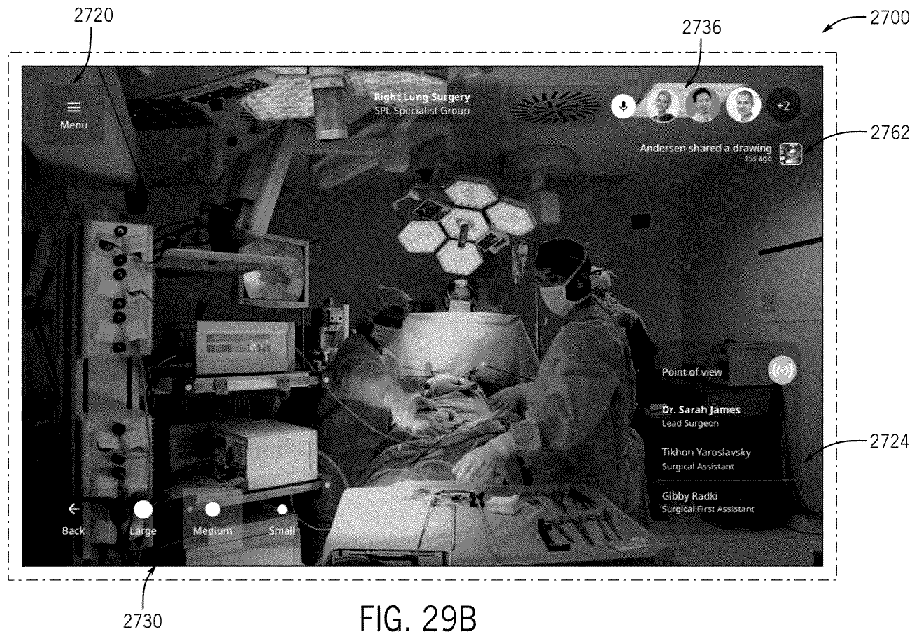

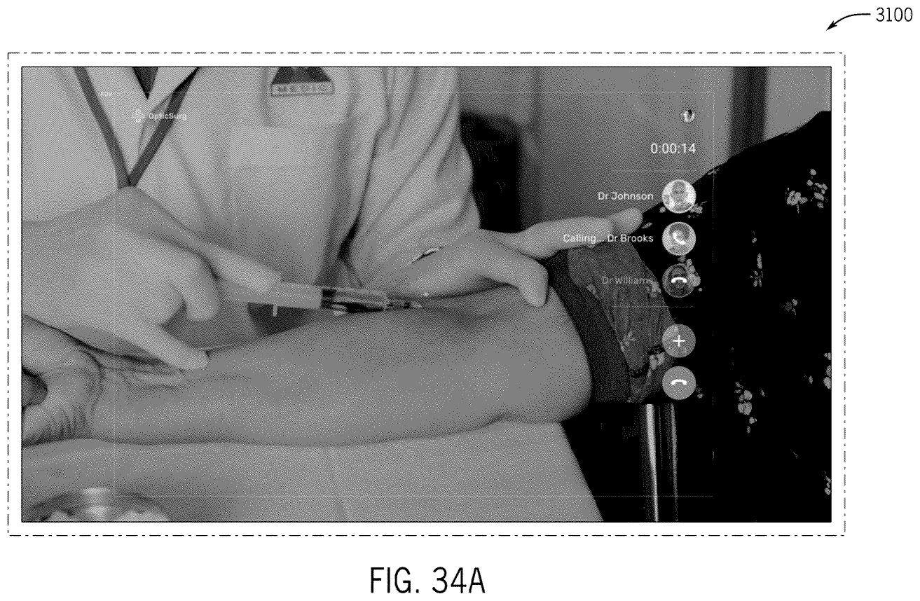

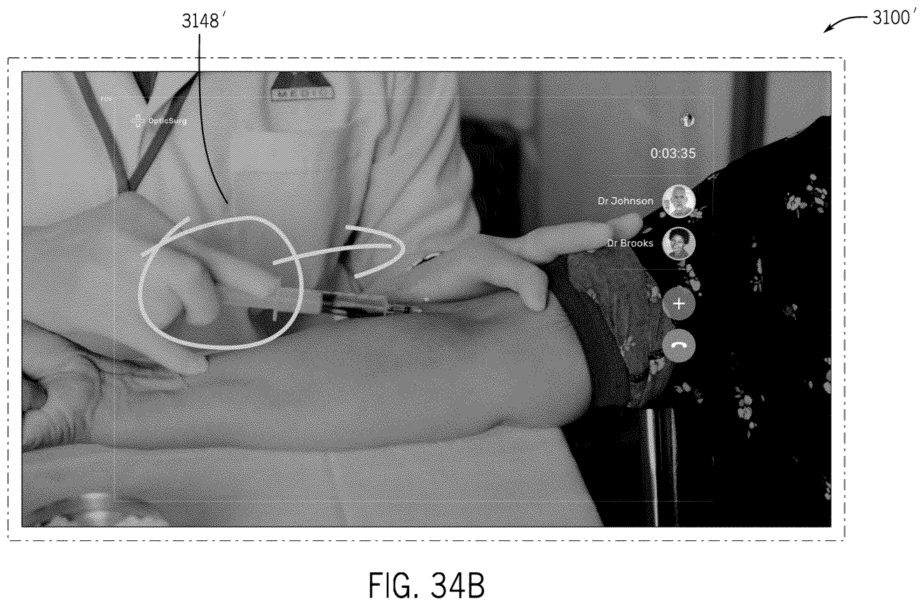

FIG. 29A illustrates a screen shot 2600 of an exemplary remote client computing device GUI display showing a freehand annotation displayed as an electronic image 2648 that was added by the remote user 116. FIG. 34B illustrates a screen shot 3100′ of an exemplary GUI display of smartglasses 106 showing a freehand annotation displayed as a virtual image 3148′ that was added by a remote user 116 (after the remote user 116 was added to the communication session, as shown in progress in the screen shot 3100 of an exemplary GUI display of the smartglasses 106 shown in FIG. 34A). It should be appreciated that the freehand annotation displayed in FIGS. 29A and 34B may have instead been added by the smartglasses wearer.

The fast annotation may play for a predetermined period of time before fading or disappearing, such as 0.5 seconds. In the alternative, the annotation may play until removed by the smartglasses wearer 104, such as by audio command. The fast annotation system decreases the latency between the annotation and the delivery of the graphics on the smartglasses display from a traditional “freeze frame” method by at least 4-5 seconds, e.g., from 5-7 seconds to about 1 second.

As noted above, the annotation application 196 may also include a live annotation module 258. The live annotation module 258 may be generally configured to provide another implementation or option for providing a “fast annotation” system to support the many situations where the speed of communication is key. In that regard, the live annotation module 258 can be used to produce a live annotation corresponding to a remote user's click/touch.

More specifically, a remote user can click/touch a live video feed to reference a spot for viewing by the smartglasses wearer 104. The annotation can be presented in the display of the smartglasses 106 until the remote user releases her finger or the pointing device used. In the alternative, the annotation can be ephemeral and begin fading immediately after the remote user releases his/her click/touch. The live feed annotation can be a simple annotation to support the speed of the live video feed, such as the annotations described above with reference to the freeze frame module 254 (either as 2-D or 3-D graphics).

As noted above, the annotation application 196 may also include an object type annotation module 262. The object type annotation module 262 may be generally configured to automatically provide an annotation having a type, category, and/or appearance that corresponds to the object being annotated. In one example, if the object is a human, the annotation type might default to a soft annotation (a firework, laser pointer, etc.) and/or follow the movement of the human. In another example, if the object is a button on a machine, the annotation type might default to an arrow with a push animation.

The annotation application 196 may use AI/ML platforms for identifying objects viewed by the smartglasses 106. AI/ML algorithms may be run on the smartglasses computing device 119, on a cloud service, and/or on the remote client computing device 118 to detect and identify objects in the frozen or live video feed frame and to select an annotation type accordingly.

In another example, the object type annotation module 262 may provide annotation menus, toolsets, etc., based on the current use or scene of the smartglasses wearer 104. The annotation menus, toolsets, etc., would correspond to the current use or scene of the smartglasses wearer 104, which may be automatically detected through sensors and/or use of AI/ML algorithms, or which may be selected as an input by the smartglasses wearer 104 and/or the remote user. For instance, the available annotation menus, toolsets, etc., might differ if the smartglasses wearer 104 is in a surgery rather than a medical device training situation or a telehealth setup. In that regard, the object type annotation module 262 may simplify user interaction as well as increase the speed of communication, which is vital in critical care use.

As noted above, the annotation application 196 may also include a device specific annotation module 266. The device specific annotation module 266 may be generally configured to automatically provide an annotation having a type and/or appearance that corresponds to a type or capabilities of a smartglasses device detected. In one example, if the smartglasses device is a high-end device having substantial processing capabilities, the annotation type might default to a 3-D graphic anchored to objects in the physical world viewed by the wearer. In another example, if the smartglasses device is a low-end device having lower processing capabilities, the annotation type might default to a 2-D graphic. For instance, the 2-D graphic may be pre-rendered on-top of the camera image for lower end smartglasses devices lacking spatial awareness capabilities.

Matching the annotation type to the capabilities of the smartglasses device can benefit the smartglasses device user experience, who may be medical experts supporting multiple frontline staff. The device specific annotation module 266 helps maintain simplicity and consistency between various types of smartglasses hardware, thus keeping focus on the issue at hand through a familiar video conference interface.

In order for a remote user to effectively place annotations and virtual markers on objects connected to the physical world viewed by the smartglasses wearer 104, it is beneficial if the communication management and information delivery system 100 has an understanding of the physical environment surrounding the smartglasses wearer 104. For instance, annotations and virtual markers can be more precisely placed on objects in the physical world if the system 100 can accurately determine the distance between the smartglasses wearer 104 and the object, i.e., the object depth.

A technique of estimating the distance to objects relative to the smartglasses wearer 104 is called depth estimation. With the use of depth estimation, an annotation, etc., may be mapped to a depth of an object viewed by the smartglasses wearer 104. Without the use of depth estimation, the annotation is positioned at a fixed distance from the smartglasses wearer 104. Hence, even though an annotation may initially appear to be positioned in a given place relative to objects in the real-world scene, it is in fact positioned in a very different place. This leads to a poor user experience and important usability challenges, since whenever the smartglasses wearer 104 moves, the real position of the annotation becomes apparent. More specifically, the annotation will start moving away from the place it should have been. As an example, if the remote user 116 draws a circle on a patient's leg and the smartglasses wearer 104 moves, suddenly the circle is no longer on the patient's leg but may be, perhaps floating in the air. The depth estimation application 198 of the communication management and information delivery platform 184 can be used to accurately determine the distance between the smartglasses wearer 104 and objects in the real-world scene.

Referring to FIG. 5D, the depth estimation application 198 will now be described. The depth estimation application 196 is generally configured to receive depth estimation data from the one or more depth estimation sensors 142, process the depth estimation data, and package the processed depth estimation data for sending to the remote client computing device 118 or another computing device on the network 120 (such as a cloud-based AI platform). As noted above, the one or more depth estimation sensors 142 are configured to capture depth estimation data used to estimate the depth of solid objects (e.g., the patient's anatomy, medical equipment, etc.) viewed by the smartglasses wearer 104. For instance, the first centered forward facing sensor assembly 128, which may include proximity and ambient light sensors and/or a red-green-blue (RGB) camera (a “color camera”), may be used to provide a live video feed of the real world scene. The first and second lateral forward facing sensor assemblies 129a and 129b, which may include first and second grayscale cameras (a stereo camera setup), may be used to track the position of the smartglasses 106.

In one aspect, the depth estimation application 198 is configured to produce a point cloud, i.e. coordinates in 3-D space where solid objects viewed by the smartglasses wearer 104 have been detected. The point cloud may be defined by a depth map or depth image, where a pixel value corresponds to the distance between the smartglasses 106 and the nearest solid object at that position. The depth map can be transformed to overlay a camera image containing color data (captured by the sensors of the smartglasses 106), which when combined defines an RGBD image. When annotations are added by a remote user viewing a live feed of RGBD images, the position of the annotation can be retrieved and mapped to objects in the physical world of the smartglasses wearer 104.

In a further aspect, the head pose of the smartglasses wearer 104, i.e. the orientation and position of the head worn smartglasses camera(s) at the moment the image was taken is used to further classify the 3-D position of the annotation. The position and orientation of the camera together with camera and lens parameters may be used in combination with the 2-D position of the annotation in the video feed image and the estimated distance from the camera to the nearest physical object at that specific 2-D position. These combined data points may be used to define the 3-D position where the annotation is positioned.

Depth may be estimated based only on images from the color camera or based on a combination of images from the first and second grayscale cameras and the color camera. For instance, the depth estimation application 198 may run a depth map module 268 configured to create a depth map based on a combination of the grayscale images and the color images. More specifically, after calculating a disparity map, the depth estimation application 198 creates a depth map using technology and methods well known in the art. The depth map is then combined with a depth map estimated by a machine learning algorithm based on the RGB image from the color camera. In one exemplary embodiment, the machine learning algorithm may be trained to create depth images based on direct input from the first and second grayscale cameras and the color camera.

In another instance, the depth estimation application 198 may run a 3-D model depth estimation module 270 configured to run a machine learning algorithm to create a continuous rough depth estimation of physical objects viewed by the smartglasses wearer 104. Over a period, the continuous rough depth estimation can be used to create a high quality 3-D model of the surroundings. The 3-D model depth estimation module 270 may instead run on the smartglasses computing device 119, and the remote service of the depth estimation application 198 may only be used if the current knowledge of what the smartglasses wearer 104 is looking at is too limited.

In another instance, the depth estimation application 198 may run a continuous video feed depth module 272 configured to run a machine learning algorithm to continuously estimate depth based on the video feed transmitted during a call with the remote client computing device 118. The depth of physical objects viewed by the smartglasses wearer 104 are estimated based on multiple frames as input.

It can be understood that depth is calculated using the continuous video feed depth module 272 as a “side-effect” of necessarily having a video stream from an ongoing call between the smartglasses wearer 104 and the remote user. However, the AR experience is enriched with the ability to add virtual elements (i.e., annotations and virtual markers) on objects viewed by the smartglasses wearer 104. Moreover, continuously estimating depth in this manner improves the quality of depth estimates. Nevertheless, it can be appreciated that such a continuous video feed method increases complexity and power consumption of the applied algorithms.

By using one or more of the depth estimation modules discussed above, the depth estimation application 198 can accurately estimate the depth of physical objects viewed by the smartglasses wearer 104. When using this depth estimation data, the annotation application 196 may accurately place annotations and virtual markers on objects viewed by the smartglasses wearer 104. In that regard, depth estimation may only be performed when a remote user 116 starts to add an annotation, virtual marker, etc. In this manner, the processing and power consumption of the communication management and information delivery platform 184 can be minimized to optimize system performance. Furthermore, the depth estimation application 198 may use all of the modules 268, 270, and 272 to perform a multi-level depth analysis for increasing depth accuracy, and/or the depth estimation application 198 may use only one of the modules 268, 270, and 272 based on the sensed real world surroundings to save processing and power consumption and optimize system performance.

The machine learning algorithms used by the depth estimation application 198 may be of any suitable type and language depending on platform or operating system, such as Unity Barracuda, PyTorch or Google Tensorflow. Moreover, it should be appreciated that in some embodiments, the depth estimation application 198 may be a module on the smartglasses application 134, rather than as part of the communication management and information delivery platform 184 of the application computing system 122.

Referring to FIG. 5E, the zoom application 200 of the communication management and information delivery platform 184 will now be described. The zoom application 200 is generally configured to process visual data signals captured by the zoom sensors 144 and output one or more visual data signals to produce a zoomed-in or zoomed-out image data for display on a computing device, such as the display screen of the smartglasses 106 or the remote client computing device 118. The zoom application 200 communicates with the depth estimation application 198 as needed to acquire depth data of any objects for accurately producing a zoomed-in image.

The zoom sensors 144 may output one or more signals, which may be processed by a smartglasses zoom module 274 for displaying a zoomed-in image on the display screen(s) of the smartglasses 106. In that regard, by using the smartglasses zoom module 274, a microscope is essentially integrated into the smartglasses 106. In addition or in the alternative, the zoom sensor signals may be processed by a remote client computing device zoom module 276 for displaying a zoomed-in image on the remote client computing device 118 for viewing by the remote user 116. In addition or in the alternative, the zoom sensor signals may be processed by a EHR zoom image module 278 for sending a zoomed-in image to the healthcare facility computing system 124 for storage in a EHR of the patient or other secure databases.

Referring to FIG. 5F, the resources application 202 of the communication management and information delivery platform 184 will now be described. The resources application 202 is generally configured to provide access to various electronic resources used to provide electronic content to support the procedure being performed by the smartglasses wearer 104. For instance, the resources application 202 may be used to access electronic patient records or data, electronic images (such as MRI, X-ray, or CAT scan images), 3-D anatomy models, various points of view from different smartglasses wearers 104, a history or log of the various actions performed by the smartglasses wearer 104 and/or the remote user 116 during the procedure, or other electronic resources.

Requests for these electronic resources may be made by the smartglasses wearer 104 and/or the remote user 116. Upon receipt of a request, the resources application 202 retrieves the requested electronic content and sends the content to the smartglasses 106 for displaying the electronic image as a virtual object. In addition, or in the alternative, the resources application 202 may send the electronic content to the remote client computing device 118 for viewing by the remote user 116 as an electronic file that can be accessed or downloaded through a web browser application.

The resources application 202 may be accessed to retrieve any electronic content not stored locally on the smartglasses computing device 119 or the remote client computing device 118. However, it should be appreciated that some electronic content may be stored locally on the smartglasses computing device 119 or the remote client computing device 118 for quick access. For instance, certain electronic content may be downloaded by the smartglasses computing device 119 or the remote client computing device 118 upon receipt from the resources application 202.

Various exemplary modules of the resources application 202 will now be described. In one example, the resources application 202 may include an EHR module 279 configured to receive and process a request for access to electronic patient information and records. In that regard, when a request is made, the EHR module 279 may retrieve the electronic information or record and send data containing the electronic information or record to the smartglasses 106 for display as a virtual object and/or to the remote client computing device 118 for viewing by the remote user 116. For instance, a snapshot of the patient history may be displayed as a virtual object 3286, as shown in the screen shot 2900 of the exemplary smartglasses GUI display of FIG. 35.

As another example, the resources application 202 may include an image module 281 configured to receive and process requests for an electronic image related to the patient. The image model 281 may receive a request for an image of the patient, and the module 281 may retrieve that image and send information to the smartglasses for displaying the electronic image as a virtual object and/or to the remote client computing device 118 for viewing by the remote user 116. For instance, FIG. 13 illustrates a screen shot 1300 of an exemplary remote client computing device GUI display showing an X-ray displayed as a virtual object 1344 for a smartglasses wearer 104. FIG. 36 illustrates a screen shot 3300 of an exemplary smartglasses GUI display showing an X-ray and CT scan displayed as a virtual object 3392.

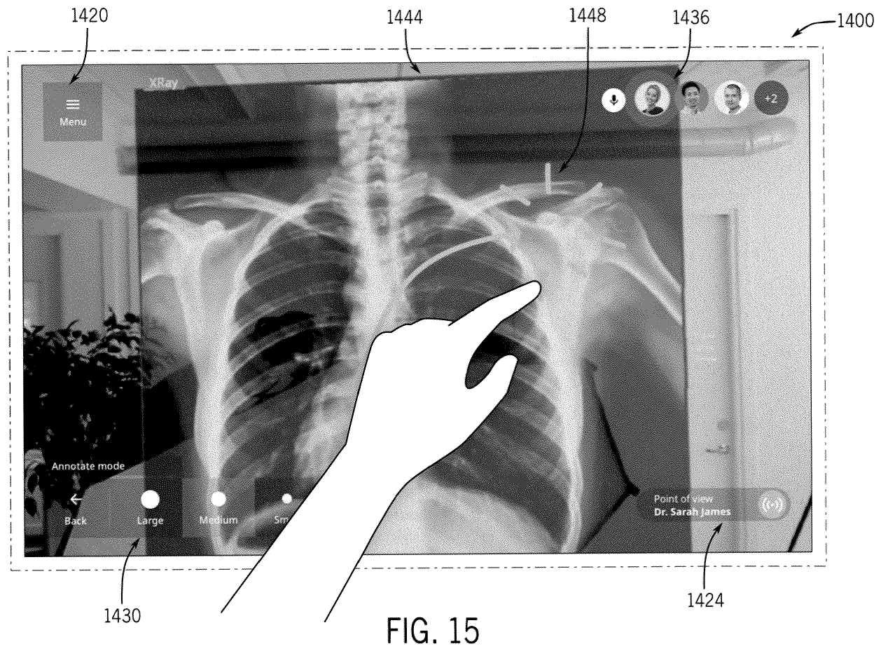

FIG. 15 illustrates a screen shot 1400 of an exemplary remote client computing device GUI display showing an X-ray scan displayed as a 2-D transparent image 1444. The annotation application 196 may be used to add an annotation 1448 to the 2-D transparent image 1444 shown in FIG. 15. Moreover, FIG. 16 illustrates a screen shot 1500 of an exemplary remote client computing device GUI display showing an X-ray scan displayed as a 2-D transparent image 1544 with an added annotation 1548, and further showing an option to send the annotated image to, for instance, the smartglasses wearer 104 by clicking a send button 1526.

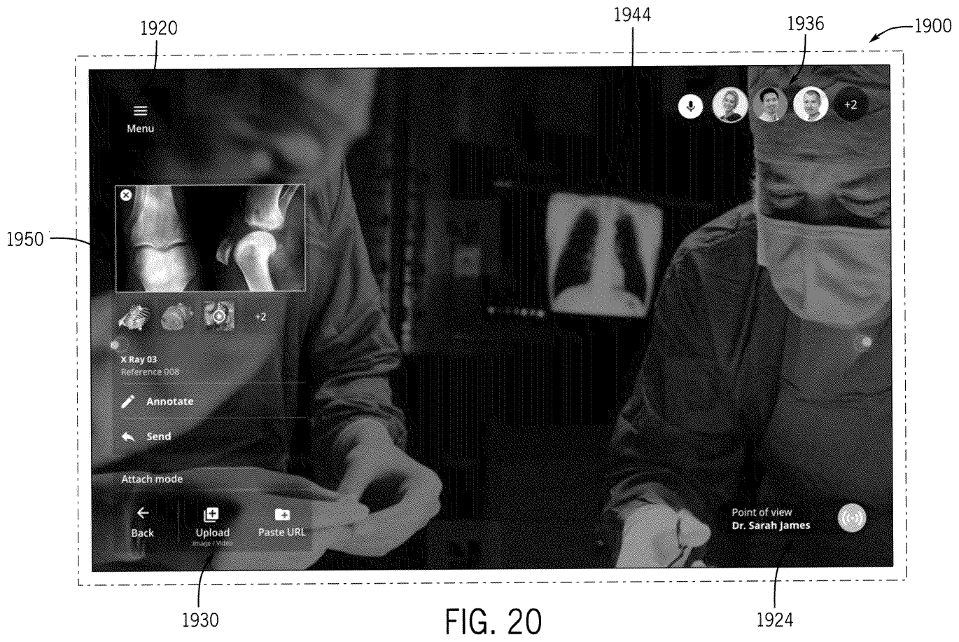

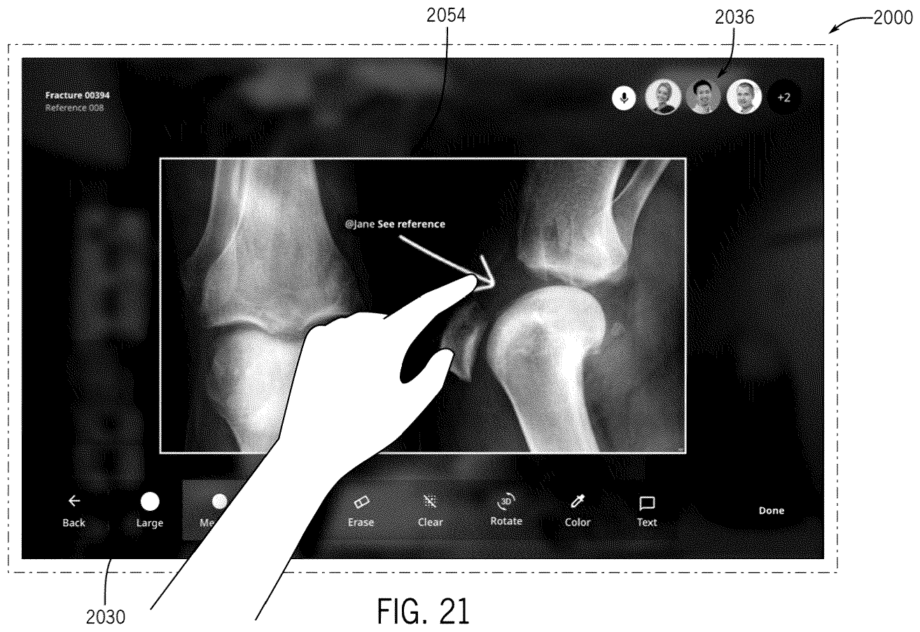

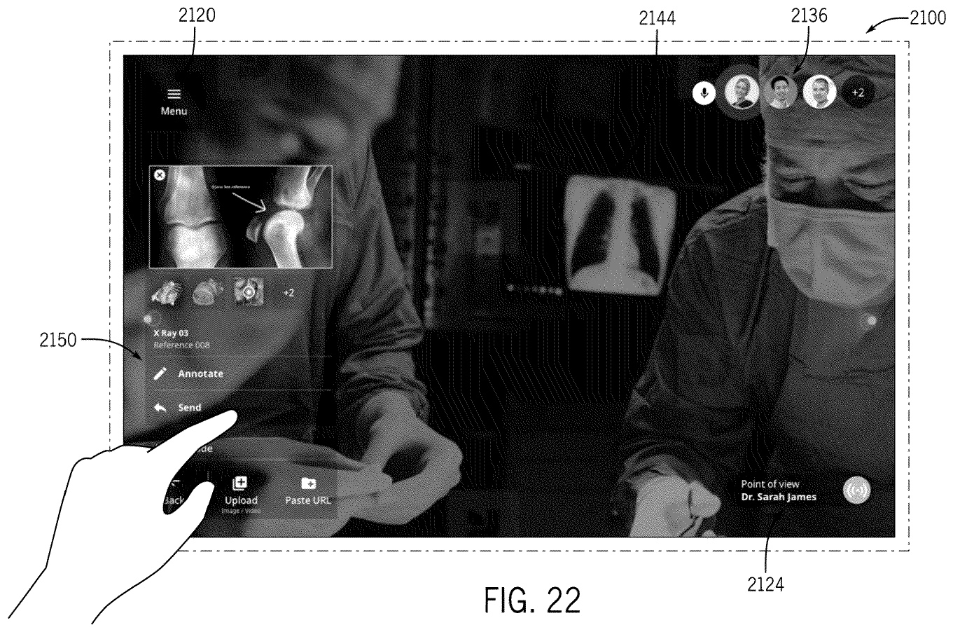

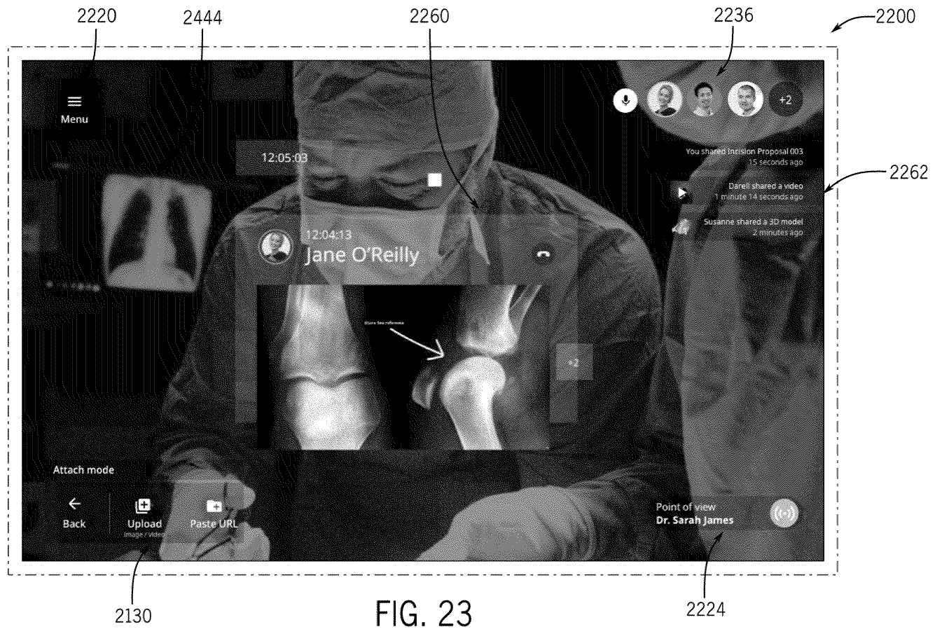

FIG. 20 illustrates a screen shot 1900 of an exemplary remote client computing device GUI display showing a resource menu displayed as a 2-D transparent image 1950. The resource menu 1950 may be displayed, for instance, after the remote user 116 selects an option to upload from a top level resource menu 1930. The remote user 116 can select an option to annotate an X-ray 2-D transparent image 2054, as shown in the screen shot 2000 of an exemplary remote client computing device GUI display of FIG. 21. The annotation application 196 may be used to add an annotation (not labeled) to the 2-D transparent X-ray image 2054 shown in FIG. 21. Moreover, FIG. 22 illustrates a screen shot 2100 of an exemplary remote client computing device GUI display showing an option to send the annotated image to, for instance, the smartglasses wearer 104 by clicking a “send” button in the resource menu displayed as a 2-D transparent image 2150. Finally, FIG. 23 illustrates a screen shot 2200 of an exemplary remote client computing device GUI display showing the annotated X-ray image received and displayed as a virtual image 2260 for viewing by the smartglasses wearer 104.

As yet another example, the resources application 202 may include an anatomy model module 283 configured to receive and process requests for displaying a 3-D anatomy model. For instance, the anatomy model module 283 may provide a menu displayed as a virtual object on the smartglasses 106 that allows the smartglasses wearer 104 to select a type or area of a 3-D anatomy model to be displayed. The anatomy model module 283 may similarly provide a menu option in the Web browser application of the remote client computing device 118 that allows the remote user 116 to select a type or area of a 3-D anatomy model to be displayed. Upon receiving a selected menu item, the anatomy model module 283 can display some or all portions of the 3-D anatomy model requested.

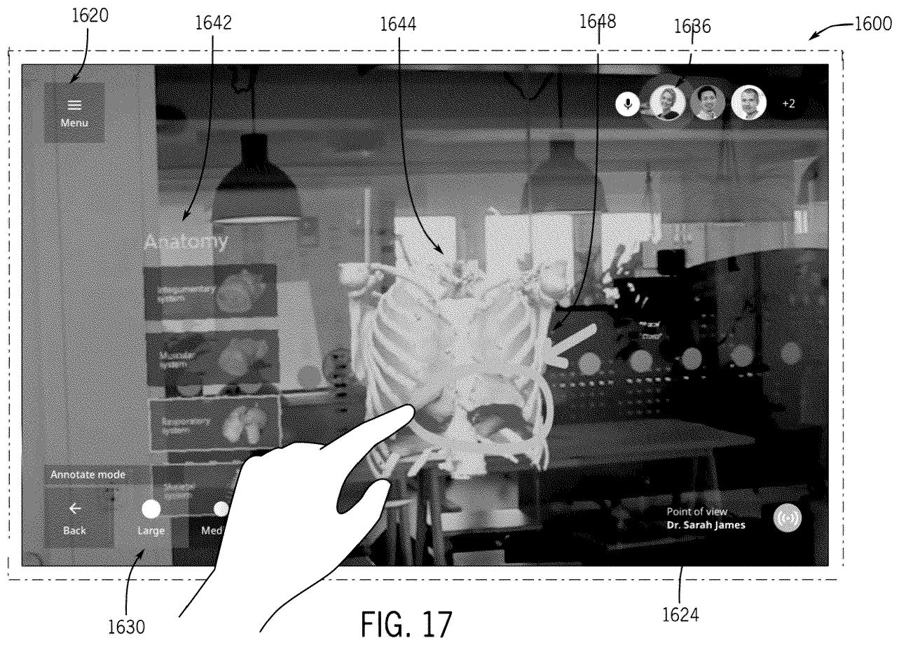

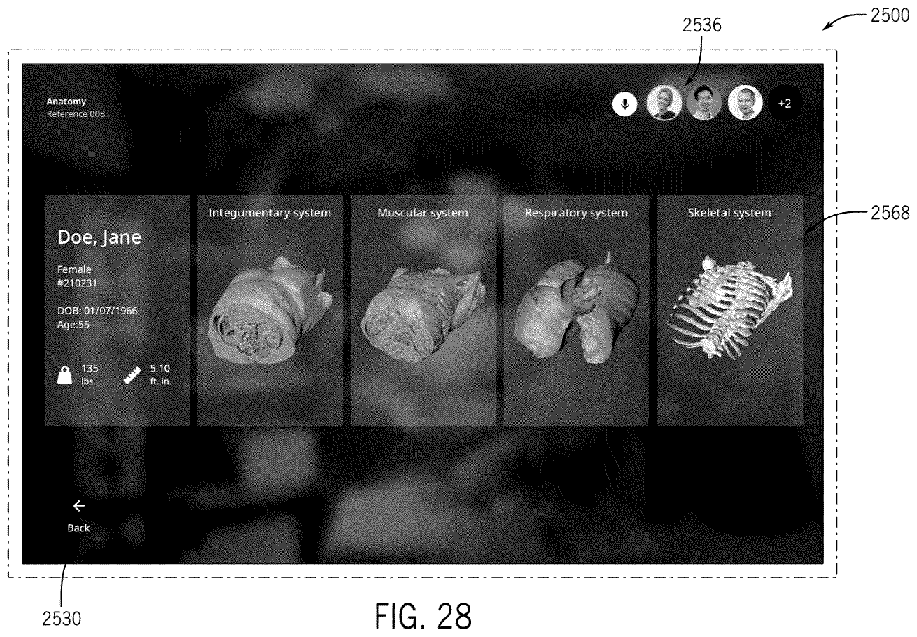

For instance, FIG. 17 illustrates a screen shot 1600 of an exemplary remote client computing device GUI display showing an anatomy menu and a 3-D anatomy model displayed as images 1642 and 1644, respectively. Of note, the annotation application 196 may be used to add an annotation 1648 to the 3-D anatomy model 1644 shown in FIG. 17. FIG. 28 also illustrates a screen shot 2500 of an exemplary remote client computing device GUI display showing an anatomy menu displayed as an image 2568.

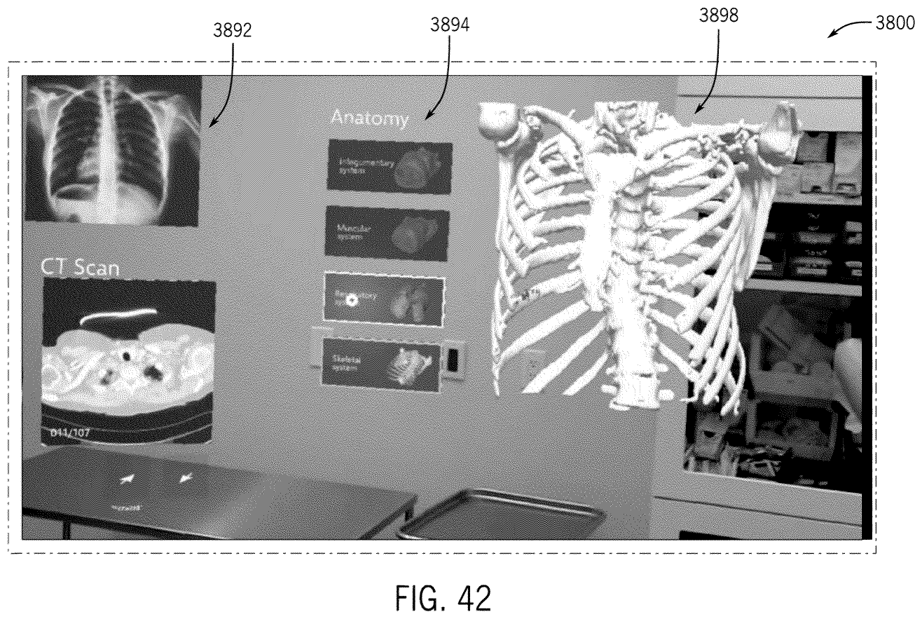

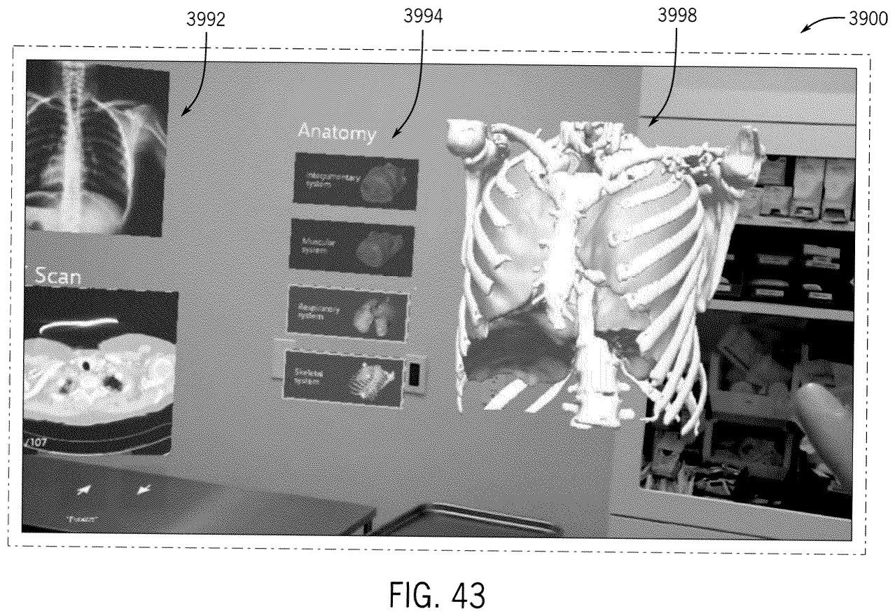

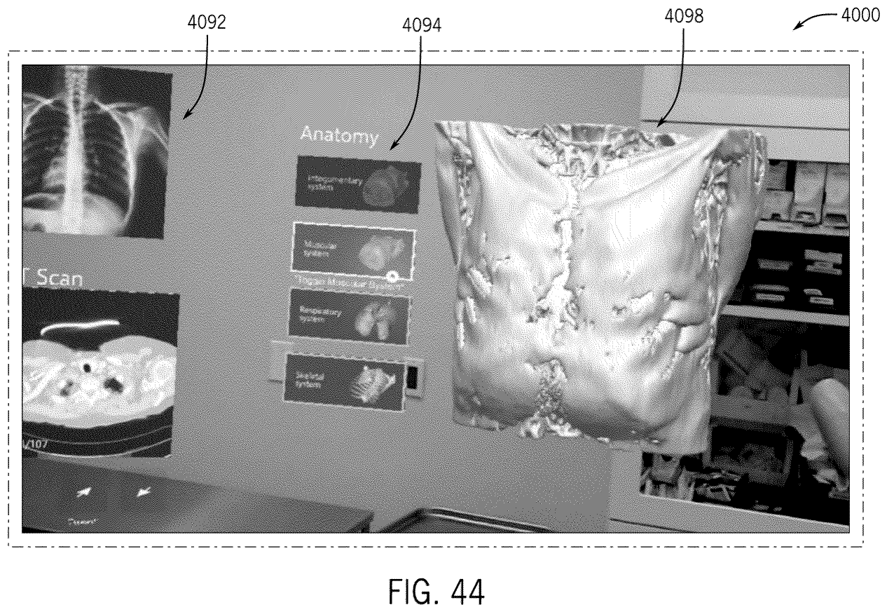

FIG. 36 illustrates a screen shot 3300 of an exemplary smartglasses GUI display showing an anatomy menu displayed as a virtual object 3394, and FIG. 42 illustrates a screen shot 3800 of an exemplary smartglasses GUI display showing a 3-D anatomy model displayed as a virtual object 3898. FIG. 43 illustrates a screen shot 3900 of an exemplary smartglasses GUI display showing a 3-D anatomy model displayed as a virtual object 3998, but with certain portions of the 3-D anatomy model turned on. For instance, the lungs in the 3-D anatomy model of FIG. 43 are turned on and the lungs in the 3-D anatomy model of FIG. 42 are turned off. Similarly, the muscular layer in the 3-D anatomy model of FIG. 44 is turned on and the muscular layer in the 3-D anatomy model of FIGS. 42 and 43 are turned off. The smartglasses wearer 104 or remote user 116 can request that certain portions of the model are turned on or off, and the anatomy model module 283 may receive and process those requests for displaying certain portions of the 3-D anatomy model.

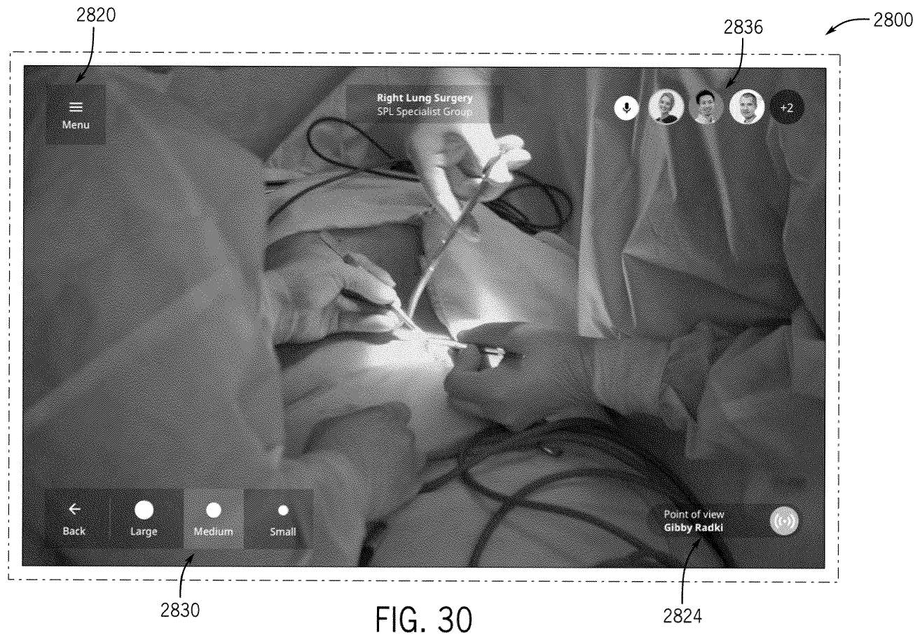

In another example, the resources application 202 may include a point of view module 285 configured to receive and process requests for changing the point of view of the smartglasses wearer in the session. For instance, a remote user may choose to change the point of view from a lead surgeon to a medical assistant, both wearing smartglasses, to see a different perspective of the procedure. For instance, FIG. 29A illustrates a screen shot 2600 of an exemplary remote client computing device GUI display showing a first point of view from a first smartglasses wearer, FIG. 29B illustrates a screen shot 2700 of an exemplary remote client computing device GUI display showing a menu 2724 for selecting a different point of view, and FIG. 30 illustrates a screen shot 2800 of an exemplary remote client computing device GUI display showing a second point of view from a second smartglasses wearer.

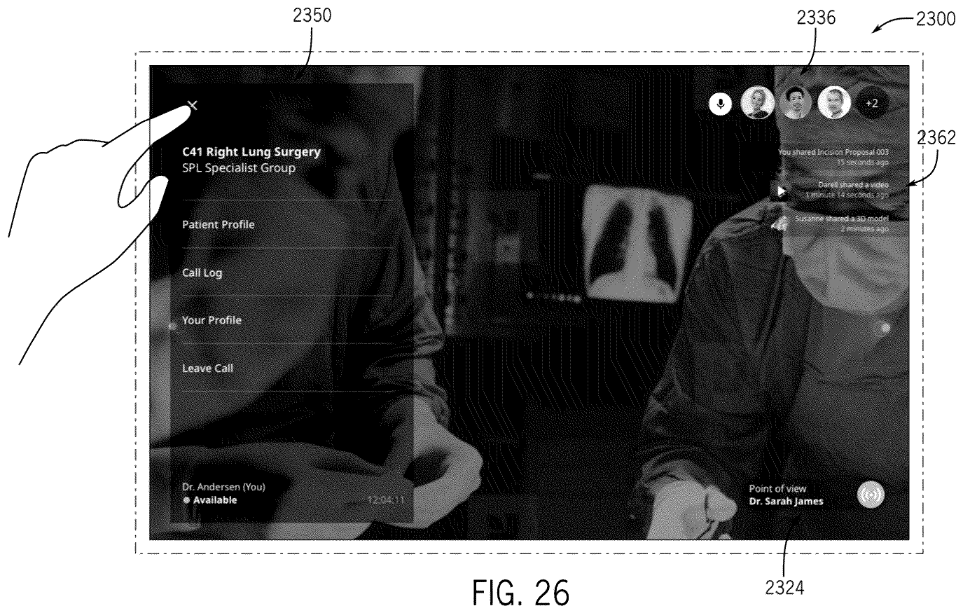

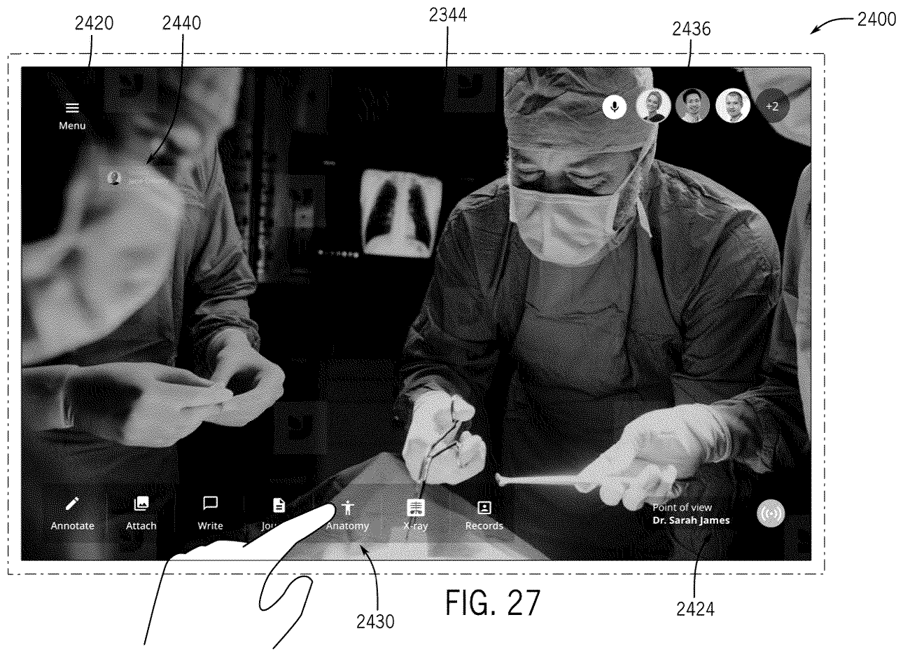

As yet another example, the resources application 202 may include an activity log module 287 configured to display a history of any activity or electronic communications between the wearable system and the remote client computing device, such as annotations, notes, sent files, etc., that have taken place during the communication session. For instance, FIG. 26 illustrates a screen shot 2300 of an exemplary remote client computing device GUI display showing an activity log displayed as an image 2632.

The resources application 202 may be configured to support access to any other type of electronic resource needed to support the procedure or task being performed by the smartglasses wearer that can be displayed as a virtual object. Moreover, the resources application 202 may be configured to support access to any other type of electronic resource needed to support the remote user 116 during the communication session.

Referring to FIG. 5G, the measurement application 204 of the communication management and information delivery platform 184 will now be described. The measurement application 204 is generally configured to communicate with the depth estimation application 198 and/or the zoom application 200 as needed to acquire depth and size data of any objects for accurately producing a measurement of an object.

The depth estimation sensor(s) 142 and/or the zoom sensors 144 may output one or more signals, which may be processed by an AR output module 280 for displaying a virtual measurement image on the display screen(s) of the smartglasses 106 and/or the remote client computing device 118. In addition or in the alternative, the depth estimation sensor(s) 142 and/or the zoom sensors 144 may be processed by an EHR output module 282 for sending measurement data to the healthcare facility computing system 124 for storage in an EHR of the patient or other secure databases. In addition or in the alternative, the depth estimation sensor(s) 142 and/or the zoom sensors 144 may be processed by a data analysis module 284 for use by cloud-based AI platforms for identifying or analyzing objects viewed by the smartglasses user (such as for anomaly detection).

Referring to FIG. 5H, the AR movement application 208 of the communication management and information delivery platform 184 will now be described. The AR movement application 208 is generally configured to allow the smartglasses wearer 104 to move a virtual object relative to an axis. Typically, spatial movements in an AR scene require placing the virtual object in a box and then moving the corners/lines of the box to move the object. With the AR movement application 208, on the other hand, the virtual object is moved without a box. For instance, a virtual object rotation module 288 may be used to rotate a virtual object about an axis, and a virtual object zoom module 292 may be used to increase or decrease the size of the virtual object relative to the axis.

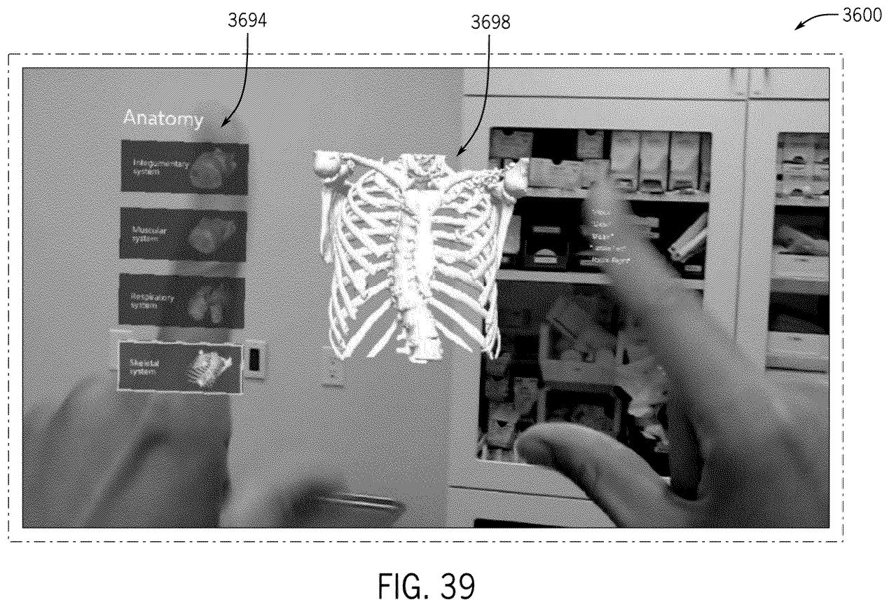

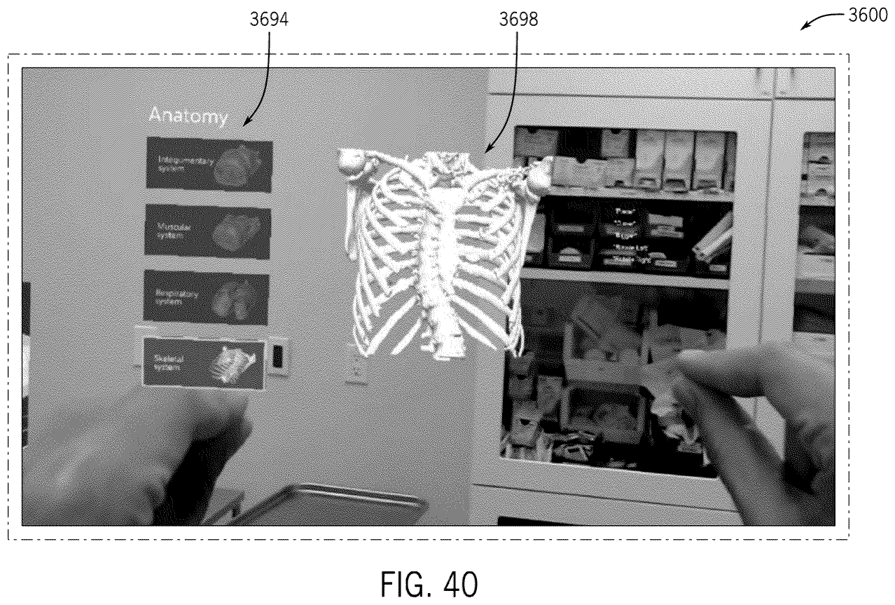

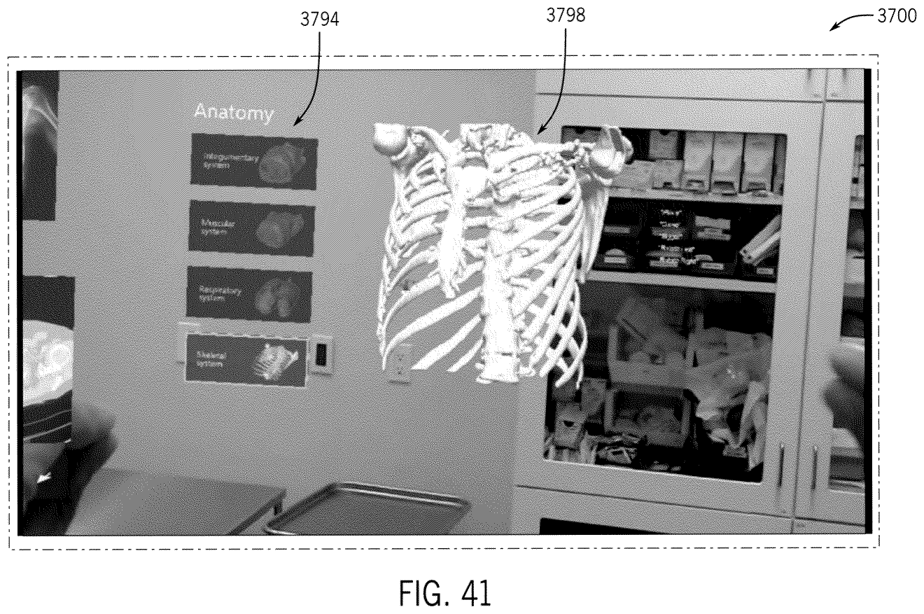

As a non-limiting example, FIGS. 39 and 40 illustrate a screen shot 3600 of an exemplary GUI display of the smartglasses 106 showing a 3-D anatomy model represented as a virtual image 3688. The smartglasses wearer 104 may initiate movement of the 3-D anatomy model with a first gesture of his/her hands and/or fingers (e.g., pressing the fingers together), as shown in FIGS. 39 and 40. After movement is initiated, the smartglasses wearer 104 may rotate the 3-D anatomy model (represented as virtual image 3798) about a vertical axis, as shown in the screen shot 3700 of FIG. 41. In a further aspect, the smartglasses wearer 104 may rotate the 3-D anatomy model (represented as virtual image 3898) relative to the vertical axis, as shown in the screen shot 3800 of FIG. 42.

Referring to FIG. 5I, the IoT application 210 of the communication management and information delivery platform 184 will now be described. The IoT application 210 is generally configured to associate virtual objects with IoT devices. These associations may enable a smartglasses wearer 104 to direct a control input to a particular IoT device and receive outputs such as sensor measurements or state information. For instance, the IoT devices may include medical equipment (e.g., an ultrasound, fluoroscopy, blood pressure monitor, heart rate monitor, etc.), building devices, etc. As an example, FIG. 35 illustrates a screen shot 3200 of an exemplary GUI display of the smartglasses 106 showing a laparoscope video feed image represented as a virtual image 3290. The smartglasses wearer 104 may access and control the laparoscope as an IoT device.

In that regard, the IoT application 210 may include an IoT device library module 294 that stores information for each IoT device associated with the smartglasses 106, an IoT AR module 296 that is configured to represent output aspects of the IoT device as a virtual object in the smartglasses display, and an IoT function module 298 that is configured to control aspects of the IoT device through inputs of the smartglasses wearer 104.

Referring to FIG. 5J, the data integration application 212 of the communication management and information delivery platform 184 will now be described. The data integration application 212 is generally configured to receive, process, and package data for securely sending the data between devices on the network 120. For instance, the data integration application 212 may include an EHR module 302 configured to receive, process, and package data for securely sending the data between the smartglasses 106 and/or the remote client computing device 118 and the healthcare facility computing system 124. As an example, FIG. 13 illustrates a screen shot 1300 of an exemplary GUI display of the remote client computing device 118 showing a menu represented as an image 1330. The remote user 116 may select an option from the menu 1330 (e.g., annotate, attach, write, journal, or records) to send data to the healthcare facility computing system 124 or another device on the network 120.

As another example, the EHR module 302 may receive audio data from the smartglasses 106 and/or the smartglasses computing device 119 (through, for instance, the audio transcription module 236) and package and securely send the audio-to-text data to the healthcare facility computing system 124 for updating an electronic health record (EHR) of the patient. FIG. 35 illustrates a screen shot 3200 of an exemplary GUI display of the smartglasses 106 showing a dictation button represented as a virtual image 3288. The smartglasses wearer 104 may select the dictation button to send for instance, an audio-to-text data to the healthcare facility computing system 124 for updating an electronic health record (EHR) of the patient.

The EHR module 302 may also receive text or image data from the healthcare facility computing system 124 and package and send that data to the smartglasses 106 and/or the smartglasses 106 and/or the remote client computing device 118 for viewing on the display of that user. For instance, a snapshot of the patient history may be displayed as a virtual object 3286, as shown in the screen shot 2900 of the exemplary smartglasses GUI display of FIG. 35.

The data integration application 212 may further include a medical device module 306 configured to receive, process, and package data for securely sending the data between the smartglasses 106 and/or the remote client computing device 118 and external medical devices, such as a fluoroscopy, an ultrasound, etc. For instance, image data captured on a laparoscope device may be sent to the smartglasses 106 and/or the remote client computing device 118 for viewing on the display of that user. FIG. 35 illustrates a screen shot 3200 of an exemplary GUI display of the smartglasses 106 showing a laparoscope video feed image represented as a virtual image 3290. The medical device module 306 may also receive audio data from smartglasses 106 and/or the remote client computing device 118 (through, for instance, the audio transcription module 236) and package and send the audio-to-text data to the external medical device for controlling the device.

The data integration application 212 may further include an external services module 310 configured to receive, process, and package data for securely sending the data between the smartglasses 106 and/or the remote client computing device 118 and external services (e.g. cloud-based AI platforms). For instance, an image captured by the medical multimedia data capture module 138 may be received, processed, and repackaged by the external services module 310 for sending to a remote service for analysis. As a specific example, an image of an organ, tissue, etc., may be analyzed by an ML algorithm for tumor detection. The external services module 310 may be used to process any data received by the smartglasses 106 and/or the remote client computing device 118 for downstream analysis.

Referring to FIG. 5K, the device management application 216 of the communication management and information delivery platform 184 will now be described. In general, the device management application 216 is configured to secure the protected health information (PHI) or personally identifiable information (PII) maintained on the network devices. For instance, the software and infrastructure supporting the communication management and information delivery system 100 may conform to industry best practices as outlined in the NIST Cybersecurity Framework, including guidance from international standards NIST 800-53 R4, Center for Internet Security, ISO27001, COBIT 5, as well as Google and AWS cloud service providers.

In one aspect, the device management application 216 includes a secure data/communication module 314 configured to exchange encrypted and anonymized information between the network devices. In that regard, the secure data/communication module 314 may be configured to preserve information security, privacy, confidentiality, integrity, and availability of data sent over the network 120. The secure data/communication module 314 may further be configured to safeguard all data stored on network devices, including any remote computing devices which are not centrally managed. In that regard, the secure data/communication module 314 ensures HIPAA Compliance for the communication management and information delivery system 100.

In one aspect, application containers may be scanned and monitored for vulnerabilities via Clair, which is an open source project for the static analysis of vulnerabilities in application containers (currently including OCI and docker). Clair API can be used to index container images and can then match it against known vulnerabilities.

Referring back to FIG. 4, the computing device 125, which may be used to allow an authorized person to interact with the application computing system 122, will now be described. The computing device 125 may be any suitable device having a processor 190 and memory 194 (including software/firmware code (SW) 195), and an input/output (I/O) controller 206. The input/output (I/O) controller 178 may be configured to connect the computing device 125 to one or more devices, such as a keyboard/mouse, etc. All of the components of the computing device 125 may communicate, directly or indirectly, with one another via one or more buses 218.

A user interface module 214 of the computing device 125 may enable a person to interact with the application computing system 122. For example, the user interface module 214 may include a visual display such as a display screen, an audio device such as a speaker, and various input devices such as a keyboard, touch-screen, microphone, or the like. Multimodal inputs and outputs may be provided as well. In some embodiments, the user interface module 214 may communicate with a remote or external device through the I/O controller 206.

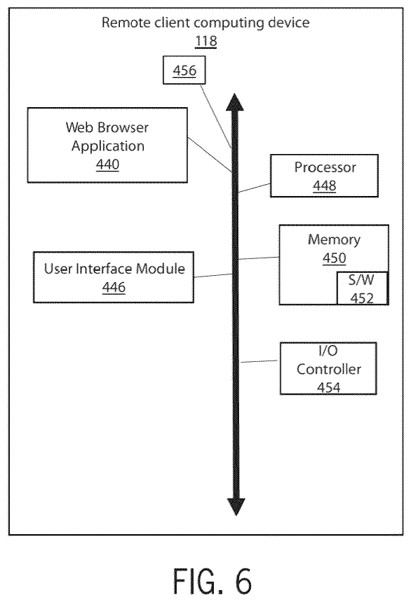

Referring to FIG. 6, the remote client computing device 118, which may be used to allow an authorized person to interact with the smartglasses 106 or other network devices, will now be described. The remote client computing device 118 may be any suitable device having a processor 448 and memory 450 (including software/firmware code (SW) 452), and an input/output (I/O) controller 454. The input/output (I/O) controller 454 may be configured to connect the remote client computing device 118 to one or more devices, such as a keyboard/mouse, etc. All of the components of the remote client computing device 118 may communicate, directly or indirectly, with one another via one or more buses 456.

A user interface module 446 may enable a person to interact with the remote client computing device 118. For example, the user interface module 446 may include a visual display such as a display screen, an audio device such as a speaker, and various input devices such as a keyboard, touch-screen, microphone, or the like. Multimodal inputs and outputs may be provided as well. In some embodiments, the user interface module 446 may communicate with a remote or external device through the I/O controller 454.

The remote client computing device 118 may include a web browser application 440 (accessible through a Website) configured to facilitate interaction of the remote client computing device 118 with the smartglasses 106 and other network devices. Remote users, such as surgeons, doctors, medical staff members, nurses, assistant doctors, medical students, patients, caretakers of the patients and the like may access a Website over the network 120 using a web browser application 440 installed in their respective electronic devices and thereafter use the services of the application.

The web browser application 440 enables the remote user 116 to register/login and accept incoming calls to join an interactive communication session with the smartglasses 106. FIGS. 10-12 show screen shots of exemplary web browser application GUI displays of the remote client computing device 118 for registering/logging in and accept incoming calls.

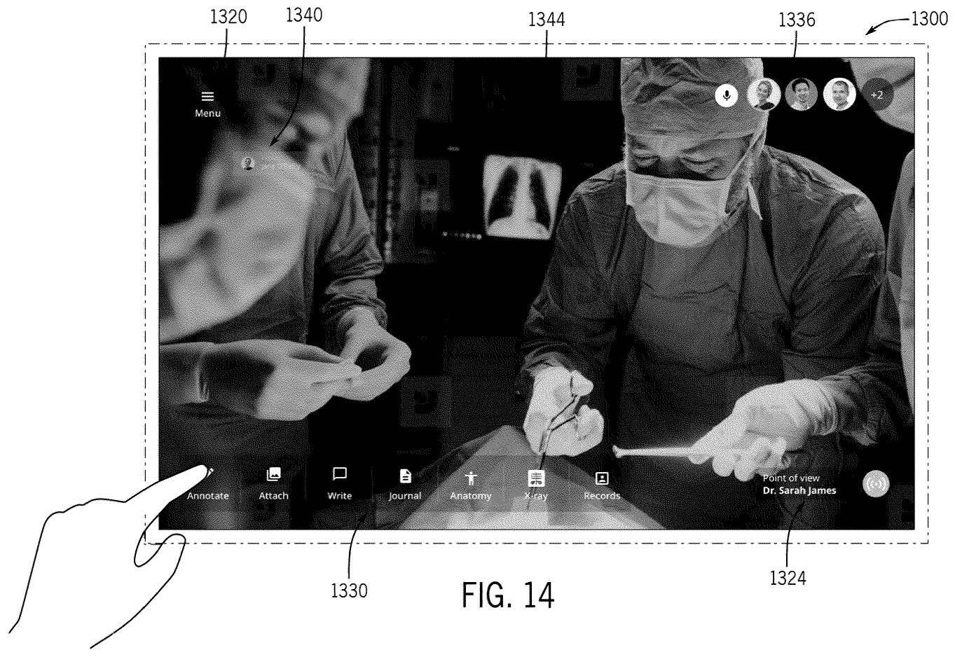

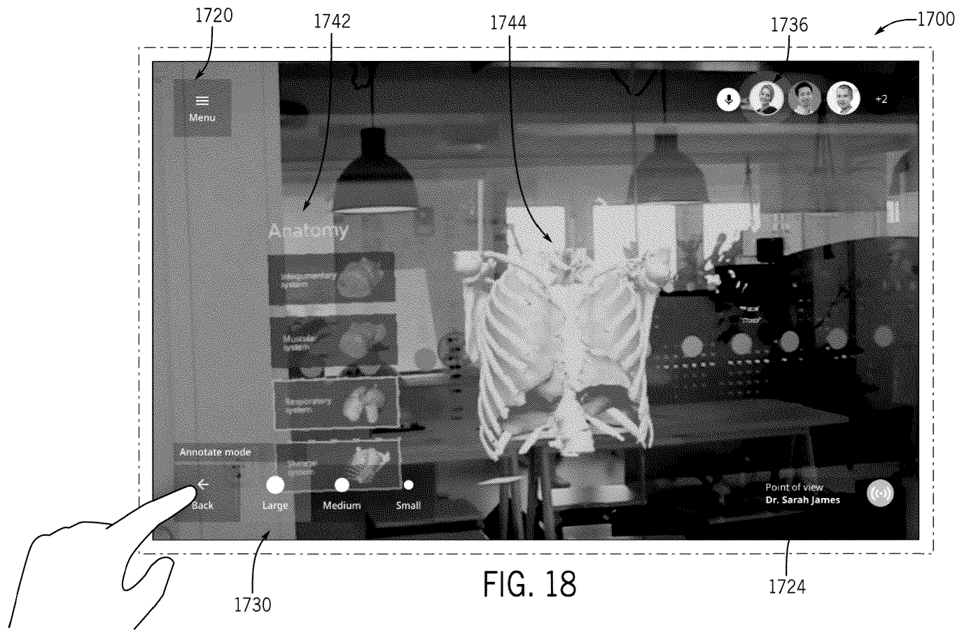

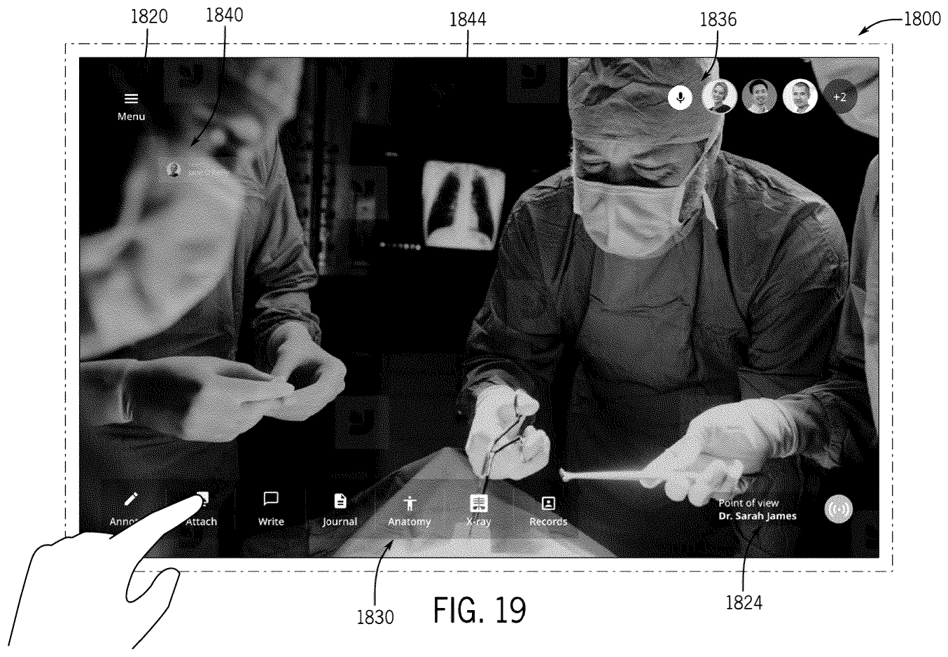

The web browser application 440 also interacts with the communication application 188 of the application computing system 122 to facilitate audio, video, and/or text communications between the smartglasses 106 and the remote client computing device 118. Regarding the video communications between the smartglasses 106 and the remote client computing device 118, FIGS. 13-31 show screen shots of exemplary GUI displays of the remote client computing device 118 with a live video feed of the smartglasses wearer 104 as a background for the web browser application 440.

The web browser application 440 may further interact with the resources application 202 of the application computing system 122 to enable the remote user 116 to provide annotations, add text notes, select a smartglasses wearer 104 point of view, access images/files, annotate and send images/files to the smartglasses wearer 104, etc. For instance, FIG. 13 illustrates a screen shot 1300 of an exemplary GUI display of the remote client computing device 118 showing a menu represented as an image 1330. The remote user 116 may select an option from the menu 1330 (e.g., annotate, attach, write, journal, anatomy, X-ray, or records) to access a resource and/or perform one or more actions with those resources.

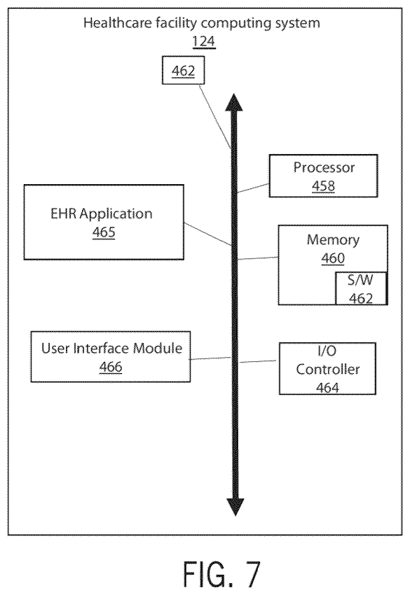

Referring to FIG. 7, the healthcare facility computing system 124, which may be configured to facilitate a private network for the healthcare facility 102, will now be described. The healthcare facility computing system 124 may be any suitable device having a processor 458 and memory 460 (including software/firmware code (SW) 462), and an input/output (I/O) controller 464. The I/O controller 464 may be configured to connect the healthcare facility computing system 124 to one or more devices, such as a keyboard/mouse, etc. All of the components of the healthcare facility computing system 124 may communicate, directly or indirectly, with one another via one or more buses 462.

A user interface module 466 may enable a person to interact with the healthcare facility computing system 124. For example, the user interface module 466 may include a visual display such as a display screen, an audio device such as a speaker, and various input devices such as a keyboard, touch-screen, microphone, or the like. Multimodal inputs and outputs may be provided as well. In some embodiments, the user interface module 466 may communicate with a remote or external device through the I/O controller 464.

The healthcare facility computing system 124 includes an EHR application 465 configured to securely receive, process, and store data related to a patient's record. The EHR application 465 may interact with the EHR module 279 of the resources application 202 of the application computing system 122 to enable access to authorized EHR records by other network devices. The EHR application 465 may also interact with the EHR output module 282 of the measurement application 204 of the application computing system 122 to receive measurement data from a network device. Moreover, the EHR application 465 may interact with the EHR module 302 of the data integration application 212 of the application computing system 122 to securely receive and process data related to a patient's record sent from a network device.

It should be appreciated that the components of each computing device/system shown in FIGS. 3-7 are exemplary only, and fewer or more than the components shown may be used. Moreover, the components of each computing device/system may instead be included in other computing devices/systems than that shown. Further, it should be noted that at least some of the components described above in connection with the environment may be optional and thus in some example embodiments may include more, less or different components than those described or shown.

### Exemplary Interactions of the Components of the Communication Management and Information Delivery System

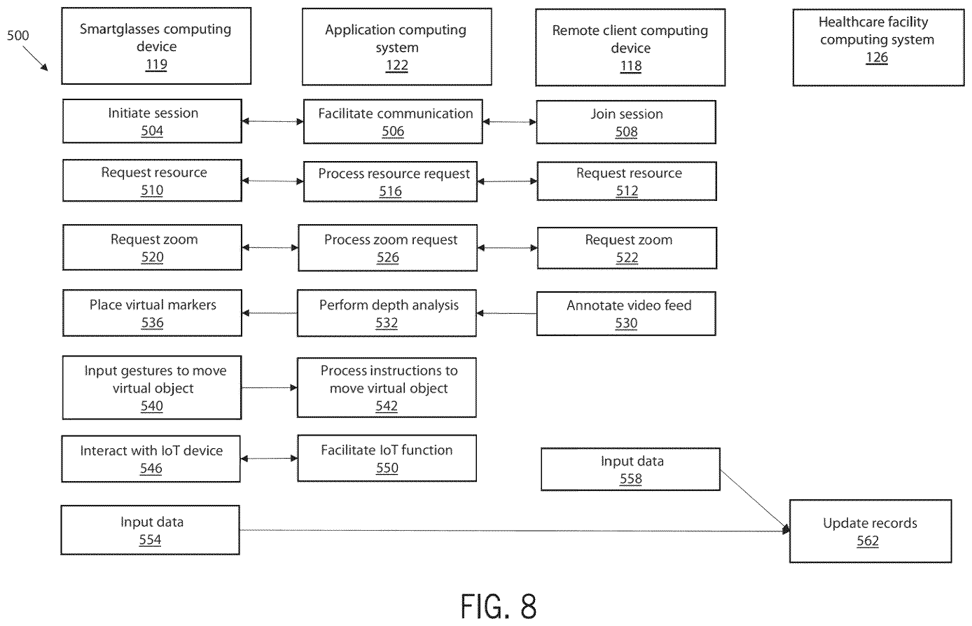

FIG. 8 shows a swim diagram illustrating some of the various ways components of the communication management and information delivery system 100 can interact. It should be appreciated that not all the component interactions are shown, and the actions performed by one component may instead be carried out by one or more of the other components. Moreover, although not described in detail in this section, the component interactions may be carried out using one or more of the applications described above.

In the example shown, the smartglasses computing device 119 may initiate a communication session with the remote client computing device 118 at block 504. The application computing system 122 can facilitate the communication at block 506, such that the remote client computing device 118 can join the communication session with the smartglasses 106 at block 508.

At blocks 510 and 512, the smartglasses wearer 104 and the remote user 116 may request a resource (through the smartglasses computing device 119 and the remote client computing device 118, respectively), such as patient records or data, electronic images (such as MRI, X-ray, or CAT scan images), 3-D anatomy models, various points of view from different smartglasses wearers 104, a history or log of the various actions performed by the smartglasses wearer 104 and/or the remote user 116 during the procedure, or other resources. The application computing system 122 can process the request(s) at block 516 for sending the requested resources to the smartglasses computing device 119 or the remote client computing device 118.

At blocks 520 and 522, the smartglasses wearer 104 and the remote user 116 may request a zoomed-in image (through the smartglasses computing device 119 and the remote client computing device 118, respectively), such as a close-up view of a real life object being viewed by the smartglasses wearer. The application computing system 122 can process the request(s) at block 526 to produce a zoomed-in image data file for viewing on the display screen of the smartglasses 106 or the remote client computing device 118.

The smartglasses wearer 104 may initiate movement of a virtual object through one or more gestures. At block 540, the gestures are received as input by the smartglasses computing device 119 and sent to the application computing system 122 for processing. At block 542, the application computing system 122 processes the input from the smartglasses computing device 119 and outputs data for moving the virtual object on the display of the smartglasses 106.

At block 546, the smartglasses computing device 119 may interact with an IoT device (not shown) by sending data to and receiving data from the application computing system 122. The application computing system 122 sends data to and receives data from the IoT device at block 550.

At blocks 554 and 556, the smartglasses computing device 119 and/or the remote client computing device 118 may be used to input data, such as dictation, notes, etc., and that inputted data may be uploaded to the healthcare facility computing system 124 at block 562. For instance, audio files may be converted to text for updating an HER of the patient.

### Exemplary Method for Initiating a Secure Communication Session

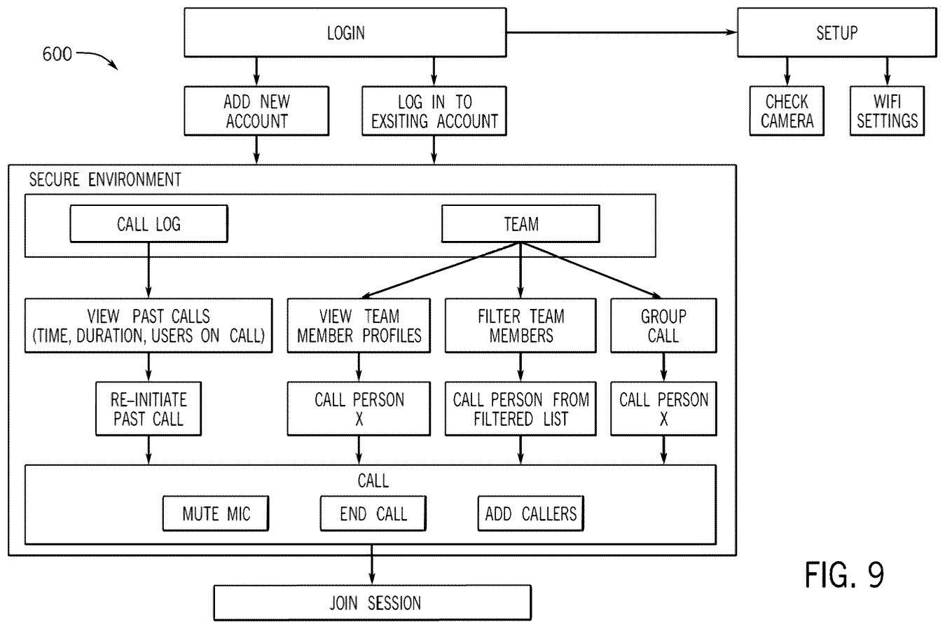

FIG. 9 illustrates an example method 600 for initiating a secure communication session between the smartglasses 106 and the remote client computing device 118, which may be carried out at least in part by the communication application 188 and/or the user authentication application 192. The method may be initiated by the smartglasses wearer 104 or the remote user 116.

FIGS. 10A-10C, 11, and 12, discussed in detail above, show screen shots of exemplary GUI displays of the remote client computing device 118 that may be used to carry out aspects of the example method 600. FIGS. 32 and 33A-33D, discussed in detail above, show screen shots of exemplary GUI displays of the smartglasses 106 that may be used to carry out aspects of the example method 600.

Although the example method 600 depicts a particular sequence of operations, the sequence may be altered without departing from the scope of the present disclosure. For example, some of the operations depicted may be performed in parallel or in a different sequence that does not materially affect the function of the method 600. In other examples, different components of an example device or system that implements the method 600 may perform functions at substantially the same time or in a specific sequence.

### Screen Shots of Exemplary GUI Displays

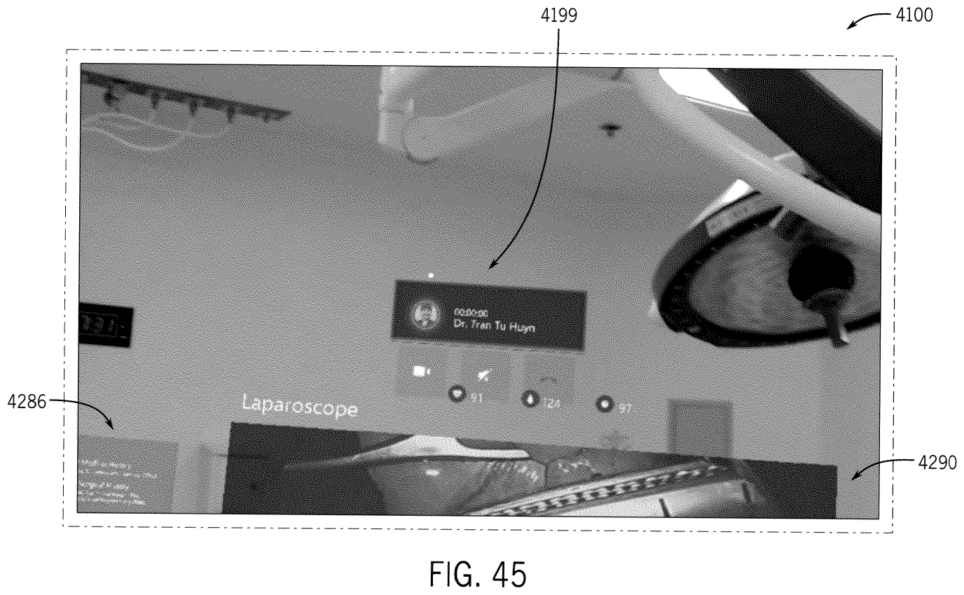

FIGS. 10-31 and A-U show screen shots of exemplary GUI displays of the remote client computing device 118 for use with the systems and methods described herein. FIGS. 32-45 and V-MM show screen shots of exemplary GUI displays of the smartglasses 106 for use with the systems and methods described herein.

For ease of reference and simplicity, similar images and virtual objects are labeled with similar reference numerals except in the '1000 series corresponding to the screen shot reference numeral used for that FIG.

A description of the screen shots of exemplary GUI displays of the remote client computing device 118 and the smartglasses 106 are described above with respect to the detailed description of the functionality of the smartglasses computing device 119, the application computing system 122, the remote client computing device 118, and the healthcare facility computing system 124. Accordingly, a description of the screen shots of exemplary GUI displays is not provided in this section for brevity.

For ease of reference and simplicity, similar images and virtual objects are labeled with similar reference numerals except in the '1000 series corresponding to the screen shot reference numeral used for that FIG.

It should be appreciated that various aspects of the exemplary GUI displays are ornamental in nature. In that regard, FIGS. 10-45 and A-MM are screen shots of exemplary GUI displays showing new designs.

FIGS. 10-31 show new designs of exemplary GUI displays of a computing device, wherein the GUI displays of FIGS. 13-31 and A-U show a live video feed as a background (such as live video feed of a real-world scene viewed by a smartglasses wearer). It should be appreciated that the live video feed is exemplary only and should be considered environment for the design.

FIGS. 32-45 and V-MM show screen shots of exemplary GUI displays of a wearable computing device (such as smartglasses 106), wherein the GUI displays of FIGS. 32-45 and V-MM show a real-world scene viewed by the wearer as background. It should be appreciated that the real-world scene is exemplary only and should be considered environment for the design.

The screen shots of exemplary GUI displays shown in FIGS. 10-45 and A-MM may be considered two-dimensional images shown on a computer screen, monitor, smartglasses display, or other display panel, or a portion thereof, such as the remote client computing device 118 or the smartglasses 106. In that regard, although not always shown, a broken or dashed outline of a computer screen, monitor, smartglasses display, or other display panel, or a portion thereof may be added to any of the drawings to represent an article of manufacture showing the ornamental design for the GUI display.

The designs include any and all parts, portions, elements, and/or combinations thereof of the exemplary GUI displays shown in the FIGS. The designs may also include any part, portion, element, and/or combination thereof of the disclosed designs, including a design that replaces any solid line with a broken line to disclaim any part, portion, element and/or combination thereof of the disclosed design, or to replace any broken line with a solid line to claim any part, portion, element and/or combination thereof of the disclosed designs.

Any thin solid lines that are now shown in the FIGS. or later added may represent contours only and will not necessarily illustrate an ornamentation of decoration on the surface of an article.

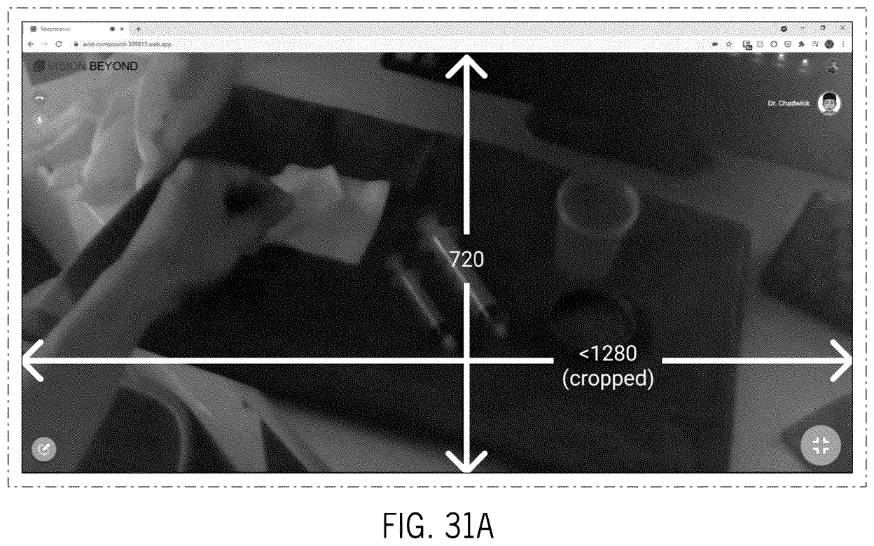

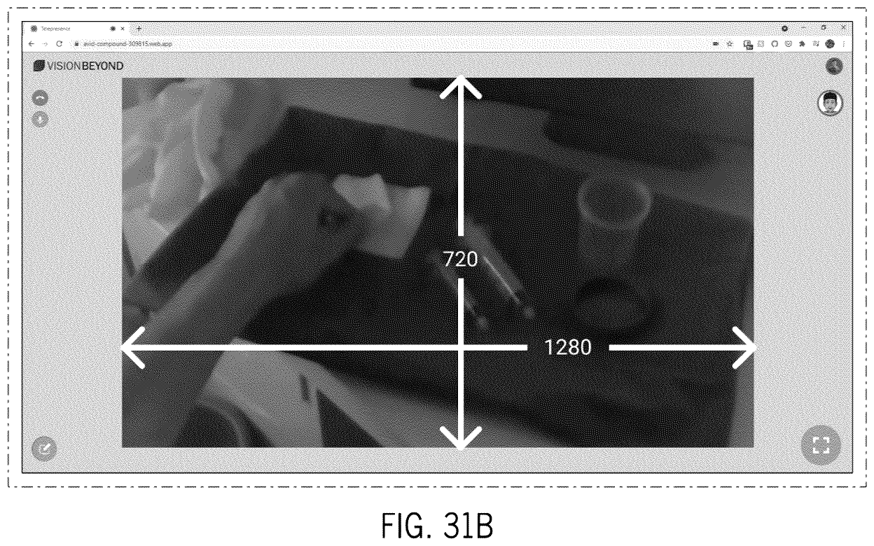

In some instances, the exemplary GUI displays showing new designs may be displayed on a computing device in a full screen mode, as shown in FIG. 31A. In the full screen mode, any computer icons or other GUI images are superimposed over a live video stream from an ongoing call between, for instance, the smartglasses wearer 104 and the remote user. Any of the exemplary GUI displays may instead be shown in a window screen mode, as shown in FIG. 31B. In the window screen mode, at least some of the computer icons or other GUI images may be in an area surrounding a live video stream from an ongoing call between, for instance, the smartglasses wearer 104 and the remote user. It should be appreciated that any of the exemplary GUI displays showing new designs may be displayed on a device in a full screen mode or in a window screen mode.

### AI Platforms

Aspects of the systems and methods described herein may use certain AI platforms to process data. For instance, computer vision may be used to run analyses of data over and over until it discerns distinctions and ultimately recognize images. For example, computer vision may be used to recognize/analyze a human organ, for instance, to determine if the organ has an anomaly. To train a computer to recognize a human organ and detect any anomalies, it needs to be fed vast quantities of organ images and related items to learn the differences and recognize an organ (with or without an anomaly).

Two technologies are commonly used to accomplish such image recognition: a type of machine learning called deep learning and a convolutional neural network (CNN).

Machine learning uses algorithmic models that enable a computer to teach itself about the context of visual data. If enough data is fed through the model, the computer will “look” at the data and teach itself to tell one image from another. Algorithms enable the machine to learn by itself, rather than someone programming it to recognize an image.

A CNN is an artificial neural network that can help a machine learning or deep learning model “look” by breaking images down into pixels that are given tags or labels. It uses the labels to perform convolutions (a mathematical operation on two functions to produce a third function) and makes predictions about what it is “seeing.” The neural network runs convolutions and checks the accuracy of its predictions in a series of iterations until the predictions start to come true. It is then recognizing or seeing images in a way like humans.

An artificial neural network attempts to replicate, using computer technology, logical reasoning performed by the biological neural networks that constitute animal brains. Deep neural networks, such as convolutional neural networks, are widely used for numerous applications, such as object detection, object classification, object tracking, big data analysis, among others. For example, convolutional neural networks can be used to extract high-level features, such as organ shapes, from an input image, and use these high-level features to output a probability that, for example, an input image includes a particular object.

FIG. 46 illustrates an example neural network architecture for use with the various computing devices and applications described herein. Architecture 4600 includes a neural network 4610 defined by an example neural network description 4601 in rendering engine model (neural controller) 4630. The neural network 4610 can represent a neural network implementation of a rendering engine for rendering media data. The neural network description 4602 can include a full specification of the neural network 4610, including the neural network architecture 4600. For example, the neural network description 4602 can include a description or specification of the architecture 4600 of the neural network 4610 (e.g., the layers, layer interconnections, number of nodes in each layer, etc.); an input and output description which indicates how the input and output are formed or processed; an indication of the activation functions in the neural network, the operations or filters in the neural network, etc.; neural network parameters such as weights, biases, etc.; and so forth.

The neural network 4610 reflects the architecture 4600 defined in the neural network description 4602. In this example, the neural network 4610 includes an input layer 4602, which includes input media data, such as object images, video feed frames, depth estimation sensor data, zoom sensor data, etc. In one illustrative example, the input layer 4602 can include data representing a portion of the input media data such as a patch of data or pixels (e.g., a 128×128 patch of data) in an image corresponding to the input media data.

The neural network 4610 includes hidden layers 4604A through 4604N (collectively “4604” hereinafter). The hidden layers 4604 can include n number of hidden layers, where n is an integer greater than or equal to one. The number of hidden layers can include as many layers as needed for a desired processing outcome and/or rendering intent. The neural network 4610 further includes an output layer 4606 that provides an output (e.g., a virtual image) resulting from the processing performed by the hidden layers 4604. In one illustrative example, the output layer 4606 can provide an identification of an object (e.g., a human organ) for identifying an anomaly. In another illustrative example, the output layer 4606 can provide an identification of an object depth for placing an annotation.

The neural network 4610 in this example is a multi-layer neural network of interconnected nodes. Each node can represent a piece of information. Information associated with the nodes is shared among the different layers and each layer retains information as information is processed. In some cases, the neural network 4610 can include a feed-forward neural network, in which case there are no feedback connections where outputs of the neural network are fed back into itself. In other cases, the neural network 4610 can include a recurrent neural network, which can have loops that allow information to be carried across nodes while reading in input.

Information can be exchanged between nodes through node-to-node interconnections between the various layers. Nodes of the input layer 4602 can activate a set of nodes in the first hidden layer 4604A. For example, as shown, each of the input nodes of the input layer 4602 is connected to each of the nodes of the first hidden layer 4604A. The nodes of the hidden layer 4604A can transform the information of each input node by applying activation functions to the information. The information derived from the transformation can then be passed to and can activate the nodes of the next hidden layer (e.g., 4604B), which can perform their own designated functions. Example functions include convolutional, up-sampling, data transformation, pooling, and/or any other suitable functions. The output of the hidden layer (e.g., 4604B) can then activate nodes of the next hidden layer (e.g., 4604N), and so on. The output of the last hidden layer can activate one or more nodes of the output layer 4606, at which point an output is provided. In some cases, while nodes (e.g., nodes 4608A, 4608B, 4608C) in the neural network 4610 are shown as having multiple output lines, a node has a single output and all lines shown as being output from a node represent the same output value.

In some cases, each node or interconnection between nodes can have a weight that is a set of parameters derived from training the neural network 4610. For example, an interconnection between nodes can represent a piece of information learned about the interconnected nodes. The interconnection can have a numeric weight that can be tuned (e.g., based on a training dataset), allowing the neural network 4610 to be adaptive to inputs and able to learn as more data is processed.

The neural network 4610 can be pre-trained to process the features from the data in the input layer 4602 using the different hidden layers 4604 in order to provide the output through the output layer 4606. In an example in which the neural network 4610 is used to provide an identification of an object depth for placing an annotation or identifying an anomaly, the neural network 4610 can be trained using training data that includes example images and object features of real world environments. For instance, training images can be input into the neural network 4610, which can be processed by the neural network 4610 to generate outputs which can be used to tune one or more aspects of the neural network 4610, such as weights, biases, etc.

In some cases, the neural network 4610 can adjust weights of nodes using a training process called backpropagation. Backpropagation can include a forward pass, a loss function, a backward pass, and a weight update. The forward pass, loss function, backward pass, and parameter update is performed for one training iteration. The process can be repeated for a certain number of iterations for each set of training media data until the weights of the layers are accurately tuned.

For a first training iteration for the neural network 4610, the output can include values that do not give preference to any particular class due to the weights being randomly selected at initialization. For example, if the output is a vector with probabilities that the object includes different features, the probability value for each of the different object features may be equal or at least very similar (e.g., for ten possible object features, each class may have a probability value of 0.1). With the initial weights, the neural network 4610 is unable to determine low level features and thus cannot make an accurate determination of what the classification of the object might be. A loss function can be used to analyze errors in the output. Any suitable loss function definition can be used.

The loss (or error) can be high for the first training dataset (e.g., images) since the actual values will be different than the predicted output. The goal of training is to minimize the amount of loss so that the predicted output comports with a target or ideal output. The neural network 4610 can perform a backward pass by determining which inputs (weights) most contributed to the loss of the neural network 4610 and can adjust the weights so that the loss decreases and is eventually minimized.

A derivative of the loss with respect to the weights can be computed to determine the weights that contributed most to the loss of the neural network 4610. After the derivative is computed, a weight update can be performed by updating the weights of the filters. For example, the weights can be updated so that they change in the opposite direction of the gradient. A learning rate can be set to any suitable value, with a high learning rate including larger weight updates and a lower value indicating smaller weight updates.

The neural network 4610 can include any suitable neural or deep learning network. One example includes a convolutional neural network (CNN), which includes an input layer and an output layer, with multiple hidden layers between the input and out layers. The hidden layers of a CNN include a series of convolutional, nonlinear, pooling (for downsampling), and fully connected layers. In other examples, the neural network 4610 can represent any other neural or deep learning network, such as an autoencoder, a deep belief nets (DBNs), a recurrent neural networks (RNNs), etc.

In some embodiments, an initial dataset will be built using the neural network architecture 4600, and further data from system usage is fed back into the corresponding models by using practical secure aggregation. As such, the models can be encrypted in such a way that even the (cloud) server responsible for aggregating the models will be unable to decrypt the learnings of each model. The practical secure aggregation can be combined with differential privacy and federated learning to minimize any chance of leaking protected health information while still being able to utilize the advancements of modern machine learning.

In one example, a data lake is used to receive data from an application of a network device. Sensitive data will be anonymized, reprocessed, or aggregated before being sent to the data lake. All the data can be stored on the data lake as is. Accordingly, the system can return to original data after applying a data transformation. In another example, a data versioning system is used. The data versioning system (e.g., Pachyderm) stores and versions data in a format that is directly consumable by the ML tools and pipelines. The data versioning system can also version the artifacts (models and code) that are produced by the pipelines and that should be sent in an update procedure. Note that the data acquired will be encrypted while in transit and will be stored fully disconnected from the network devices (e.g., the smartglasses 106, the application computing system 122, the computing device 125, the healthcare facility computing system 124, and the remote client computing device 118) to ensure the data is properly isolated (ensuring HIPAA compliance). This secure data aggregation, communication, and versioning using the neural network architecture 4600 may be carried out by the secure data/communication module 314 of the device management application 216.

Although deep learning and convolutional neural networks (CNN) are described as two technologies commonly used to accomplish image recognition for computer vision applications, it should be appreciated that any other suitable AI platforms and AI models now known or later developed may be used to accomplish such image recognition or data processing.

### Computing Device Hardware Architecture

Generally, the techniques disclosed herein may be implemented on hardware or a combination of software and hardware. For example, they may be implemented in an operating system kernel, in a separate user process, in a library package bound into network applications, on a specially constructed machine, on an application-specific integrated circuit (ASIC), or on a network interface card.

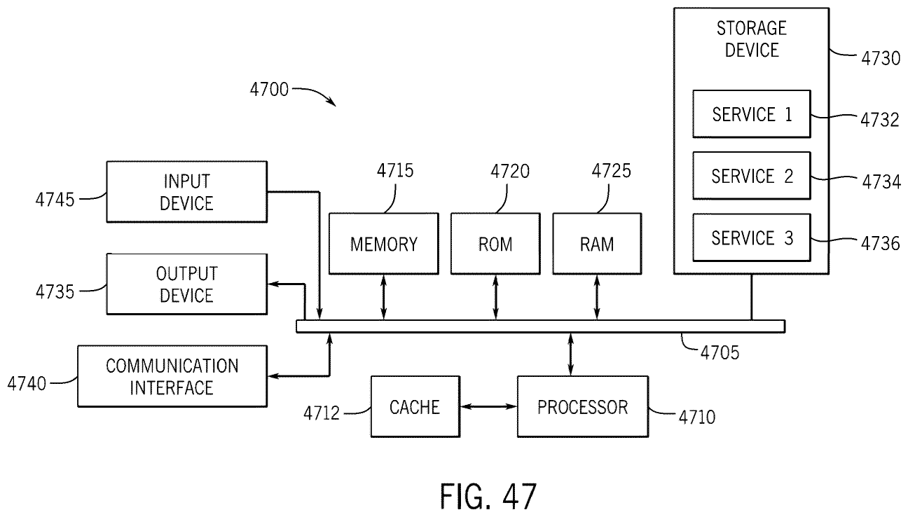

FIG. 47 is a diagram illustrating an example of a system for implementing certain aspects of the present technology. In particular, FIG. 47 illustrates an example of computing system 4700, which can be for example any computing device making up internal computing system, a remote computing system, or any component thereof in which the components of the system are in communication with each other using connection 4705. Connection 4705 can be a physical connection using a bus, or a direct connection into processor 4710, such as in a chipset architecture. Connection 4705 can also be a virtual connection, networked connection, or logical connection.

In some embodiments, computing system 4700 is a distributed system in which the functions described in this disclosure can be distributed within a datacenter, multiple data centers, a peer network, etc. In some embodiments, one or more of the described system components represents many such components each performing some or all of the function for which the component is described. In some embodiments, the components can be physical or virtual devices.

Example system 4700 includes at least one processing unit (CPU or processor) 4710 and connection 4705 that couples various system components including system memory 4715, such as read-only memory (ROM) 4720 and random access memory (RAM) 4725 to processor 4710. Computing system 4700 can include a cache 4712 of high-speed memory connected directly with, in close proximity to, or integrated as part of processor 4710.

Processor 4710 can include any general purpose processor and a hardware service or software service, such as services 4732, 4734, and 4736 stored in storage device 4730, configured to control processor 4710 as well as a special-purpose processor where software instructions are incorporated into the actual processor design. Processor 4710 may essentially be a completely self-contained computing system, containing multiple cores or processors, a bus, memory controller, cache, etc. A multi-core processor may be symmetric or asymmetric.

To enable user interaction, computing system 4700 includes an input device 4745, which can represent any number of input mechanisms, such as a microphone for speech, a touch-sensitive screen for gesture or graphical input, keyboard, mouse, motion input, speech, etc. Computing system 4700 can also include output device 4735, which can be one or more of a number of output mechanisms. In some instances, multimodal systems can enable a user to provide multiple types of input/output to communicate with computing system 4700.

Computing system 4700 can include communications interface 4740, which can generally govern and manage the user input and system output. The communication interface 4740 may perform or facilitate receipt and/or transmission wired or wireless communications using wired and/or wireless transceivers, including those making use of an audio jack/plug, a microphone jack/plug, a universal serial bus (USB) port/plug, an Apple® Lightning® port/plug, an Ethernet port/plug, a fiber optic port/plug, a proprietary wired port/plug, a BLUETOOTH® wireless signal transfer, a BLUETOOTH® low energy (BLE) wireless signal transfer, an IBEACON® wireless signal transfer, a radio-frequency identification (RFID) wireless signal transfer, near-field communications (NFC) wireless signal transfer, dedicated short range communication (DSRC) wireless signal transfer, 802.11 Wi-Fi wireless signal transfer, wireless local area network (WLAN) signal transfer, Visible Light Communication (VLC), Worldwide Interoperability for Microwave Access (WiMAX), Infrared (IR) communication wireless signal transfer, Public Switched Telephone Network (PSTN) signal transfer, Integrated Services Digital Network (ISDN) signal transfer, 3G/4G/5G/LTE cellular data network wireless signal transfer, ad-hoc network signal transfer, radio wave signal transfer, microwave signal transfer, infrared signal transfer, visible light signal transfer, ultraviolet light signal transfer, wireless signal transfer along the electromagnetic spectrum, or some combination thereof.

In some implementations, the communication interface 4740 can provide for communications under various modes or protocols, such as Global System for Mobile communication (GSM) voice calls, Short Message Service (SMS), Enhanced Messaging Service (EMS), or Multimedia Messaging Service (MMS) messaging, Code Division Multiple Access (CDMA), Time Division Multiple Access (TDMA), Personal Digital Cellular (PDC), Wideband Code Division Multiple Access (WCDMA), CDMA2000, or General Packet Radio System (GPRS), among others. For example, the communication may occur through a radio-frequency transceiver (not shown).

The communications interface 4740 may also include one or more Global Navigation Satellite System (GNSS) receivers or transceivers that are used to determine a location of the computing system 4700 based on receipt of one or more signals from one or more satellites associated with one or more GNSS systems. GNSS systems include, but are not limited to, the US-based Global Positioning System (GPS), the Russia-based Global Navigation Satellite System (GLONASS), the China-based BeiDou Navigation Satellite System (BDS), and the Europe-based Galileo GNSS. There is no restriction on operating on any particular hardware arrangement, and therefore the basic features here may easily be substituted for improved hardware or firmware arrangements as they are developed.

Storage device 4730 can be a non-volatile and/or non-transitory and/or computer-readable memory device and can be a hard disk or other types of computer readable media which can store data that are accessible by a computer, such as magnetic cassettes, flash memory cards, solid state memory devices, digital versatile disks, cartridges, a floppy disk, a flexible disk, a hard disk, magnetic tape, a magnetic strip/stripe, any other magnetic storage medium, flash memory, memristor memory, any other solid-state memory, a compact disc read only memory (CD-ROM) optical disc, a rewritable compact disc (CD) optical disc, digital video disk (DVD) optical disc, a blu-ray disc (BDD) optical disc, a holographic optical disk, another optical medium, a secure digital (SD) card, a micro secure digital (microSD) card, a Memory Stick® card, a smartcard chip, a EMV chip, a subscriber identity module (SIM) card, a mini/micro/nano/pico SIM card, another integrated circuit (IC) chip/card, random access memory (RAM), static RAM (SRAM), dynamic RAM (DRAM), read-only memory (ROM), programmable read-only memory (PROM), erasable programmable read-only memory (EPROM), electrically erasable programmable read-only memory (EEPROM), flash EPROM (FLASHEPROM), cache memory (L1/L2/L3/L4/L5/L #), resistive random-access memory (RRAM/ReRAM), phase change memory (PCM), spin transfer torque RAM (STT-RAM), another memory chip or cartridge, and/or a combination thereof.

The storage device 4730 can include software services, servers, services, etc., that when the code that defines such software is executed by the processor 4710, it causes the system to perform a function. In some embodiments, a hardware service that performs a particular function can include the software component stored in a computer-readable medium in connection with the necessary hardware components, such as processor 4710, connection 4705, output device 4735, etc., to carry out the function.

As used herein, the term “computer-readable medium” includes, but is not limited to, portable or non-portable storage devices, optical storage devices, and various other mediums capable of storing, containing, or carrying instruction(s) and/or data. A computer-readable medium may include a non-transitory medium in which data can be stored and that does not include carrier waves and/or transitory electronic signals propagating wirelessly or over wired connections. Examples of a non-transitory medium may include, but are not limited to, a magnetic disk or tape, optical storage media such as compact disk (CD) or digital versatile disk (DVD), flash memory, memory or memory devices. A computer-readable medium may have stored thereon code and/or machine-executable instructions that may represent a procedure, a function, a subprogram, a program, a routine, a subroutine, a module, a software package, a class, or any combination of instructions, data structures, or program statements. A code segment may be coupled to another code segment or a hardware circuit by passing and/or receiving information, data, arguments, parameters, or memory contents. Information, arguments, parameters, data, etc. may be passed, forwarded, or transmitted using any suitable means including memory sharing, message passing, token passing, network transmission, or the like.

In some embodiments the computer-readable storage devices, mediums, and memories can include a cable or wireless signal containing a bit stream and the like. However, when mentioned, non-transitory computer-readable storage media expressly exclude media such as energy, carrier signals, electromagnetic waves, and signals per se.

Specific details are provided in the description above to provide a thorough understanding of the embodiments and examples provided herein. However, it will be understood by one of ordinary skill in the art that the embodiments may be practiced without these specific details. For clarity of explanation, in some instances the present technology may be presented as including individual functional blocks including functional blocks comprising devices, device components, steps or routines in a method embodied in software, or combinations of hardware and software. Additional components may be used other than those shown in the FIGS. and/or described herein. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

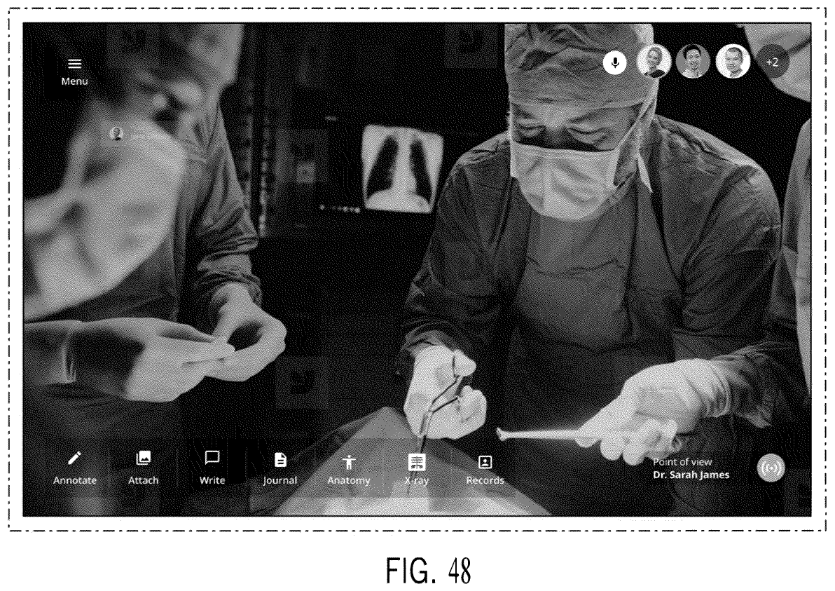

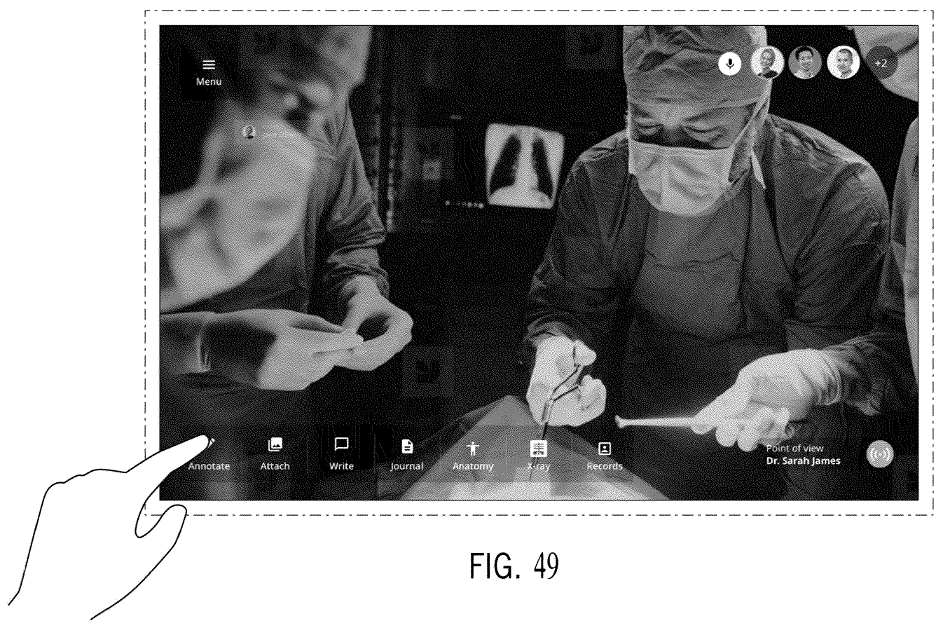

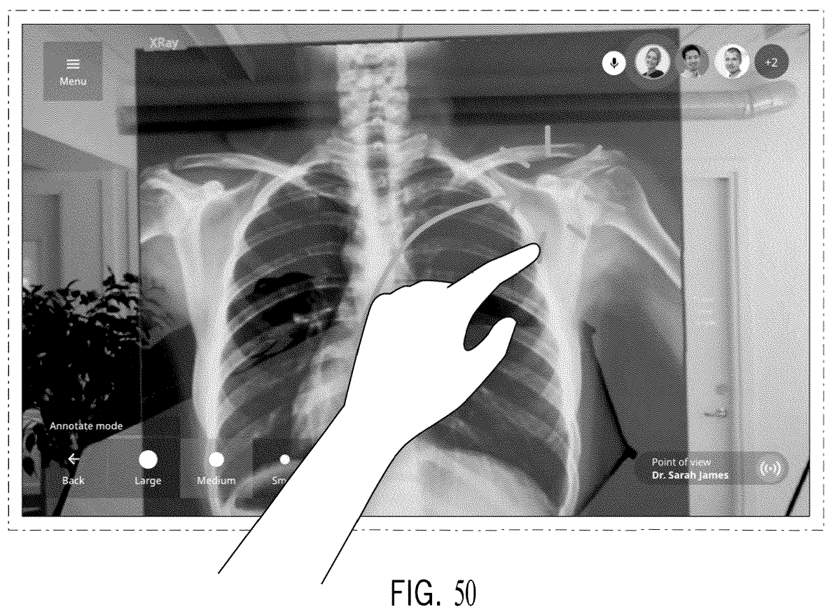

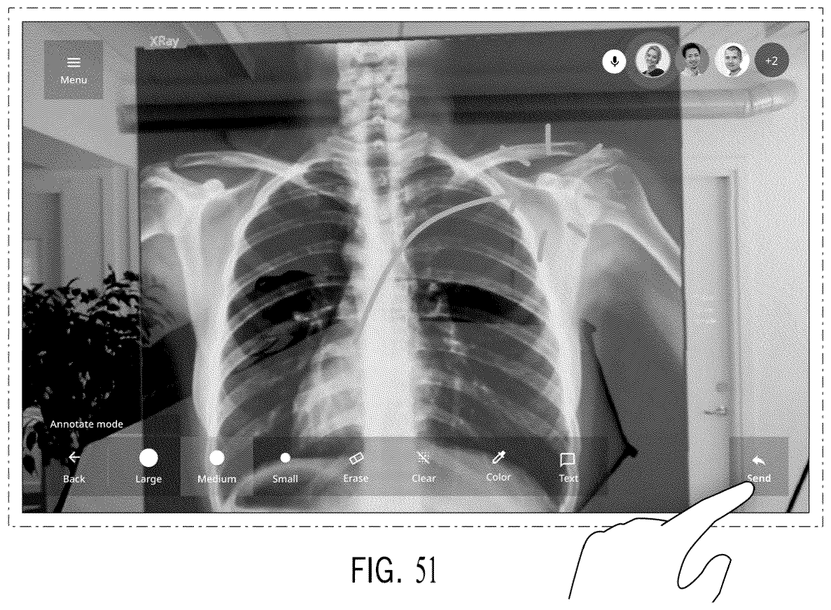

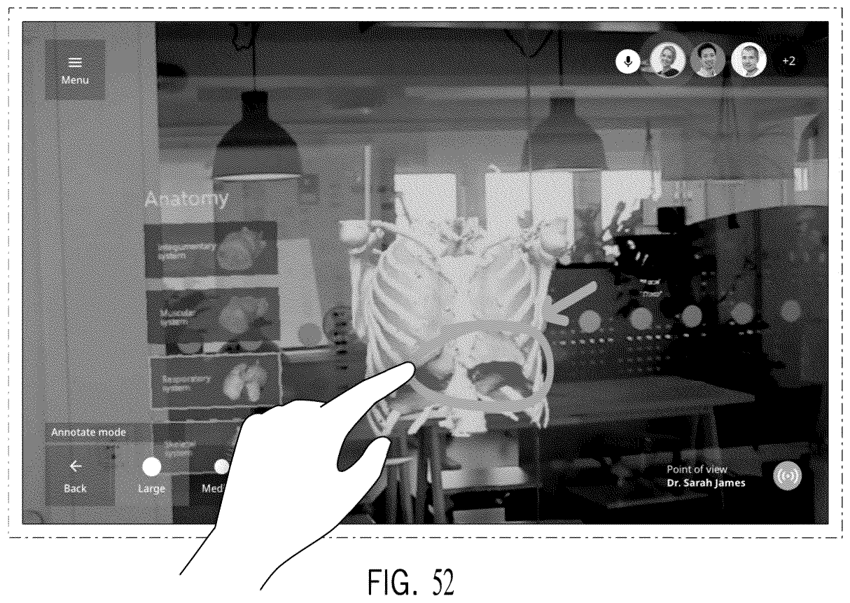

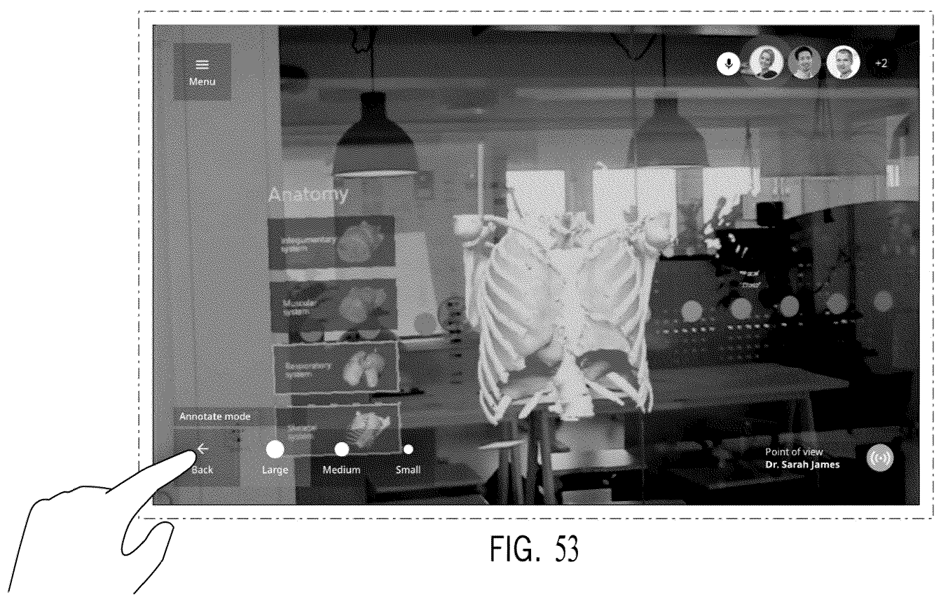

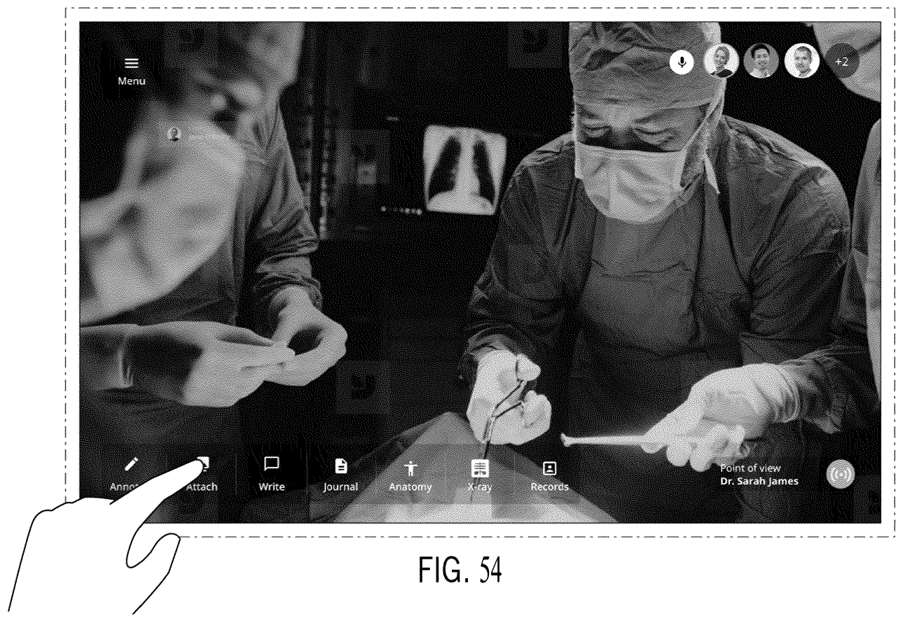

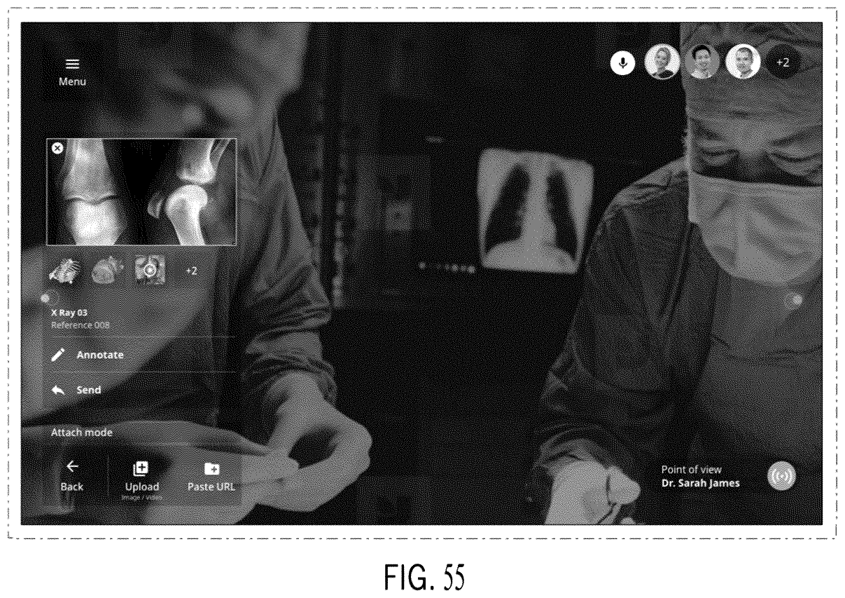

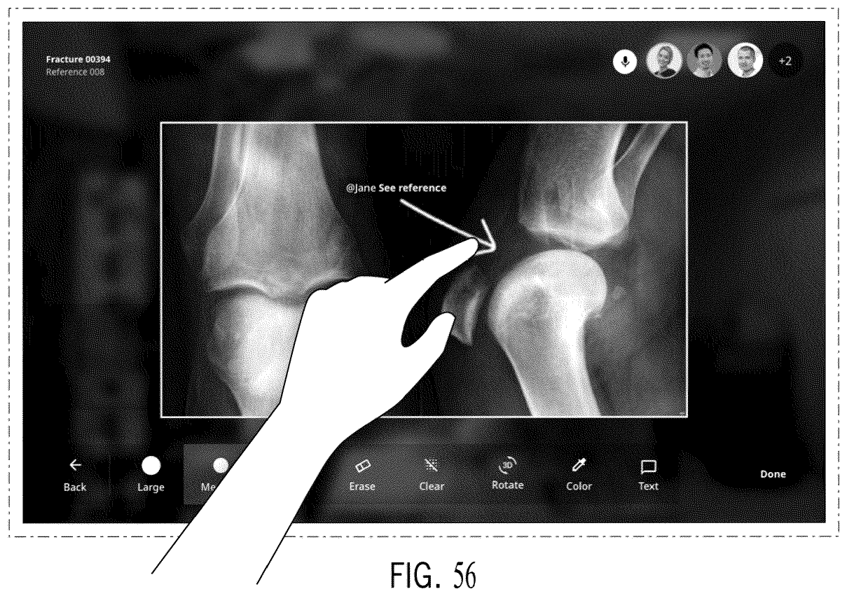

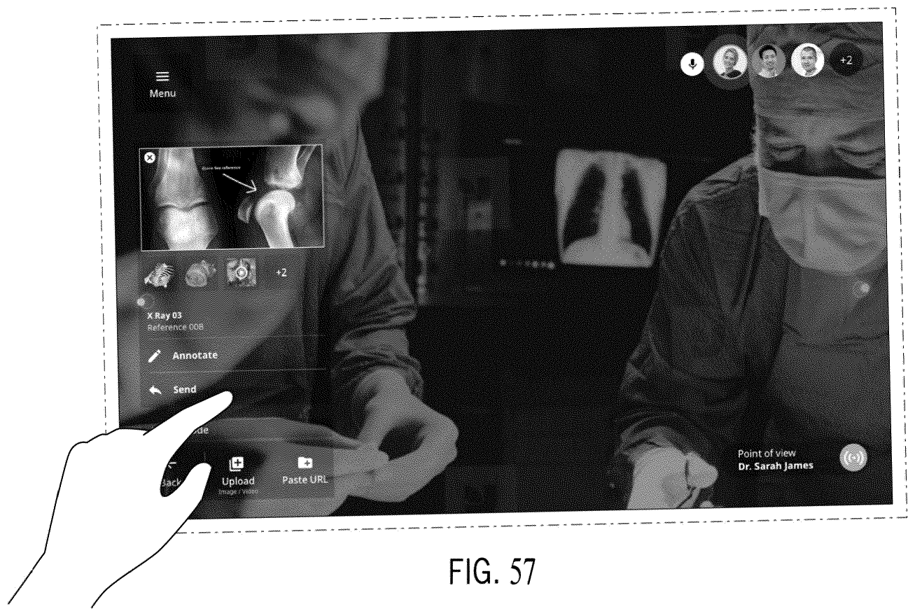

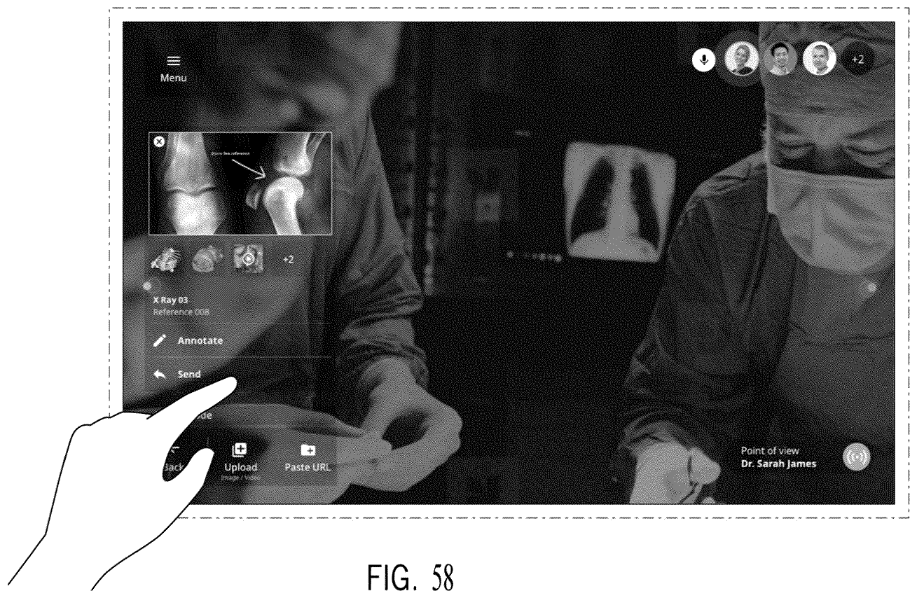

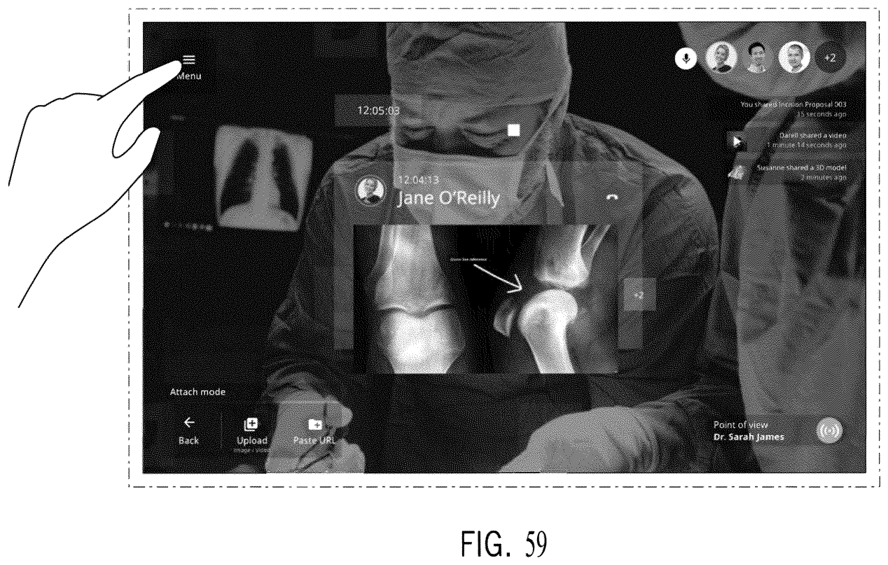

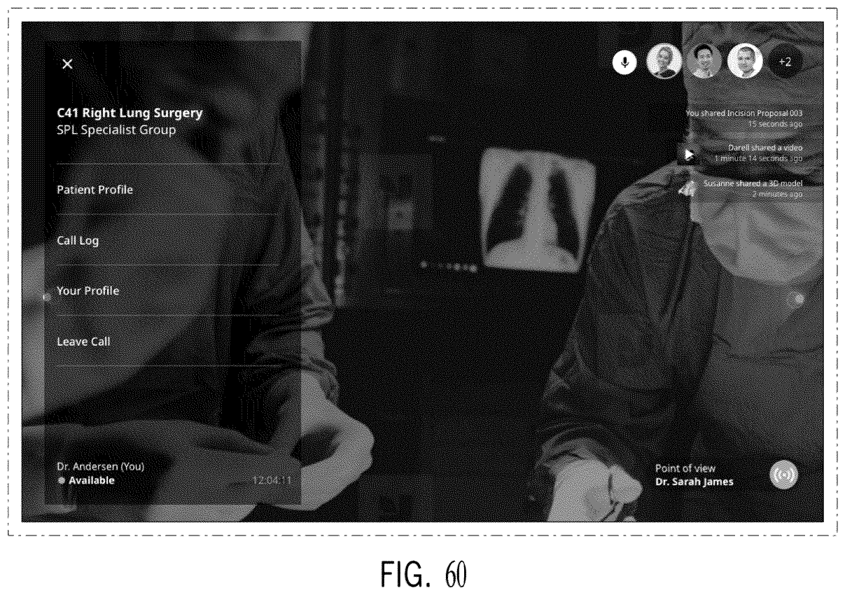

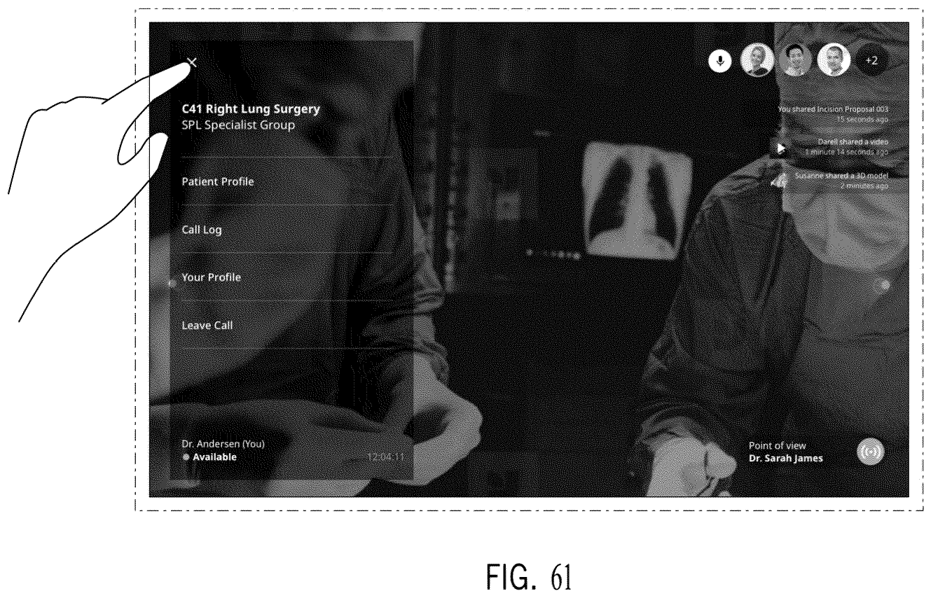

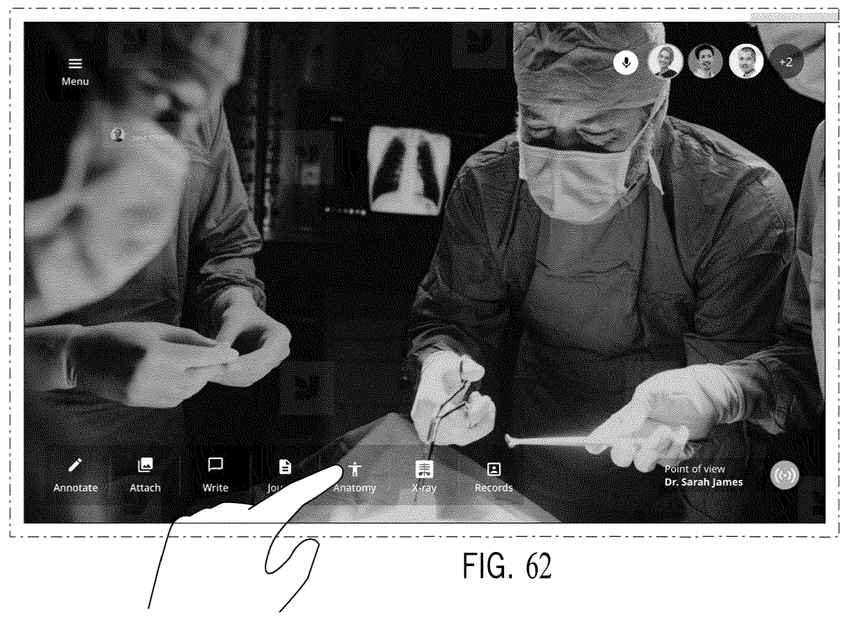

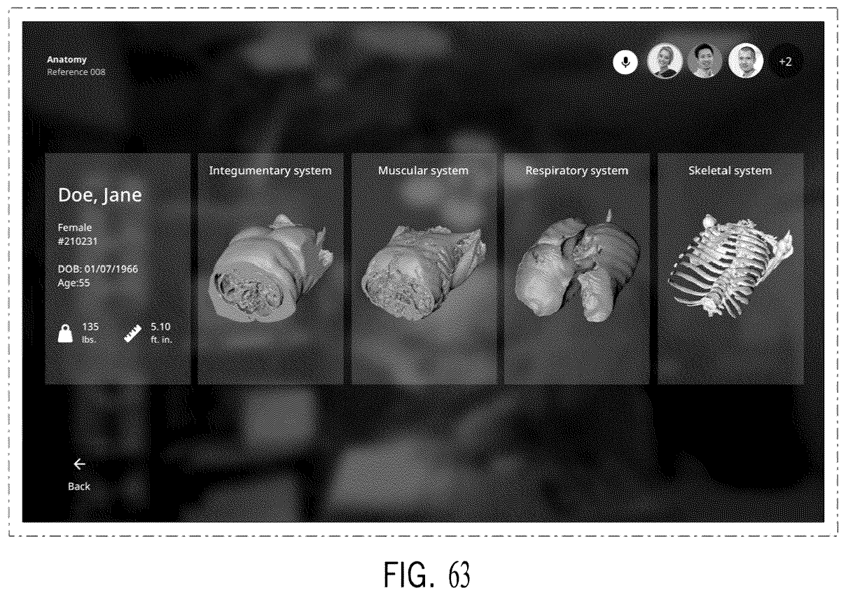

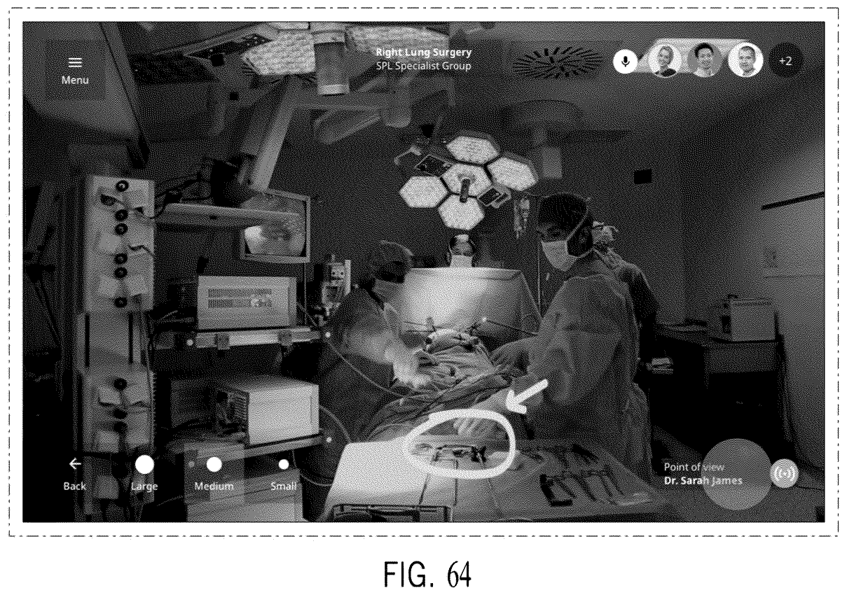

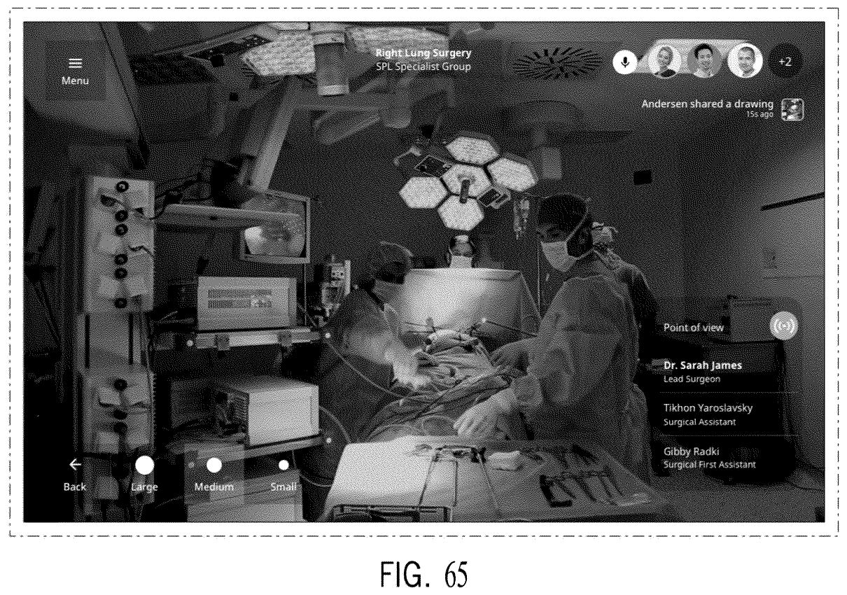

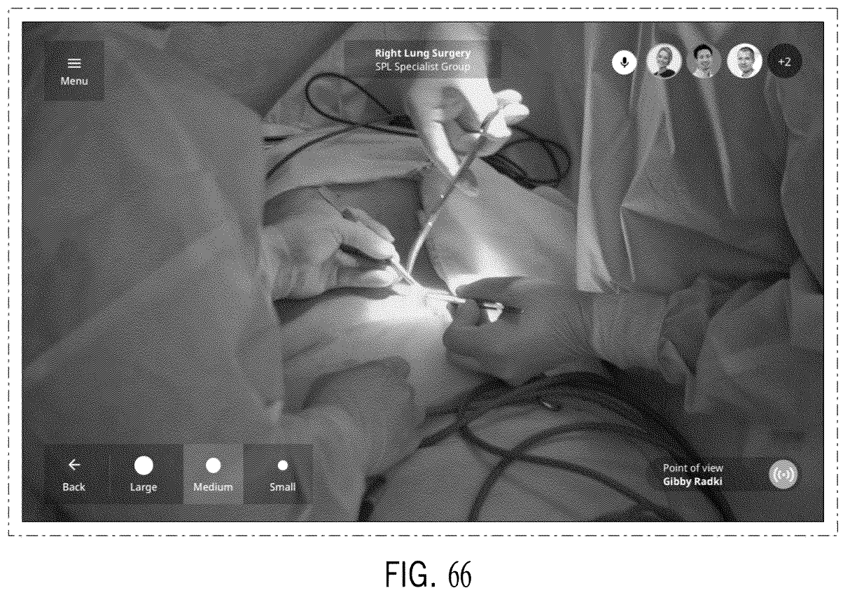

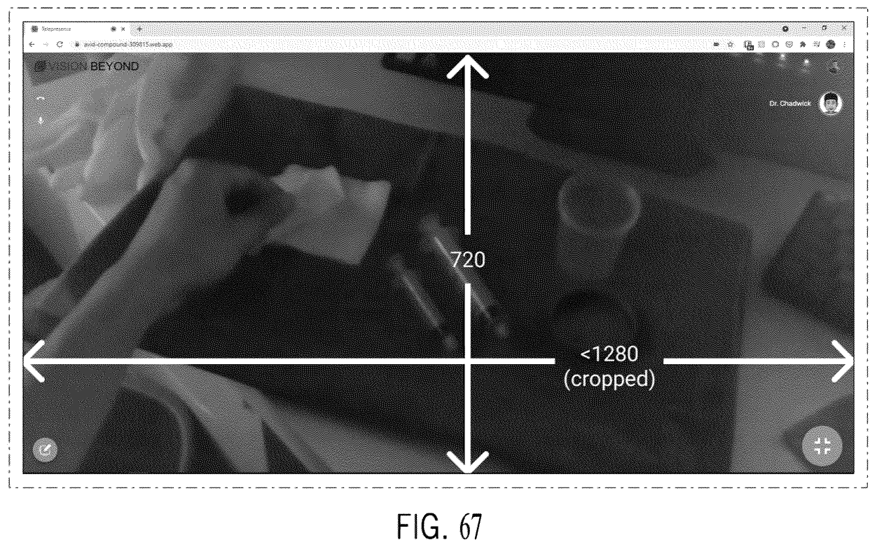

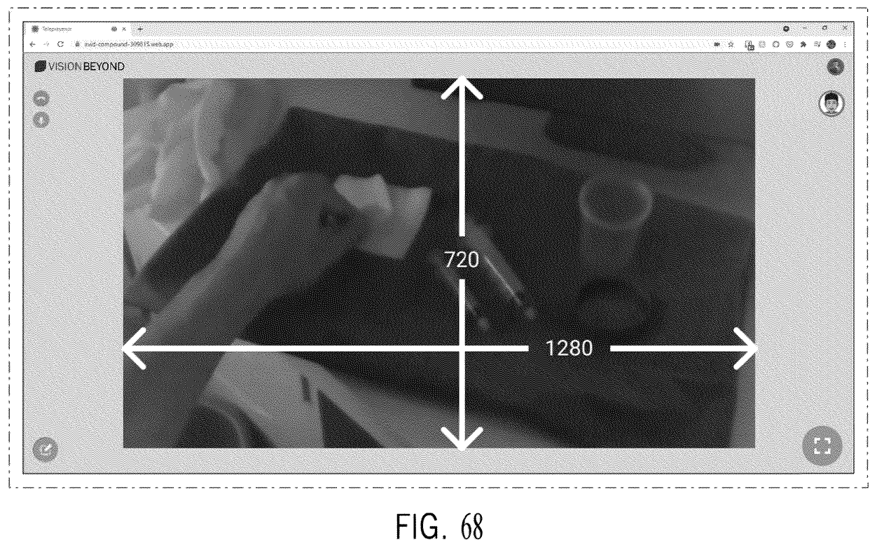

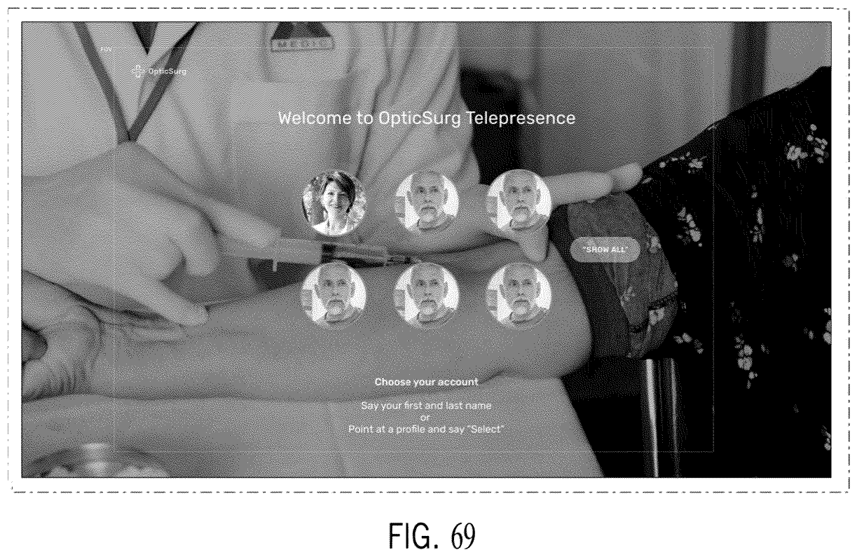

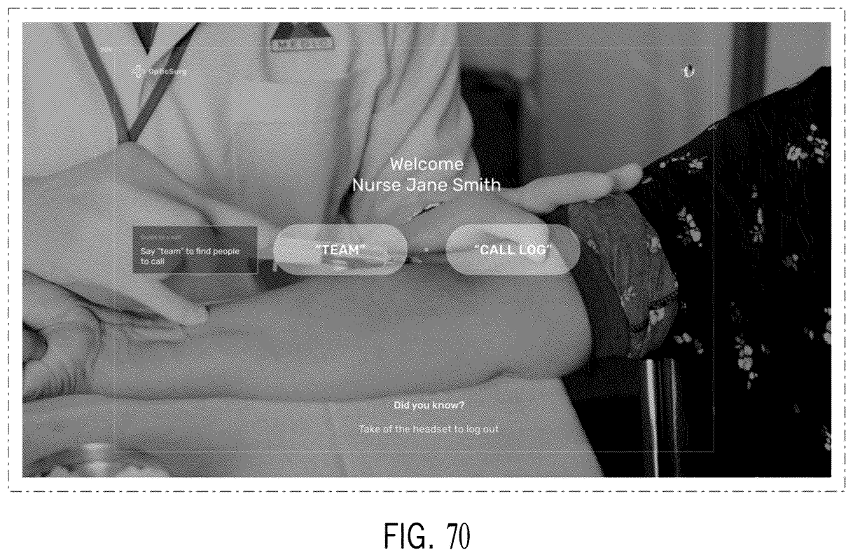

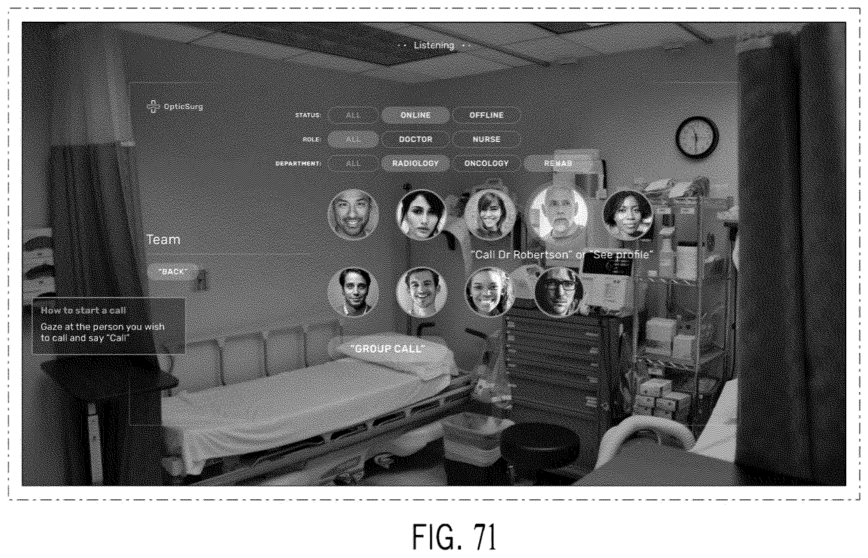

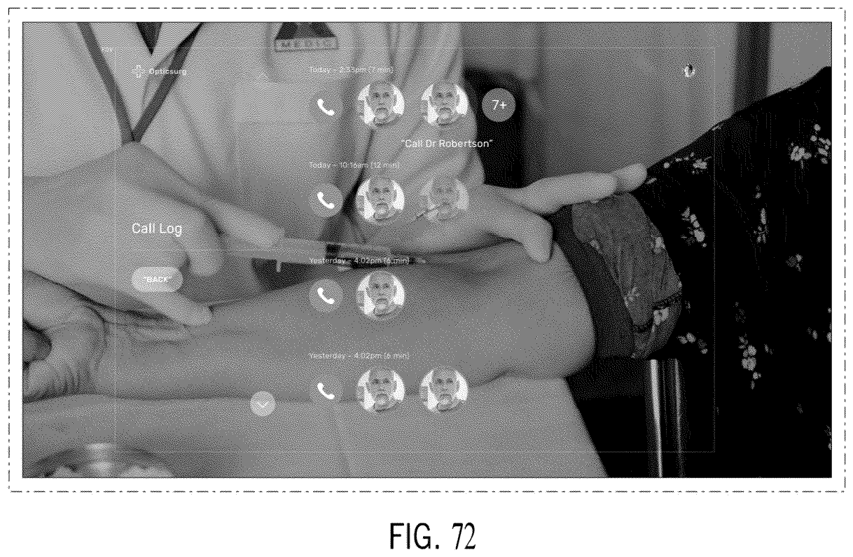

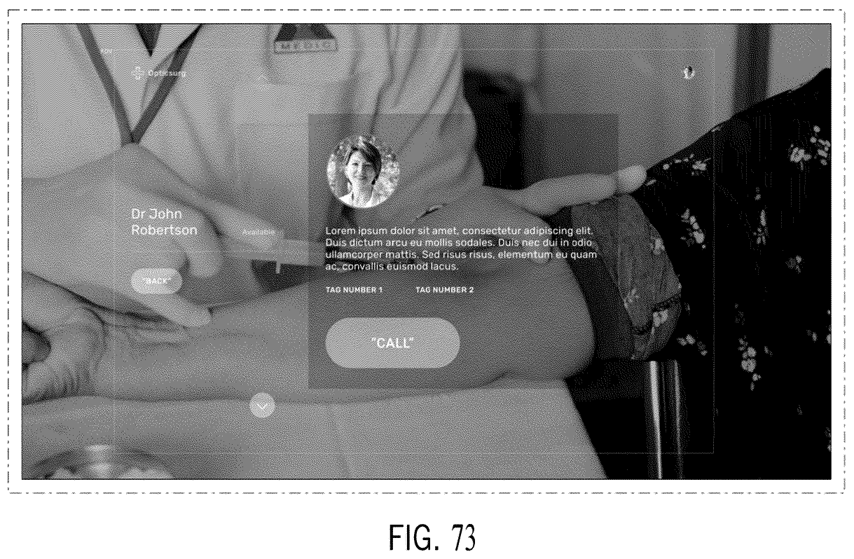

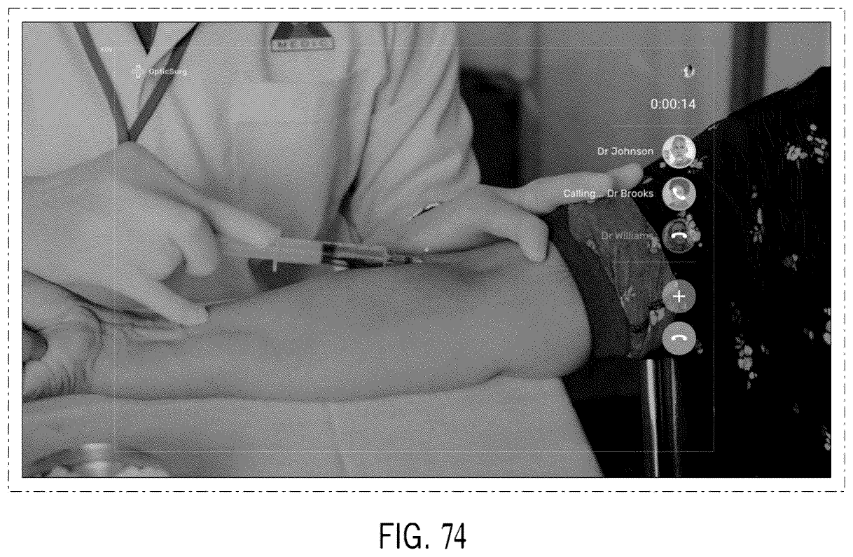

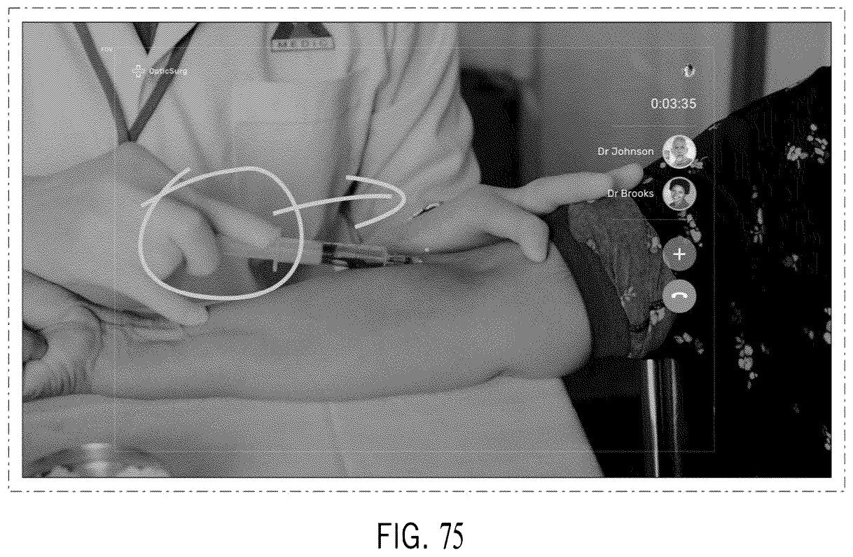

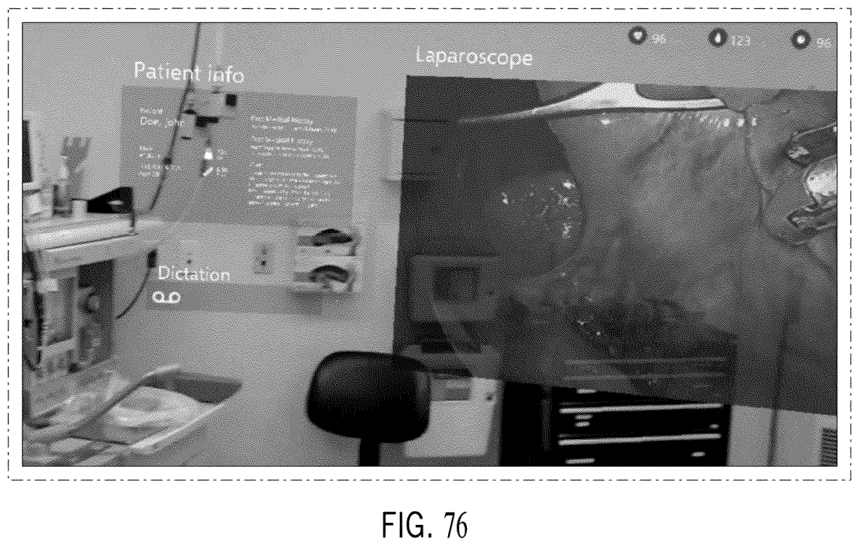

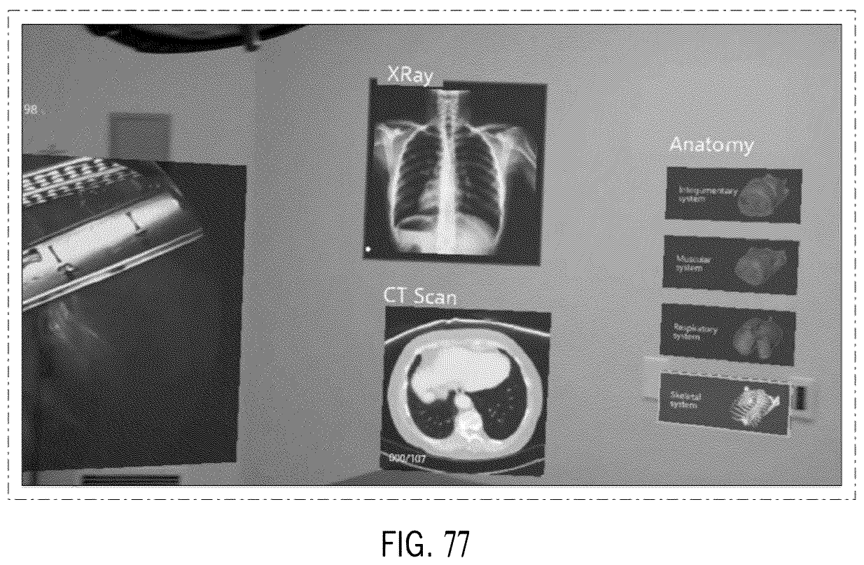

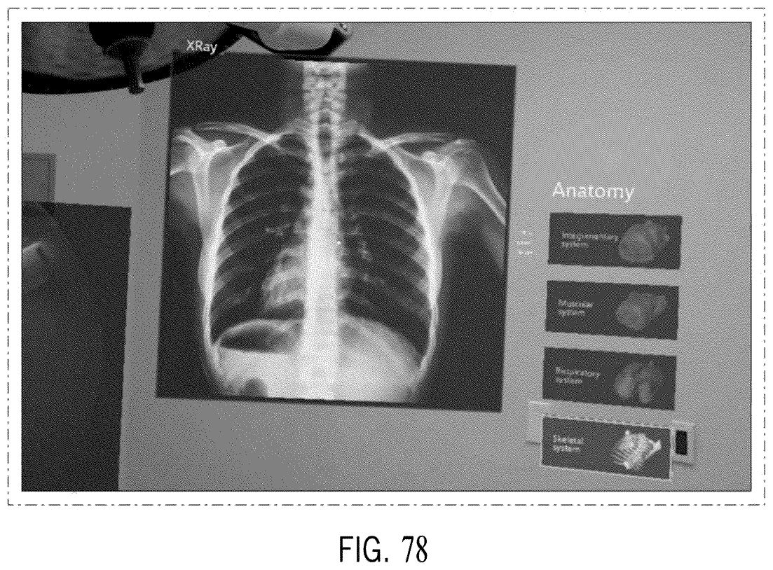

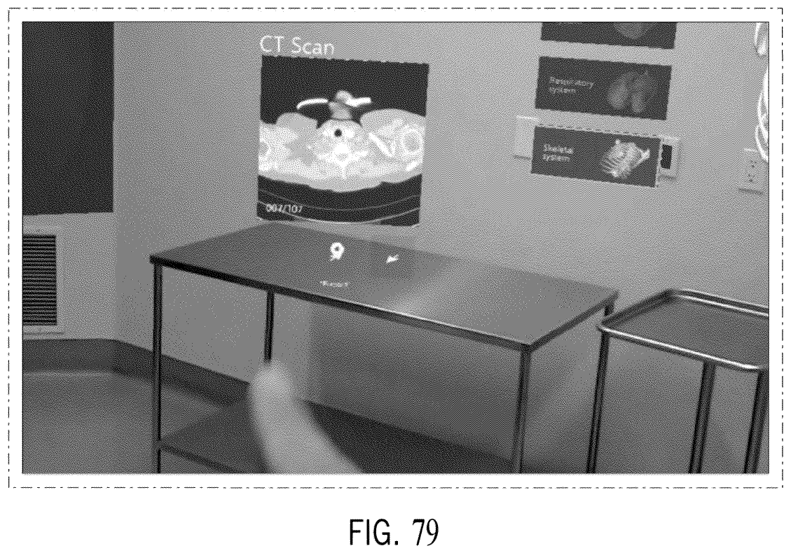

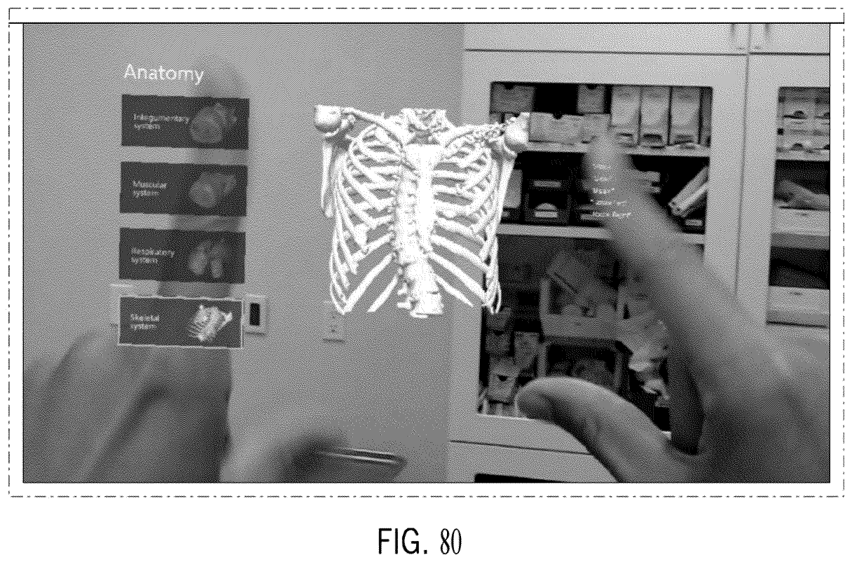

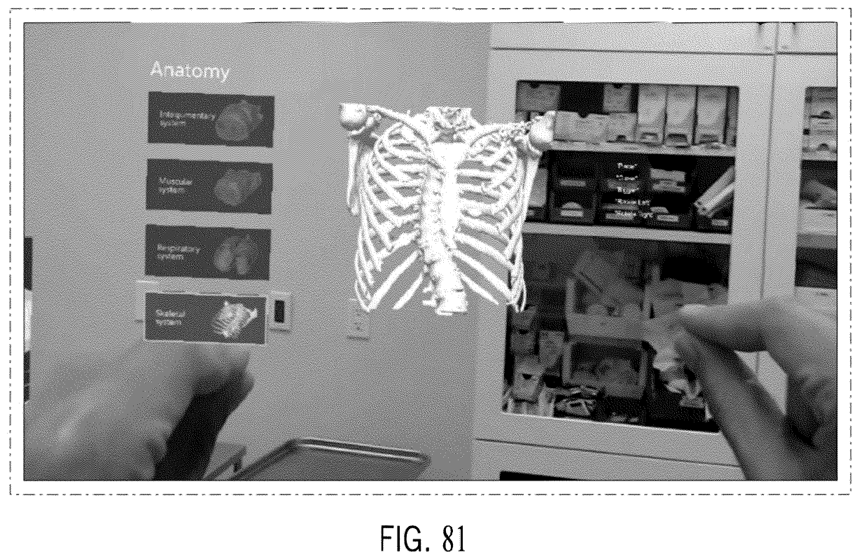

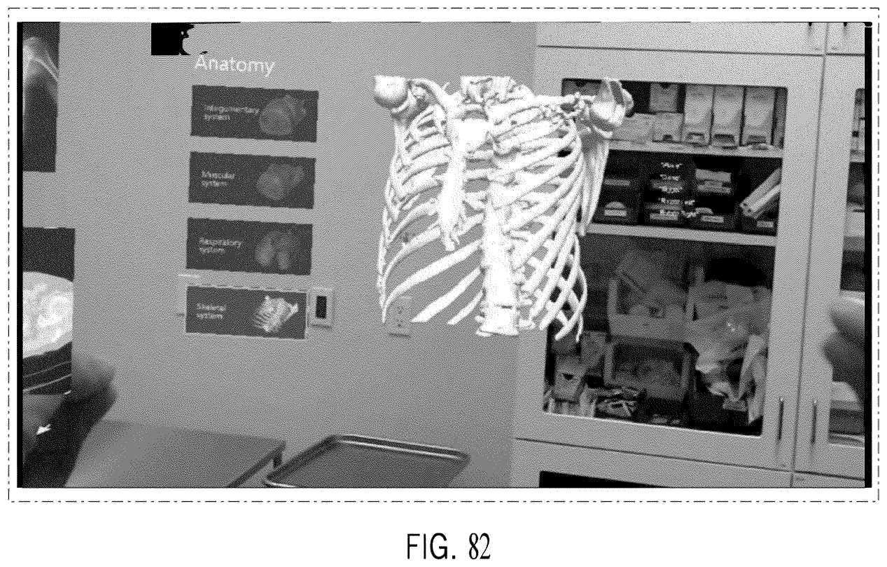

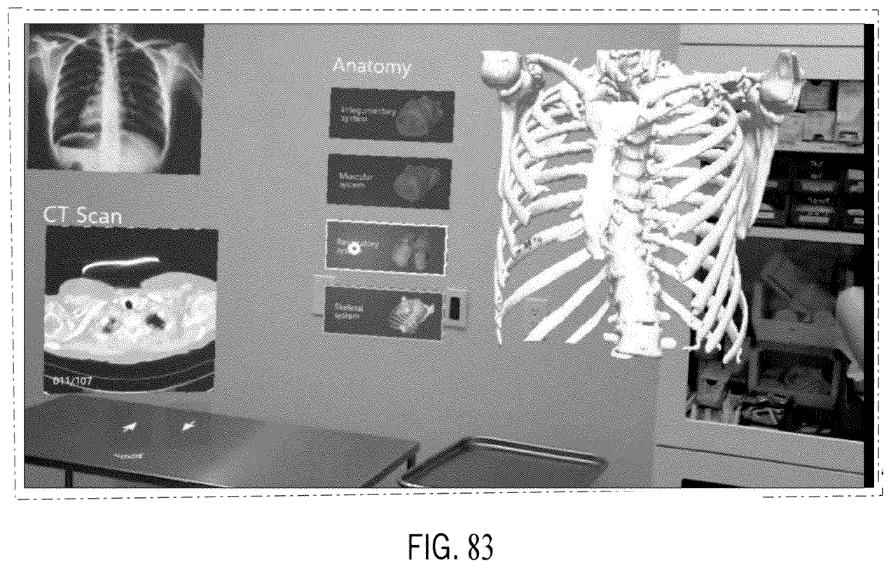

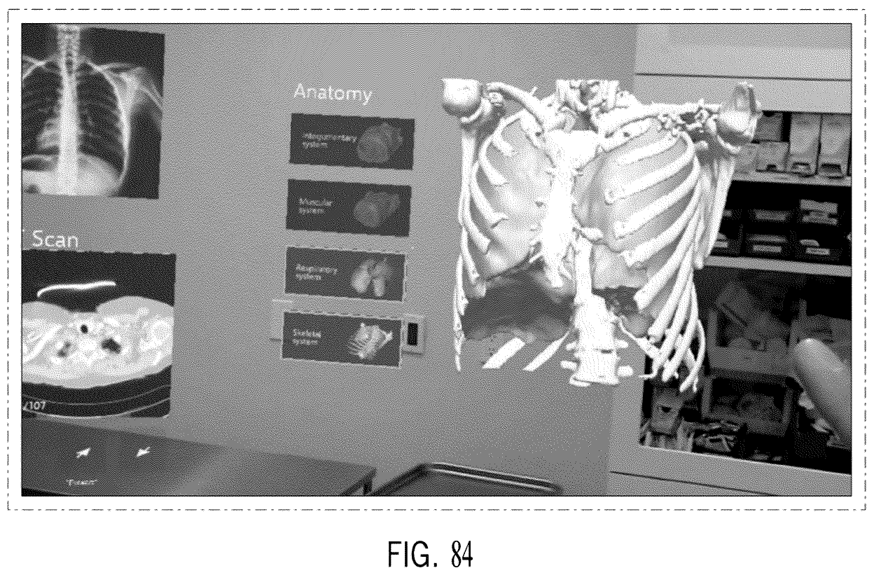

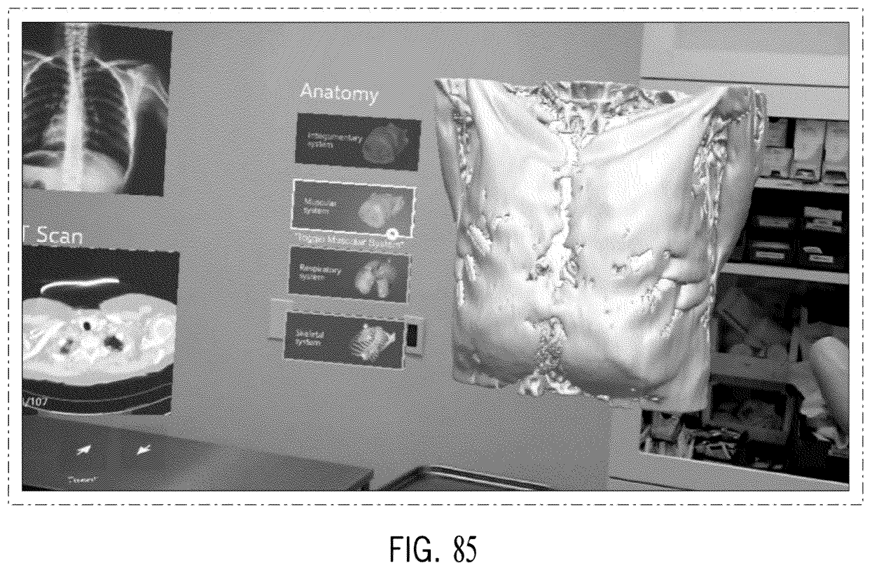

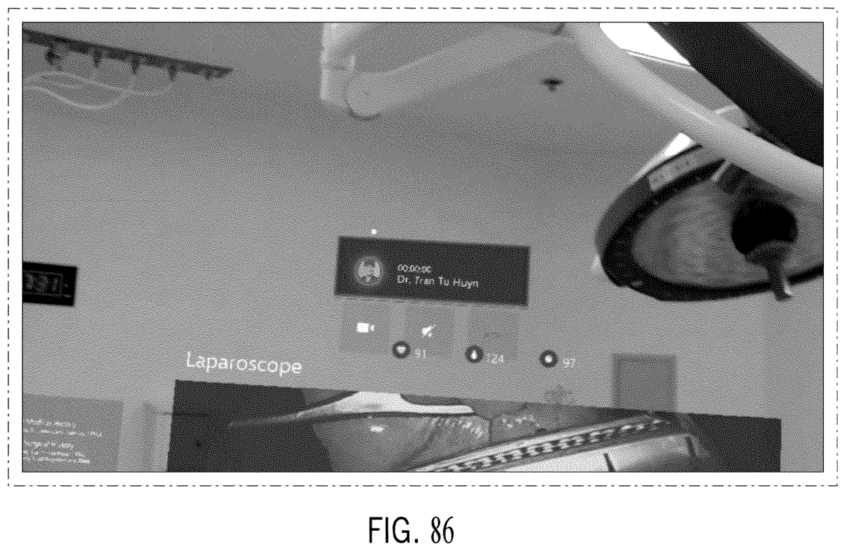

Individual embodiments may be described above as a process or method which is depicted as a flowchart, a swim diagram, a data flow diagram, a structure diagram, or a block diagram. Although a flowchart may describe the operations as a sequential process, many of the operations can be performed in parallel or concurrently. In addition, the order of the operations may be re-arranged. A process is terminated when its operations are completed, but could have additional steps not included in a FIG. 48 process may correspond to a method, a function, a procedure, a subroutine, a subprogram, etc. When a process corresponds to a function, its termination can correspond to a return of the function to the calling function or the main function.

Processes and methods according to the above-described examples can be implemented using computer-executable instructions that are stored or otherwise available from computer-readable media. Such instructions can include, for example, instructions and data which cause or otherwise configure a general purpose computer, special purpose computer, or a processing device to perform a certain function or group of functions. Portions of computer resources used can be accessible over a network. The computer executable instructions may be, for example, binaries, intermediate format instructions such as assembly language, firmware, source code, etc. Examples of computer-readable media that may be used to store instructions, information used, and/or information created during methods according to described examples include magnetic or optical disks, flash memory, USB devices provided with non-volatile memory, networked storage devices, and so on.

Devices implementing processes and methods according to these disclosures can include hardware, software, firmware, middleware, microcode, hardware description languages, or any combination thereof, and can take any of a variety of form factors. When implemented in software, firmware, middleware, or microcode, the program code or code segments to perform the necessary tasks (e.g., a computer-program product) may be stored in a computer-readable or machine-readable medium. A processor(s) may perform the necessary tasks. Typical examples of form factors include laptops, smart phones, mobile phones, tablet devices or other small form factor personal computers, personal digital assistants, rackmount devices, standalone devices, and so on. Functionality described herein also can be embodied in peripherals or add-in cards. Such functionality can also be implemented on a circuit board among different chips or different processes executing in a single device, by way of further example.

The instructions, media for conveying such instructions, computing resources for executing them, and other structures for supporting such computing resources are example means for providing the functions described in the disclosure.

The various illustrative logical blocks, modules, circuits, and algorithm steps described in connection with the embodiments disclosed herein may be implemented as electronic hardware, computer software, firmware, or combinations thereof. To clearly illustrate this interchangeability of hardware and software, various illustrative components, blocks, modules, circuits, and steps have been described above generally in terms of their functionality. Whether such functionality is implemented as hardware or software depends upon the particular application and design constraints imposed on the overall system. Skilled artisans may implement the described functionality in varying ways for each particular application, but such implementation decisions should not be interpreted as causing a departure from the scope of the present application.

The techniques described herein may also be implemented in electronic hardware, computer software, firmware, or any combination thereof. Such techniques may be implemented in any of a variety of devices such as general purposes computers, wireless communication device handsets, or integrated circuit devices having multiple uses including application in wireless communication device handsets and other devices. Any features described as modules or components may be implemented together in an integrated logic device or separately as discrete but interoperable logic devices. If implemented in software, the techniques may be realized at least in part by a computer-readable data storage medium comprising program code including instructions that, when executed, performs one or more of the methods described above. The computer-readable data storage medium may form part of a computer program product, which may include packaging materials. The computer-readable medium may comprise memory or data storage media, such as random access memory (RAM) such as synchronous dynamic random access memory (SDRAM), read-only memory (ROM), non-volatile random access memory (NVRAM), electrically erasable programmable read-only memory (EEPROM), FLASH memory, magnetic or optical data storage media, and the like. The techniques additionally, or alternatively, may be realized at least in part by a computer-readable communication medium that carries or communicates program code in the form of instructions or data structures and that can be accessed, read, and/or executed by a computer, such as propagated signals or waves.

The program code may be executed by a processor, which may include one or more processors, such as one or more digital signal processors (DSPs), general purpose microprocessors, an application specific integrated circuits (ASICs), field programmable logic arrays (FPGAs), or other equivalent integrated or discrete logic circuitry. Such a processor may be configured to perform any of the techniques described in this disclosure. A general purpose processor may be a microprocessor; but in the alternative, the processor may be any conventional processor, controller, microcontroller, or state machine. A processor may also be implemented as a combination of computing devices (e.g., a combination of a DSP and a microprocessor, a plurality of microprocessors, one or more microprocessors in conjunction with a DSP core, or any other such configuration). Accordingly, the term “processor,” as used herein may refer to any of the foregoing structure, any combination of the foregoing structure, or any other structure or apparatus suitable for implementation of the techniques described herein. In addition, in some aspects, the functionality described herein may be provided within dedicated software modules or hardware modules configured for encoding and decoding, or incorporated in a combined video encoder-decoder (CODEC).

While the concepts of the present disclosure are susceptible to various modifications and alternative forms, specific embodiments thereof have been shown by way of example in the drawings and will be described herein in detail. It should be understood, however, that there is no intent to limit the concepts of the present disclosure to the particular forms disclosed, but on the contrary, the intention is to cover all modifications, equivalents, and alternatives consistent with the present disclosure and the appended claims.

References in the specification to “one embodiment,” “an embodiment,” “an exemplary embodiment,” etc., indicate that the embodiment described may include a particular feature, structure, or characteristic, but every embodiment may or may not necessarily include that particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. Additionally, it should be appreciated that items included in a list in the form of “at least one A, B, and C” can mean (A); (B); (C); (A and B); (B and C); (A and C); or (A, B, and C). Similarly, items listed in the form of “at least one of A, B, or C” can mean (A); (B); (C); (A and B); (B and C); (A and C); or (A, B, and C).

Language such as “up”, “down”, “left”, “right”, etc., in the present disclosure is meant to provide orientation for the reader with reference to the drawings and is not intended to be the required orientation of the components or to impart orientation limitations into the claims.

In the drawings, some structural or method features may be shown in specific arrangements and/or orderings. However, it should be appreciated that such specific arrangements and/or orderings may not be required. Rather, in some embodiments, such features may be arranged in a different manner and/or order than shown in the illustrative FIGS. Additionally, the inclusion of a structural or method feature in a particular FIG. is not meant to imply that such feature is required in all embodiments and, in some embodiments, it may not be included or may be combined with other features.

As used herein, the terms “about” and “approximately,” in reference to a number, is used herein to include numbers that fall within a range of 10%, 5%, or 1% in either direction (greater than or less than) the number unless otherwise stated or otherwise evident from the context (except where such number would exceed 100% of a possible value).

Where electronic or software components are described as being “configured to” perform certain operations, such configuration can be accomplished, for example, by designing electronic circuits or other hardware to perform the operation, by programming programmable electronic circuits (e.g., microprocessors, or other suitable electronic circuits) to perform the operation, or any combination thereof.

The phrase “coupled to” refers to any component that is physically connected to another component either directly or indirectly, and/or any component that is in communication with another component (e.g., connected to the other component over a wired or wireless connection, and/or other suitable communication interface) either directly or indirectly.

Headings of sections provided in this patent application and the title of this patent application are for convenience only, and are not to be taken as limiting the disclosure in any way.

While preferred embodiments of the present invention have been shown and described herein, it will be apparent to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A communication management and information delivery system, comprising:
a wearable system configured to display virtual content in an AR environment, the wearable system comprising:
a sensor assembly having at least one sensor configured to capture medical multimedia data in connection with a medical session; and
a first display device configured to display virtual content in an AR environment; and
one or more processors configured to interact with non-transitory memory to perform operations comprising:
facilitating a secure communication between a remote client computing device and the wearable system, wherein the remote client computing device is configured to display captured medical multimedia data on a second display device;
generating feedback data associated with at least one of the captured medical multimedia data and electronic content displayed on the second display device of the remote client computing device, the feedback data generated based on feedback captured using one or more user input devices providing user input to the remote client computing device;
processing the feedback data with an annotation application including at least
an object type annotation module configured to automatically detect a current type of use or a current scene of a wearer of the wearable system and provide an annotation menu including a plurality of options for creating the annotation, the plurality of options forming the annotation menu being based on the automatically detected current use or scene of the wearer of the wearable system; and
displaying on the first display device of the wearable system the feedback data superimposed over a real-world view of at least one of the medical multimedia data, electronic content, and virtual content.

2. The system of claim 1, wherein an annotation of a live annotation module is presented as a virtual object in the first display device of the wearable system until at least one of the one or more user input devices ceases receiving input and a wearer of the wearable system provides a command to remove the annotation.

3. The system of claim 1, wherein an annotation of an live annotation module is presented as a virtual object in the first display device of the wearable system for a predetermined period after the one or more user input devices ceases receiving input.

4. The system of claim 1, wherein a type of object being annotated is determined by a computer vision platform configured to process inputs received from the sensor assembly.

5. (canceled)

6. The system of claim 1, wherein the current use or scene of a wearer of the wearable system is determined by at least one of a computer vision platform configured to process inputs received from the sensor assembly, an input selected by a wearer of the wearable system, and an input selected by a remote user of the remote client computing device.

7-15. (canceled)

16. The system of claim 1, wherein the one or more processors configured to interact with non-transitory memory are further configured to perform operations comprising:
processing one or more visual data signals captured by the sensor assembly to produce a zoomed-in or zoomed-out image data for display on at least one of the first display device of the wearable system, the second display device of the remote client computing device, and a third display device of a healthcare facility computing system.

17. (canceled)

18. (canceled)

19. The system of claim 1, wherein the one or more processors configured to interact with non-transitory memory are further configured to perform operations comprising processing one or more requests for access to an electronic resource to produce the electronic content with a resources application including at least one of:
an EHR module configured to receive and process a request for access to patient information and records, wherein the EHR module is configured to retrieve the patient information
or record and send data containing the requested patient information or record to at least one of the wearable system and the remote client computing device for display as an image;
an image module configured to receive and process requests for an electronic image related to a patient, wherein the image module is configured to receive a request for an electronic image of the patient and send data containing the requested electronic image to at least one of the wearable system and the remote client computing device for display as an image;
an anatomy module configured to receive and process requests for displaying an anatomy model, wherein the anatomy module is configured to receive a request for a type or area of an anatomy model to be displayed and send data containing the requested type or area of the anatomy model to at least one of the wearable system and the remote client computing device for display as an image;
a point of view module configured to receive and process requests for changing from a point of view of a first wearer of the wearable system to a point of view of a second first wearer of the wearable system; and
an activity log module configured to display an electronic activity log of at least one the wearable system and the remote client computing device during a communication session.

20. (canceled)

21. The system of claim 19, wherein the annotation application is configured to add an annotation to the electronic content retrieved by the resources application.

22. The system of claim 1, wherein the one or more processors configured to interact with non-transitory memory are further configured to perform operations comprising processing one or more requests for measurement data with a measurement application including at least one of:
an AR output module configured to display a virtual measurement image on at least one of the first display device of the wearable system or the second display device of the remote client computing device;
an EHR output module configured to send measurement data to a healthcare facility computing system; and
a data analysis module configured to send measurement data to a computer vision platform trained to identify or analyze measured objects viewed by a wearer of the wearable system.

23. (canceled)

24. The system of claim 1, wherein the one or more processors configured to interact with non-transitory memory are further configured to perform operations comprising processing one or more requests to associate virtual objects with IoT devices with an IoT application including at least one of:
an IoT device library module configured to store information for the IoT devices associated with the wearable system;
an IoT AR module configured to represent output aspects of the IoT devices as a virtual object in the first display device of the wearable system; and
an IoT function module configured to control aspects of the IoT devices through inputs of a wearer of the wearable system.

25. The system of claim 1, wherein the one or more processors configured to interact with non-transitory memory are further configured to perform operations comprising processing one or more requests to receive, process, and package data for sending between at least the wearable system and the remote client computing device on a network with a data integration application including at least one of:
an EHR module configured to receive, process, and package data for sending between at least one of the wearable system and the remote client computing device and a healthcare facility computing system;
a medical device module configured to receive, process, and package data for sending between at least one of the wearable system and the remote client computing device and external medical devices; and
an external services module configured to receive, process, and package data for sending between at least one of the wearable system and the remote client computing device and a remote computer vision platform.

26. The system of claim 1, wherein the one or more processors configured to interact with non-transitory memory are further configured to perform operations comprising:
securing data sent between at least the wearable system and the remote client computing device on a network with a device management application including a secure data/communication module configured to exchange encrypted and anonymized information between the wearable system and the remote client computing device and any other device on the network.

27 -29. (canceled)

30. The system of claim 1, wherein the one or more processors configured to interact with non-transitory memory are further configured to perform operations comprising:
building one or more computer vision models; and
feeding data from usage of the communication management and information delivery system into corresponding models by using practical secure aggregation.

31. (canceled)

32. The system of claim 1, wherein the one or more processors configured to interact with non-transitory memory are further configured to perform operations comprising:
receiving data from an application of a network device with a data lake; and
using at least one of anonymization, reprocessing, or aggregation of data before the data is sent to the data lake.

33. (canceled)

34. The system of claim 1, wherein the one or more processors configured to interact with non-transitory memory are further configured to perform operations comprising:
receiving data from an application of a network device with a data versioning system; and
storing and versioning data in a format that is directly consumable by a computer vision platform.

35. (canceled)

36. The system of claim 1, wherein the one or more processors configured to interact with non-transitory memory are further configured to perform operations comprising:
detecting at least one input from a wearer of the wearable system; and
carrying out one or more commands for interacting with the virtual content based on the at least one input from the wearer.

37-39. (canceled)

40. The system of claim 1, wherein the remote client computing device includes a web browser application configured to facilitate interaction of the remote client computing device with the wearable system and other devices in network communication with the remote client computing device.

41-78. (canceled)

79. A method for facilitating a secure communication between a remote client computing device and a wearable system, wherein the wearable system is configured to display virtual content in an AR environment presented at a first display device forming a part of the wearable system, and capture medical multimedia data in connection with a medical session, and wherein the remote client computing device is configured to display captured medical multimedia data on a second display device, the method comprising:
generating feedback data associated with at least one of the captured medical multimedia data and electronic content displayed on the second display device of the remote client computing device, the feedback data generated based on feedback captured using one or more user input devices providing user input to the remote client computing device;
processing one or more requests to associate virtual objects with an Internet-of-Things (IoT) device by using an IoT application including at least one of:
an IoT device library configured to store information for the IoT device associated with the wearable system; or
an IoT function module configured to control aspects of the IoT device through inputs of a wearer of the wearable system;
processing the feedback data with an annotation application including at least one of:
a freeze frame module configured to add an annotation to a frozen video feed frame displayed on the second display device of the remote client computing device, the annotation corresponding to a 2-D location determined at the remote client computing device by the one or more user input devices providing user input to the remote client computing device;
a live annotation module configured to add an annotation to a live video feed frame displayed on the second display device of the remote client computing device, the annotation corresponding to a 2-D location determined at the remote client computing device by the one or more user input devices providing user input to the remote client computing device; or
a device specific annotation module configured to add an annotation corresponding to processing capabilities of the wearable system;
receiving the feedback data from the remote client computing device; and
displaying, on the first display device of the wearable system, the feedback data superimposed over a real-world view of at least one of the medical multimedia data, electronic content, and virtual content.

80-117. (canceled)

118. A method for facilitating communication between a remote client computing device and a wearable system, wherein the wearable system is configured to display virtual content in an AR environment presented at a first display device forming a part of the wearable system, and capture medical multimedia data in connection with a medical session, and wherein the remote client computing device is configured to display captured medical multimedia data on a second display device, the method comprising:
presenting, at the second display device of the remote client computing device, at least one of an annotation menu or an annotation toolset;
generating feedback data associated with at least one of the captured medical multimedia data and electronic content displayed on the second display device of the remote client computing device, the feedback data generated based on feedback captured using one or more user input devices providing user input to the annotation menu or the annotation toolset presented at the second display device of the remote client computing device;
using an Internet-of-Things (IoT) application to process one or more requests to associate virtual objects with an IoT device, the IoT application is configured to:
access an IoT device library storing information for the IoT device associated with the wearable system; and
control aspects of the IoT device through inputs of a wearer of the wearable system;
processing the feedback data with an annotation application including at least one of:
a freeze frame module configured to add an annotation to a frozen video feed frame displayed on the second display device of the remote client computing device, the annotation corresponding to a 2-D location of the one or more user input devices;
a live annotation module configured to add an annotation to a live video feed frame displayed on the second display device of the remote client computing device, the annotation corresponding to a 2-D location of the one or more user input devices; or
a device specific annotation module configured to add an annotation corresponding to processing capabilities of the wearable system;
receiving the feedback data from the remote client computing device; and
displaying the feedback data superimposed over a real-world view of at least one of the medical multimedia data, electronic content, or virtual content.
